SEQUENCE LISTING (119 sequences)

SEQ ID NO: 1 - DNA, 1218 bp, Arabidopsis thaliana
  G557 (HY5)
tcaaaggcttgcatcagcattagaaccaccaccacctcctctcttgtttcctgttgtgttcttcagaatctacaccacataaaaaacataacaactcaaaagactttattaccacacacacacatagagatccaactttgcaatctcatcttctccattcatatagaacaaaatgagtgagcatttcaagaaccattgaagaatttacatgccttttgagagaatatgcgagtgaatgaccatttcaagaacctacatgccttctgagaattaatctaaagcttaagttagcttcttagatccttttaactaactaaactaattattggtcaatcctagactcgtaaatgtgataaaccagtactgtgatatatcaaaaaacaaatggcaaaagcattgacgttgcaggttaagtcaacagtaagatcgacaaaacgtacatgtctaagcatctggttctcgttctgaagagtagagagtcgctcttcaagttcagagtttttgttctccaagtctttcactctgttttccaactcgctcaagtaagcctttttcctctctcttgcttgctgagctgaaactctgttcctcaacaaccttttcaccacaaaattaccaaacaaccccatcacgcaaccgttatttaacataatcaccttccatataaagggtaaaaatgtaaattcaatgaatagagaaaaagacacctcttcagccgcttgttctctttctccgccggtgtcctccctcgcttcctttgactttctccgacagtcgcctgtgtccgctcctgaccggtcgccgatccagattctctaccggaagtttcttttccgacagcttctcctccaaactccggcactcgccgtatctcctcatcgctttcaattcctttaaaacataaaagagactttagacgaaaagtttcaaactttttaaatacaataaaaaattgcagatcttctgggggagactaaaagttgtgaatctagatgtgaatcaatggtgatacaaaatctagatgtgaatttactagatatccaatgcatgagaatgaaaatcaatgagatcactcgttgggagaagatatgaaaataaaacaatcgacaatttttgtttaccttctttgatctccaaatgtggagcagagcttgatgacctctcgctgcttgatggtaaagagcttgcagctaaagagctagtcgcttgttcctgcat

SEQ ID NO: 2 - protein, 168 aa, Arabidopsis thaliana
  G557 (HY5) polypeptide
MQEQATSSLAASSLPSSSERSSSSAPHLEIKEGIESDEEIRRVPEFGGEAVGKETSGRESGSATGQERTQATVGESQRKRGRTPAEKENKRLKRLLRNRVSAQQARERKKAYLSELENRVKDLENKNSELEERLSTLQNENQMLRHILKNTTGNKRGGGGGSNADASL

SEQ ID NO: 3 - DNA, 604 bp, Arabidopsis thaliana
  G1809 (HYH)
ctctctattctcgtctttagcaaaatctcaaaagacaaaaagatattgatgtctctccaacgacccaatgggaactcgagttcgtcttcttcccacaagaagcacaaaactgaggaaagtgatgaggagttgttgatggttcctgacatggaagcagctggatcaacatgtgttctaagcagcagcgccgacgatggagtcaacaatccggagcttgaccagactcaaaatggagtctctacagctaaacgccgccgtggaagaaaccctgttgataaagaatatagaagcctcaagagattattgaggaacagagtatcagcgcaacaagcaagagagaggaagaaagtgtatgtgagtgatttggaatcaagagctaatgagttacagaacaacaatgaccagctcgaagagaagatttctactttgacgaacgagaacacaatgcttcgtaaaatgcttattaacacaaggcctaaaactgatgacaatcactaaatatttaccctttaatccattgttcagtgttgtatgattatctttctttcttttttggttttggtttgtatacactttttgttcgaataacattcactttgagcat

SEQ ID NO: 4 - protein, 149 aa, Arabidopsis thaliana
  G1809 (HYH) polypeptide
MSLQRPNGNSSSSSSHKKHKTEESDEELLMVPDMEAAGSTCVLSSSADDGVNNPELDQTQNGVSTAKRRRGRNPVDKEYRSLKRLLRNRVSAQQARERKKVYVSDLESRANELQNNNDQLEEKISTLTNENTMLRKMLINTRPKTDDNH

SEQ ID NO: 5 - DNA, 1262 bp, Glycine max
  G4631 (GmHY5-2; STF1b)
ggtttttgagaagaaagatggaacgaagtggcggaatggtaactgggtcgcatgaaaggaacgaacttgttagagttagacacggctctgatagtaggtctaaacccttgaagaatttgaatggtcagagttgtcaaatatgtggtgataccattggattaacggctactggtgatgtctttgtcgcttgtcatgagtgtggcttcccactttgtcattcttgttacgagtatgagctgaaacatatgagccagtcttgtccccagtgcaagactgcattcacaagtcaccaagagggtgctgaagtggaggagattgatatgatgaccgatgcttatctagataatgagatcaactatggccaaggaaacagttccaaggcggggatgctatgggaagaagatgctgacctctcttcatcttctggacatgattctcaaataccaaacccccatctagcaaacgggcaaccgatgtctggtgagtttccatgtgctacttctgatgctcaatctatgcaaactacatctataggtcaatccgaaaaggttcactcactttcatatgctgatccaaagcaaccaggtcctgagagtgatgaagagataagaagagtgccagagattggaggtgaaagtgccggaacttcggcctctcagccagatgccggttcaaatgctggtacagagcgtgttcaggggacaggggagggtcagaagaagagagggagaagcccagctgataaagaaagtaaacggctaaagaggctactgaggaaccgagtttcagctcagcaagcaagggagaggaagaaggcatacttgattgatttggaaacaagagtcaaagacttagagaagaagaactcagagctcaaagaaagactttccactttgcagaatgagaaccaaatgcttagacaaatattgaagaacacaacagcaagcaggagagggagcaataatggtaccaataatgctgagtgaacataatgtcaaaagatggcagagaaaacttatagatggaatagatttagaaagagagaatacattagccagaaagagaaaaaaaaattggacattagttgatgattctttctaggtgtgcgtttggaatacaatgaagtaaaggatgaaccttaagacatgctttatcctaaaatagtgtgatctgatattccattgttaatgagtaatgtaattatcatacaaacaatttgtagtctcattttaattaataattattaaactacttgattactt

SEQ ID NO: 6 - protein, 322 aa, Glycine max
  G4631 (GmHY5-2; STF1b) polypeptide
MERSGGMVTGSHERNELVRVRHGSDSRSKPLKNLNGQSCQICGDTIGLTATGDVFVACHECGFPLCHSCYEYELKHMSQSCPQCKTAFTSHQEGAEVEEIDMMTDAYLDNEINYGQGNSSKAGMLWEEDADLSSSSGHDSQIPNPHLANGQPMSGEFPCATSDAQSMQTTSIGQSEKVHSLSYADPKQPGPESDEEIRRVPEIGGESAGTSASQPDAGSNAGTERVQGTGEGQKKRGRSPADKESKRLKRLLRNRVSAQQARERKKAYLIDLETRVKDLEKKNSELKERLSTLQNENQMLRQILKNTTASRRGSNNGTNNAE

SEQ ID NO: 7 - DNA, 1317 bp, Oryza sativa
  G4627
ctagctcttggtgaaatggtgcttcttcccgccgccgccgccatcgccgcccttgcctccgccgccgccgcccctcttgccggcgtgcgccgtcgtgttcttgagtatctataggagagtagaggagaaatcgccatgagagattgagaatggtgaagcaaagctcgagggggctttacctggcggagcgtgttgttctcgttctggagggtggagacgcgctgctcgagctcggcattgcggagctcgaggtccttggccttggcctcgagctccgtcatgtacgccttcttccgctcccgcgcctgctgcgccgacacgcggttccgcagcagccgcttcagccggttctgctccttgtcgccggcgctccgccctcgcttcctcgccggcggcgcctgctcctgcccgccccccgccgccgccgccccgccgccgccaccaccctgctgcttcccgtcctccttcccctgccgctcgtccgcccccgcccccgacgacgccgacccgccgccccctcccatctccggcacccgccgtatctcctcgtcgctctccacccctgccgccaccgaatcgctcgctcaattcagcagcaaacaacaaaacaagcaaaggaaatccggcgtacggacggccgacggagaacgtgacgttacctcctccttccttgaggttgttgggggctgagctggaggagcgctcgctgctcgacggcagcgagctcgtcgtgctcgtcttcacctgctgcttctcctgctcctgctcctgcgccgccatctccaacgaccagatcaagatctcccccaccaaccaccacaccacaccacactcaccctcccccctcgcccctcgccgccgcgaaaaagggaagaaaaaaaaagaaaatcaaatctagaagaagaagaagaaacaagagaccacgacgaacacgaagcacaagtgtggaaaggagaagcagatgcagatcggatgagaggagagagagagaaatcgagagagcggaggagagagaaaacgagtctgtgtgctctgctgcgggatgggaggagagagagagagatggggggaaatgggtaggagaggtcggtggggttggggggttttggagggcgacgtggccgtcatccgggccgtccactccggagccatccgacggtgggggttcggggagcgtggcgtgcgaaggcaccatacacgcatccaccgcatctgacggtgacctccccggaagcgtagcggcatccccatccatccgatttcgtaaaagcgtaaaaccacttgcctttctcggacggaacggaagctgtgagccat

SEQ ID NO: 8 - protein, 223 aa, Oryza sativa
  G4627 polypeptide
MAAQEQEQEKQQVKTSTTSSLPSSSERSSSSAPNNLKEGGGNVTFSVGRPYAGFPLLVLLFAAELSERFGGGRGGERRGDTAGAGDGRGRRVGVVGGGGGRAAGEGGREAAGWWRRRGGGGGGRAGAGAAGEEARAERRRQGAEPAEAAAAEPRVGAAGAGAEEGVHDGARGQGQGPRAPQCRARAARLHPPEREQHAPPGKAPSSFASPFSISHGDFSSTLL

SEQ ID NO: 9 - DNA, 1083 bp, Oryza sativa
  G4630
atggcgacaacacgcgcatctctcaccgatcccctccttccctctcccgcggcacgcgcgccagttaaagccaaaaagctctcatggtccatgcttcacgcaagcagcaaggacgagaggagaggacagagtggggaagctgaagctgaagcaagcggaggagtgcacgcgaatccctcctcgccggcgagaatgcaggagcaggcgacgagctcgcggccgtccagctccgagaggtcgtccagctccggcggccaccacatggagatcaaggaaggcaaggaagcgccacttcgatcccttctccttccctttcttgatttccattttactgttcctctttcgggaatggagagcgacgaggagatagggagagtgccggagctggggctggagccgggcggcgcttcgacgtcggggagggcggccggcggcggcggcggcggggcggagcgcgcgcagtcgtcgacggcgcaggccagcgcgcgccgccgcgggcgcagccccgcggataaggagcacaagcgcctcaaaaggttgctgaggaaccgggtatcagcgcagcaggcaagggagagaaagaaggcatacttgaatgatcttgaggtgaaggtgaaggacttggagaagaagaactcagagttggaagaaagattctccaccctacagaatgagaaccagatgctcagacagatactgaagaatacaactgtgagcagaagagggccagttcttctgaaaatccccaaatcgggtctgcgggaggcggcaccagcgggctgcggaggtttgcgggaggcggagggcgacgagaagtttgtcctcaacgggttcaccgccgcgaatctcagcttcgatggcatggcgacggtgaccccgaacgggctgctcatgttgaccaacggcacgaaccagctcaagggccacgccttcttcccggcgctgctccagttccacaggacgcccaacagcatggcgatgcagtccttctccacggccttcgtcatcggcatcatcagcgcgttcgaggaccagggcagcggcagcccggcggcggcaggtggcagcggcagggcggcataa

SEQ ID NO: 10 - protein, 360 aa, Oryza sativa
  G4630 polypeptide
MATTRASLTDPLLPSPAARAPVKAKKLSWSMLHASSKDERRGQSGEAEAEASGGVHANPSSPARMQEQATSSRPSSSERSSSSGGHHMEIKEGKEAPLRSLLLPFLDFHFTVPLSGMESDEEIGRVPELGLEPGGASTSGRAAGGGGGGAERAQSSTAQASARRRGRSPADKEHKRLKRLLRNRVSAQQARERKKAYLNDLEVKVKDLEKKNSELEERFSTLQNENQMLRQILKNTTVSRRGPVLLKIPKSGLREAAPAGCGGLREAEGDEKFVLNGFTAANLSFDGMATVTPNGLLMLTNGTNQLKGHAFFPALLQFHRTPNSMAMQSFSTAFVIGIISAFEDQGSGSPAAAGGSGRAA

SEQ ID NO: 11 - DNA, 780 bp, Zea mays
  G4632
atcgcaggcagatagggaaggagaagcggagtgcgcgcggtccaaatctgcggaggcggaggcggaggcggagggcgagcaagaatgcaggagcagccggcgagctcgcggccttccagcagcgagaggtcgtctagctccgcgcaccacatggacatggaggtcaaggaagggatggagagcgacgaggagataaggagagtgccggagctgggcctggagctgccgggagcttccacgtcgggcagggaggttggcccgggcgccgccggcgcagaccgcgccctggcccagtcgtccacggcgcaggccagcgcgcgccgccgcgtccgcagccccgccgacaaggagcacaagcgcctcaaaagattactgaggaaccgggtgtcagctcaacaggcaagagagaggaagaaggcttatttgactgatctggaggtgaaggtgaaggacctggagaagaagaactcggagatggaagagaggctctccaccctccagaacgagaaccagatgctccgacagatactgaagaacaccactgtaagcagaagaggttcaggaagcactgctagtggagagggccaatagttcagaatgacaggaaaatagtaatgcattatatgctaaacatatgtttatgctcagtggatttggtcagtttgctttgtggccaaaggagggaaccccaaaaactgggggtgaaggatttgtgcagacagtcatatatatcactgtattaatacgaatggttcagaaaaagaagaacttatggagtgc

SEQ ID NO: 12 - protein, 168 aa, Zea mays
  G4632 polypeptide
MQEQPASSRPSSSERSSSSAHHMDMEVKEGMESDEEIRRVPELGLELPGASTSGREVGPGAAGADRALAQSSTAQASARRRVRSPADKEHKRLKRLLRNRVSAQQARERKKAYLTDLEVKVKDLEKKNSEMEERLSTLQNENQMLRQILKNTTVSRRGSGSTASGEGQ

SEQ ID NO: 13 - DNA, 2331 bp, Arabidopsis thaliana
  G1518 (COP1)
caaaaaccaaaatcacaatcgaagaaatcttttgaaagcaaaatggaagagatttcgacggatccggttgttccagcggtgaaacctgacccgagaacatcttcagttggtgaaggtgctaatcgtcatgaaaatgacgacggaggaagcggcggttctgagattggagcaccggatctggataaagacttgctttgtccgatttgtatgcagattattaaagatgctttcctcacggcttgtggtcatagtttctgctatatgtgtatcatcacacatcttaggaacaagagtgattgtccctgttgtagccaacacctcaccaataatcagctttaccctaatttcttgctcgataagctattgaagaaaacttcagctcggcatgtgtcaaaaactgcatcgcccttggatcagtttcgggaagcactacaaaggggttgtgatgtgtcaattaaggaggttgataatcttctgacacttcttgcggaaaggaagagaaaaatggaacaggaagaagctgagaggaacatgcagatacttttggactttttgcattgtctaaggaagcaaaaagttgatgaactaaatgaggtgcaaactgatctccagtatattaaagaagatataaatgccgttgagagacatagaatagatttataccgagctagggacagatattctgtaaagttgcggatgctcggagatgatccaagcacaagaaatgcatggccacatgagaagaaccagattggtttcaactccaattctctcagcataagaggaggaaattttgtaggcaattatcaaaacaaaaaggtagaggggaaggcacaaggaagctctcatgggctaccaaagaaggatgcgctgagtgggtcagattcgcaaagtttgaatcagtcaactgtctcaattgctagaaagaaacggattcatgctcagttcaatgatttacaagaatgttacctccaaaagcggcgtcagttggcagaccaaccaaatagtaaacaagaaaatgataagagtgtagtacggagggaaggctatagcaacggccttgcagattttcaatctgtgttgactaccttcactcgctacagtcgtctaagagttatagcagaaatccggcatggggatatatttcattcagccaacattgtatcaagcatagagtttgatcgtgatgatgagctgtttgccactgctggtgtttctagatgtataaaggtttttgacttctcttcgtttgtaaatgaaccagcagatatgcagtgtccgattgtggagatgtcaactcggtctaaacttagttgcttgagttggaataagcatgaaaaaaatcacatagcaagcagtgattatgaaggaatagtaacagtgtgggatgtaactactaggcagagtcggatggagtatgaagagcacgaaaaacgtgcctggagtgttgacttttcacgaacagaaccatcaatgcttgtatctggtagtgacgactgcaaggttaaagtttggtgcacgaggcaggaagcaagtgtgattaatattgatatgaaagcaaacatatgttgtgtcaagtacaatcctggctcaagcaactacattgcggtcggatcagctgatcatcacatccattattacgatctaagaaacataagccaaccacttcatgtcttcagtggacacaagaaagcagtttcctatgttaaatttttgtccaacaacgagctcgcttctgcgtccacagatagcacactacgcttatgggatgtcaaagacaacttgccagttcgaacattcagaggacatactaacgagaagaactttgtgggtctcacagtgaacagcgagtatctcgcctgtggaagcgagacaaacgaagtatatgtatatcacaaggaaatcacgagacccgtgacatcgcacagatttggatcgccagacatggacgatgcagaggaagaggcaggttcctactttattagtgcggtttgctggaagagtgatagtcccacgatgttgactgcgaatagtcaaggaaccatcaaagttctggtactcgctgcgtgattctagtagacattacaaaagatcttatagcttcgtgaatcaataaaaacaaatttgccgtctatgttctttagtgggagttacatatagagagagaacaatttattaaaagtagggttcatcatttggaaagcaactttgtattattatgcttgccttggaacactcctcaagaagaatttgtatcagtgatgtagatatgtcttacggtttcttagcttctactttatataattaaatgttagaatcaaaaaaaaaaaa

SEQ ID NO: 14 - protein, 616 aa, Arabidopsis thaliana
  G1518 (COP1) polypeptide
MEEISTDPVVPAVKPDPRTSSVGEGANRHENDDGGSGGSEIGAPDLDKDLLCPICMQIIKDAFLTACGHSFCYMCIITHLRNKSDCPCCSQHLTNNQLYPNFLLDKLLKKTSARHVSKTASPLDQFREALQRGCDVSIKEVDNLLTLLAERKRKMEQEEAERNMQILLDFLHCLRKQKVDELNEVQTDLQYIKEDINAVERHRIDLYRARDRYSVKLRMLGDDPSTRNAWPHEKNQIGFNSNSLSIRGGNFVGNYQNKKVEGKAQGSSHGLPKKDALSGSDSQSLNQSTVSMARKKRIHAQFNDLQECYLQKRRQLADQPNSKQENDKSVVRREGYSNGLADFQSVLTTFTRYSRLRVIAEIRHGDIFHSANIVSSIEFDRDDELFATAGVSRCIKVFDFSSVVNEPADMQCPIVEMSTRSKLSCLSWNKHEKNHIASSDYEGIVTVWDVTTRQSLMEYEEHEKRAWSVDFSRTEPSMLVSGSDDCKVKVWCTRQEASVINIDMKANICCVKYNPGSSNYIAVGSADHHIHYYDLRNISQPLHVFSGHKKAVSYVKFLSNNELASASTDSTLRLWDVKDNLPVRTFRGHTNEKNFVGLTVNSEYLACGSETNEVYV

SEQ ID NO: 15 - DNA, 2731 bp, Glycine max
  misc_feature (2724)..(2724): n is a, c, g, or t
attcggctcgagaccccaattccgaagcaaaaactaccttcacatccacaaaccacacctccgccataaataaaagtaacctccctcatggaagagctctcagcggggcctctcgtccccgccgtcgtcaaacctgaaccgtccaaaggcgcctccgccgctgcctccggcggcacgttcccggcctccacgtcggagccggacaaggacttcctctgtccgatttgcatgcagatcatcaaggacccgttcctcaccgcgtgcggccacagcttctgctacatgtgcatcatcacgcacctccgcaacaagagcgattgcccttgctgcggcgactacctcaccaacaccaacctcttccctaacttgttgctcgacaagcttattgttatacggtttctgtaccacatttgtagctactgaagaagacttctgcgcgtcaaatatcaaaaaccgcttcacctgtcgaacattttcggcaggtattgcaaaagggttctgatgtgtcaattaaggagctagacacccttttgtcacttcttgccgagaagaaaagaaaaatggaacaagaagaagctgagagaaatatgcaaatattgttagacttcttgcattgcttacgcaagcaaaaagttgatgagttgaaggaggtacaaactgatctccactttataaaagaggacataaatgctgtggagaaacatagaatggaattgtatcgtgcacgggacaggtactctgtaaaattgcagatgcttgacggttctgggggaagaaaatcatggcattcatcaatggacaagaacagcagtggctacggctgcgagaagacgacagaagggggagggttgtcatcagggagccatactaagaaaaatgatggaaagtctcatattagctctcatgggcatggaattcagagaaggaatgtcatcactggatccgattcacaatatataaatcaatcgggtcttgctctagttagaaagaagagggtgcatacacagttcaatgatctacaagaatgttacctacaaaagcgacggcatgcagctgataggtcccatagccaacaagaaagagatataagtctcataagtcgagaaggttatactgctggtcttgaagattttcagtcagtcttgacaactttcacacgctatagccgattgagagtcattgcagaactaagacatggggatatatttcattcagcaaatatagtgtcaagcatagagtttgactgcgatgatgatttgtttgctactgctggagtttcccggcgcatcaaagtttttgacttttctgctgttgtgaatgaacctacagatgctcactgtcctgttgtggagatgtctacacgttcaaaacttagttgcttgagttggaataaatatgctaagaatcaaatagctagtagtgattatgaaggaattgtgactgtttgggatgtaaccactcgaaagagtttaatggaatatgaagagcatgaaaagcgtgcatggagtgttgatttttcaagaacagatccctctatgcttgtatctggtagcgatgactgtaaggtcaaaatttggtgtacaaatcaggaagctagtgttctaaatatagacatgaaagcaaacatatgctgtgtcaaatataatcctggatctggcaattatattgcagttggatcagcagaccatcacatccattattatgatttgagaaatattagccgtccagtccatgttttcagtgggcacaggaaggctgtttcatacgtgaaatttctgtctaatgatgaacttgcttctgcatcaacagatagtacactgcgattatgggatgtgaaggaaaacttaccagttcgtactttcaaaggccatgcaaatgagaaaaactttgttggtcttacagtaagcagtgaatacattgcgtgtggcagtgaaacaaatgaagtctttgtgtaccacaaggaaatctcgagacctttgacttgccacagatttgggtcccctgatatggatgacgctgaagatgaggctggatcgtacttcattagtgctgtatgctggaagagtgatcgccccactattctaactgcaaatagtcaaggcaccatcaaagtgctggtgcttgcagcttgaacacgagaaaaaagaatagaatgtggaattggtattatcttttcccatgctattatgattgtatcatttattaattgtacatagttttcaagtgtatatggcaggctttagggatcttaatgagatattagttgagtgcttaaacctttatcaacaaacctatttaagggactgaactttaatttttaccaattgaggacctcaaatttattaaattttgtattaataaatgctcaggagacaaaataaaatatcaaatttggcatgtgataataatgataatatcagcaaagcacctagtgtatatgatttaactttttaaatacataactatgattgttactattgtgttaaaattgaggtcctcaattgatattgaaataagttaaggttcttaacataaattttgaagttaaagtcttccttaattggttataacattatagttaaggtccttcgagtacaaacttgttgaggttactcttcatattgtcatttccaaggaaacacgtgtattaattttttatcattggttgtttcggagagaaaaaaaaatgtttttgttctgctccttgattgccatctttactanattgaga

SEQ ID NO: 16 - protein, 643 aa, Glycine max
  G4633 polypeptide
MEELSAGPLVPAVVKPEPSKGASAAASGGTFPASTSEPDKDFLCPICMQIIKDPFLTACGHSFCYMCIITHLRNKSDCPCCGDYLTNTNLFPNLLLDKLLKKTSARQISKTASPVEHFRQVLQKGSDVIKELDTLLSLLAEKKRKMEEEAERNMETQILLDFLHCLRKKVDELKEVQTDLHFIKEDIAVEKHRMELYRARDRYSVKQMLDGSGGRKSWHSSMDKNSGYGCEKTTEGGGLSSGSHKKNDGKSHISSHGHGIQRRVITGSDSQYINQSGLALVRKRVHTQFNDLQECYLQKRRAADRSHSQQERDISLISREYTAGLEDFQSVLTTFTRYSLRVIAELRHGDIFHSANIVSIEFDCDDDLFATAGVSRRKVFDFSAVVNEPTDAHCPVEMSTRSKLSCLSWNKYAKNIASSDYEGIVTVWDVTTRKLMEYEEHEKRAWSVDFSRTPSMLVSGSDDCKVKIWCTNEASVLNIDMKANICCVKYNGSGNYIAVGSADHHIHYYDRNISRPVHVFSGHRKAVSYKFLSNDELASASTDSTLRLDVKENLPVRTFKGHANEKNVGLTVSSEYIACGSETNEVVYHKEISRPLTCHRFGSPDDDAEDEAGSYFISAVCWKSRPTILTANSQGTIKVLVLA

SEQ ID NO: 17 - DNA, 2434 bp, Oryza sativa
  G4628
ttattcacgcccagtcgccgcctccaccgccgccgcctgctcgactcaccaccgcagggcggcctcctcctgccgcatgggtgactcgacggtggccggcgcgctggtgccatcggtgccgaagcaggagcaggcgccgtcgggggacgcgtccacggcggcgttggcggtggcgggggagggggaggaggatgcgggggcgcgcgcctccgcggggggcaacggggaggccgcggccgacagggacctcctctgcccgatctgcatggcggtcatcaaggacgccttcctcaccgcctgcggccacagcttctgctacatgtgcatcgtcacgcatctcagccacaagagcgactgcccctgctgcggcaactacctcaccaaggcgcagctctaccccaacttcctcctcgacaaggtcttgaagaaaatgtcagctcgccaaattgcgaagacagcatcaccgatagaccaatttcgatatgcactgcaacagggaaacgatatggcggttaaagaactagatagtcttatgactttgatcgcggagaagaagcggcatatggaacagcaagagtcagaaacaaatatgcaaatattgctggtcttcttgcattgcctcagaaagcaaaagttggaagagctgaatgagattcaaactgacctacagtacatcaaagaagatataagtgctgtggagagacataggttagaattatatcgaacaaaagaaaggtactcaatgaagctccgcatgcttttggatgaacctgctgcatcaaagatgtggccttcacctatggataaacctagtggtctctttcttcccaactctcggggaccacttagtacatcaaatccagggggtttacagaataagaagcttgacttgaaaggtcaaattagtcatcaaggatttcaaaggagagatgttctcacttgctcggatcctcctagtgcccctattcaatcaggcaacgttattgctcggaagaggcgagttcaagctcagtttaacgagcttcaagaatactatcttcaaagacggcgtaccggagcacaatcacgtaggctggaggaaagagacatagtaacaataaataaagaaggttatcatgcaggacttgaggatttccagtctgtgctaacaacattcacacgatatagtcgcttgcgtgtaattgcggagctaagacatggagatctgtttcactctgcaaatatcgtatcaagtatcgaatttgaccgtgatgatgagctatttgctactgctggagtctcaaagcgcatcaaagtcttcgagttttctacagttgttaatgaaccatcagatgtgcattgtccagttgttgaaatggctactagatctaaactcagctgccttagctggaacaagtactcaaaaaatgttatagcaagcagcgactatgagggtatagtaactgtttgggatgtccaaacccgccagagtgtgatggagtatgaagaacatgaaaagagagcatggagtgttgatttttctcgaacagaaccctcgatgctagtatctgggagtgatgattgcaaggtcaaagtgtggtgcacaaagcaagaagcaagtgccatcaatattgatatgaaggccaatatttgctctgtcaaatataatcctgggtcgagccactatgttgcagtgggttctgctgatcaccatattcattattttgatttgcgaaatccaagtgcgcctgtccatgtttttggtgggcacaagaaagctgtttcttatgtgaagttcctgtccaccaatgagcttgcgtctgcatcaactgatagcacattacggttatgggatgtcaaagaaaattgccctgtaaggacattcagagggcacaagaatgaaaagaactttgttgggctgtctgtaaataacgagtacattgcctgcgggagtgaaacgaatgaggtttttgtttaccacaaggctatctcaaaacctgctgccaaccacagatttgtatcatctgatctcgatgatgcagatgatgatcctggctcttattttattagcgcagtctgctggaagagcgatagccctaccatgttaactgctaacagtcagggcaccattaaagttcttgtacttgctccttgatgaaatcagtggttttcatgagatccctagatagcttgtatatttgatgtatacagttgtttccttttcgtgccattataccccaaatgggagtggaggtattactgatctccaacatagggcgcaaagttttgaaggtaatcagctgacatagggtttcgagggctcgaaatgtgcatagtccagaattctcatgtataggtttaaagcagtcaagtaattgattatacatatgtaacgtgagaattgagaaatgaacatcaaataagcttgtttggttgcataaaaaaaaaaaaaaaa

SEQ ID NO: 18 - protein, 685 aa, Oryza sativa
  G4628 polypeptide
MGDSTVAGALVPSVPKQEQAPSGDASTAALAVAGEGEEDAGARASAGGNGEAAADRDLLCPICMAVIKDAFLTACGHSFCYMCIVTHLSHKSDCPCCGNYLTKAQLYPNFLLDKVLKKMSARQIAKTASPIDQFRYALQQGNDMAVKELDSLMTLIAEKKRHMEQQESETNMQILLVFLHCLRKQKLEELNEIQTDLQYIKEDISAVERHRLELYRTKERYSMKLRMLLDEPAASKMWPSPMDKPSGLFLPNSRGPLSTSNPGGLQNKKLDLKGQISHQGFQRRDVLTCSDPPSAPIQSGNVIARKRRVQAQFNELQEYYLQRRRTGAQSRRLEERDIVTINKEGYHAGLEDFQSVLTTFTRYSRLRVIAELRHGDLFHSANIVSSIEFDRDDELFATAGVSKRIKVFEFSTVVNEPSDVHCPVVEMATRSKLSCLSWNKYSKNVIASSDYEGIVTVWDVQTRQSVMEYEEHEKRAWSVDFSRTEPSMLVSGSDDCKVKVWCTKQEASAINIDMKANICSVKYNPGSSHYVAVGSADHHIHYFDLRNPSAPVHVFGGHKKAVSYVKFLSTNELASASTDSTLRLWDVKENCPVRTFRGHKNEKNFVGLSVNNEYIACGSETNEVFVYHKAISKPAANHRFVSSDLDDADDDPGSYFISAVCWKSDSPTMLTANSQGTIKVLVLAP

SEQ ID NO: 19 - DNA, 2871 bp, Pisum sativum
  G4629
ggcacgaggcggccgctcctggctcaggatgaacgctggcggcatgctttacacatgcaagtcggacgggaagtggtgtttccagtggcgaacgggtgagtaacgcgtaaaaacctgcccttgggagggggacaacagctggaaacggctgctaataccccgtaggctgaggagcgaaaggaggaatccgcccaaggaggggctcgcgtctgattagctagttggtgaggtaataccttaccaaggcaatgatcagtacctggtccgaaaggatgatcagccacactggggactgagacaaggtccaaactcctacgggaggcagcagtggggaattttccgcaatgggcgaaagcctgacggagcaatgccccgtggaggtagaggcccctgggtcatgaacttcttttcccggagaagaaaaaatgacggtatccggggaataagcatcggctaactctgtgccagcagccgcggtaagacagaggatgcaagcgttatccggaatgattgggcgtaaagcgtctgtaggtggctttttaagttcgctgtcaaataccagggctcaaccctggacaggtggtgaaaaccacatccactctaaacctcaccatggaagagcactcagtaggacctctagtccctgcagtagtgaaaccagaaccttccaaaaacttctccaccgacaccaccgccgccggcacgtttctcctggttcccaccatgtctgacctagataaggacttcctctgcccgatttgcatgcagatcatcaaagacgcgtttctcacagcctgtggtcatagcttctgctacatgtgtatcatcactcatctccgtaacaaaagcgattgtccttgctgtggtcattacctcaccaacagtaatttgttcccgaacttcctgctcgataagctactaaaaaagacatcagatcgtcaaatatcaaagacggcttctcctgtggagcatttccggcaggcagtacaaaagggctgtgaagtgacaatgaaggagctcgacacccttttgttactccttactgagaagaaaagaaaaatggaacaagaagaagctgagagaaatatgcaaatattgttagatttcttgcattgcctacgcaagcaaaaagttgatgagttgaaggaggtgcaaactgatctccagttcataaaggaggacattggtgctgtggagaaacatagaatggatttgtatcgtgctcgagacaggtactctgtgaaattgcggatgcttgacgattctggtggaagaaaatcacggcattcatcaatggacttgaatagcagtggcctcgcatctagtcctttaaatcttcgaggagggttatcttcagggagccatactaagaaaaatgatggaaagtcacaaatcagctctcatgggcatggaattcagagaagagatcccatcactggatcagattcacagtatataaatcaatcgggtcttgctctagttagaaagaaaagggtgcatacacagttcaatgacctacaagaatgttatctacaaaaacgacggcaagcagcagataagccacatggccaacaggaaagggatacaaatttcataagtcgagaaggttatagctgtggtcttgatgattttcagtcagtcttgacaactttcacacgctacagccgattgagagtcattgcagaaataagacacggggatatatttcattcagccaacattgtttcaagcatagagtttgaccgtgatgatgatttgtttgctactgctggagtttcccgacgtatcaaagtttttgatttttctgcggtcgtgaatgaacccacagatgctcattgtcctgttgtggagatgactacacgttcaaaacttagttgcttgagttggaacaaatatgctaagaaccaaatagctagtagtgattatgaaggaattgtaactgtttggacgatgaccactcgaaagagtttaatggaatatgaagagcatgaaaagcgtgcatggagtgttgatttttcaagaacggacccctctatgcttgtatctggtagtgatgattgtaaggtcaaagtttggtgcacaaatcaggaggccagtgttctaaatatagacatgaaagcaaacatatgctgcgtgaagtataatcctggatctgggaattacatcgcagttgggtctgcagaccatcacatccattattatgatttgagaaatattagccggccagtccatgttttcactgggcacaagaaggctgtttcatacgtgaaatttttgtccaacgatgaacttgcatcggcatcaacagatagtacactgcggttatgggatgtaaagcaaaacttaccagttcgtaccttcagaggccacgcaaatgagaaaaactttgttggccttacagttcgcagtgagtacattgcatgtggcagtgaaacaaatgaagtatttgtctaccacaaggaaatttctaagcctctgacatggcatagatttggtaccttagacatggaagacgcggaggatgaggctggatcttacttcatcagtgctgtatgctggaagagtgatcgccccaccatactaactgcaaatagtcaaggcaccatcaaagtgctggtgcttgctgcttaaatacaagaaaaaatgaacagaatgctgaatcgggattggttgttcctatgctacaaattggtgtaccattaaaattgtacagagtatcgaagtgtatatgataggttttagggatctcattgaggtattagctgaggatactatatgatccaatcaattaagaaactgaacttttgccaattaaggatctcaagtttaataaaataaattagttttaggattaaaaaaaaaaaaaaaaaa

SEQ ID NO: 20 - protein, 672 aa, Pisum sativum
  G4629 polypeptide
MEEHSVGPLVPAVVKPEPSKNFSTDTTAAGTFLLVPTMSDLDKDFLCPICMQIIKDAFLTACGHSFCYMCIITHLRNKSDCPCCGHYLTNSNLFPNFLLDKLLKKTSDRQISKTASPVEHFRQAVQKGCEVTMKELDTLLLLLTEKKRKMEQEEAERNMQILLDFLHCLRKQKVDELKEVQTDLQFIKEDIGAVEKHRMDLYRARDRYSVKLRMLDDSGGRKSRHSSMDLNSSGLASSPLNLRGGLSSGSHTKKNDGKSQISSHGHGIQRRDPITGSDSQYINQSGLALVRKKRVHTQFNDLQECYLQKRRQAADKPHGQQERDTNFISREGYSCGLDDFQSVLTTFTRYSRLRVIAEIRHGDIFHSANIVSSIEFDRDDDLFATAGVSRRIKVFDFSAVVNEPTDAHCPVVEMTTRSKLSCLSWNKYAKNQIASSDYEGIVTVWTMTTRKSLMEYEEHEKRAWSVDFSRTDPSMLVSGSDDCKVKVWCTNQEASVLNIDMKANICCVKYNPGSGNYIAVGSADHHIHYYDLRNISRPVHVFTGHKKAVSYVKFLSNDELASASTDSTLRLWDVKQNLPVRTFRGHANEKNFVGLTVRSEYIACGSETNEVFVYHKEISKPLTWHRFGTLDMEDAEDEAGSYFISAVCWKSDRPTILTANSQGTIKVLVLAA

SEQ ID NO: 21 - DNA, 2373 bp, Solanum lycopersicum
  G4635
atacccaatttgcatttgggggtatagagggagatggtggaaagttcagttggaggggtggtgccagcagtgaagggggaggtgatgaggaggatgggggacaaagaggaggggggtagtgtaactctaagggatgaagaagttgggacagtgacagaatgggaattggacagggaattgttgtgtcctatatgtatgcagatcataaaggatgcatttttaacagcttgtgggcacagtttttgctatatgtgcatagttactcatcttcacaacaagagtgattgcccctgttgttctcattatctcactaccagtcaactctatcccaatttcctacttgacaagctattgaagaagacatctgcccgtcagatttcaaaaactgcatcccctgttgaacagtttcgtcattcattggaacagggttctgaagtgtcaattaaggagctggacgctctattgttgatgttgtcagagaaaaagaggaaattggaacaggaggaagcagagcgaaatatgcaaattctgctagacttcttacagatgttaaggaagcaaaaagttgatgaactcaatgaggtgcaacatgatctgcaatacatcaaagaggacttaaattcagtagagagacatagaatagacctataccgggctagggaccggtattcaatgaagctccgaatgttagcagatgatcctattgggaaaaaaccttggtcttcatcaactgataggaactttggtggtcttttctccacttcacaaaatgcacctggaggattaccgactggaaacttgacattcaaaaaggtggacagcaaagctcaaataagctctcctggaccacagagaaaagatacttcaatcagtgaactgaactcacaacatatgagtcaatcaggtctggctgtggttaggaagaagcgtgtcaatgcacagttcaatgatctccaagaatgttacttgcaaaagagacgtcaattggcaaacaaatcgcgagttaaggaagaaaaggatgcagatgtcgtacaaagagaaggttacagtgaaggactagcagattttcagtctgtacttagcactttcactcgttatagtcggttaagagtcattgctgaacttcggcatggggatctgtttcactcggccaatattgtttcaagcattgaatttgatcgggatgatgagttgtttgctactgctggagtttcacggcgtataaaagtttttgacttctcttcagttgtaaatgaacctgcagatgcacactgccctgttgttgaaatgtctacccgatctaagctgagctgcttgagttggaataagtataccaagaaccacatagctagtagtgattatgatggaatagtaactgtatgggatgtgacgactagacagagtgtgatggaatatgaagagcatgagaaacgggcttggagtgttgatttttcacgcacagaaccctcgatgcttgtatctggcagtgatgattgtaaggtcaaagtttggtgcacgaagcaggaagcaagtgttcttaatattgacatgaaggcaaatatatgctgtgtaaaatataatcctggatctagtgttcatatagcggttggctctgcggatcatcatattcattattatgacttgaggaacaccagccagccggttcacatttttagtggccatagaaaagctgtttcatatgtaaaatttttgtccaacaatgaacttgcttcagcatcaacagacagtactctacgattgtgggatgtaaaagataatttgccggttcgcacgcttagaggacatacgaatgagaagaactttgttggtctctcagtgaacaatgaattcctgtcatgtggcagtgaaacaaatgaagtattcgtgtaccataaggcgatatccaaacccgtgacttggcatagatttggttccccagacatagacgaagcggatgaagatgcaggatcttatttcatcagcgcagtgtgctggaagagcgatagccctacgatgctagctgctaatagccagggaactataaaagtgttagtccttgcagcttgatgaagttaataaagctactagttaagaatgttcaaatctttttagtggaaaaacagtgaaatggaatttcacattcaatttttcctgtagatatctattcaaccatcaagatggcatggttccccccatatttgtcaatgtattcatcattaaaacatgtaacacaagttgtagggcttggtaaatttagaagaattttacaagtttgtgttttttttttcattgtgctgaaggacatcggatttacacaccatttcatggaataaactttactcgtattcagtgtttaaaaaaaaaaaaaaaaaa

SEQ ID NO: 22 - protein, 677 aa, Solanum lycopersicum
  G4635 polypeptide
MVESSVGGVVPAVKGEVMRRMGDKEEGGSVTLRDEEVGTVTEWELDRELLCPICMQIIKDAFLTACGHSFCYMCIVTHLHNKSDCPCCSHYLTTSQLYPNFLLDKLLKKTSARQISKTASPVEQFRHSLEQGSEVSIKELDALLLMLSEKKRKLEQEEAERNMQILLDFLQMLRKQKVDELNEVQHDLQYIKEDLNSVERHRIDLYRARDRYSMKLRMLADDPIGKKPWSSSTDRNFGGLFSTSQNAPGGLPTGNLTFKKVDSKAQISSPGPQRKDTSISELNSQHMSQSGLAVVRKKRVNAQFNDLQECYLQKRRQLANKSRVKEEKDADVVQREGYSEGLADFQSVLSTFTRYSRLRVIAELRHGDLFHSANIVSSIEFDRDDELFATAGVSRRIKVFDFSSVVNEPADAHCPVVEMSTRSKLSCLSWNKYTKNHIASSDYDGIVTVWDVTTRQSVMEYEEHEKRAWSVDFSRTEPSMLVSGSDDCKVKVWCTKQEASVLNIDMKANICCVKYNPGSSVHIAVGSADHHIHYYDLRNTSQPVHIFSGHRKAVSYVKFLSNNELASASTDSTLRLWDVKDNLPVRTLRGHTNEKNFVGLSVNNEFLSCGSETNEVFVYHKAISKPVTWHRFGSPDIDEADEDAGSYFISAVCWKSDSPTMLAANSQGTIKVLVLAA

SEQ ID NO: 23 - DNA, 1340 bp, Arabidopsis thaliana
  G1482 (STH2)
ttaccagaaagatctaaactttttattagaagaaagaggaggaggagtgatctgtgggacagtgaagccaccatcatcataccatctcttgttgttctgtccttgttgtttcatgttttgtattggagcaaaagacactacttctggtgatgtttctttgttgtacatcccaaactgtatgttgttgtcttgagaaaagtattgatttgggtatgaagaaggaagagtttgtggaatctgagggacccaaatccctaaattcttagatggaagtgacactgtattgttgttgttgttgttgttgttgttgttgtttctcttagtgttgttgtcatcttctggttccatatatggtaacactccatcatcatcaccactctgcaatcacacaaaagataaccaacaactctttttcagaaattttacacaaatacccaatatagtaaaaagatctatccacatctataaagtttgttacctttataatacattaatacctcattagatctaaaatgatatgatattacgtaaacagaggaaaaaaaaattcaatctactaagggtcattgtcaaatcttgaaatcaactaaacttggatctttcttgattaaagagataagaacaaaccttagagaaaccataagtaggaagagaggaatcgaggaaatcctcaacgtgccaaccaggtaacgtatccatcaaatactcagaaatcgtgcttgtggatccccactgattcaccgacgcatcaccgccgttgatcttcgaaaagggttggatcttgttgctctgaggaggagctgagagaggtttcttgagaggaggaggattagagattgatgatccagggacagagaaatcttggttgcttgaagaagaagaagaagatttcgaagtaggtttgtaaacagacgatgttgcagagagcttaacccctgtaagaagaaacctatcgtgtttctttgtgtgttcgttcgcagcgtggatcgatgaatcgcaatctttgcataaaatagctctatcttgttgacagaacaacagagcttttttatcctagagttcaataaaaagaaaaagtttcagattcttgatcggcaaaaacgattgaattaagacaacaaaactcatgtccgaagttagaaagagacctgacagatgtcgcagagaggagaggaggtgttggaagaagaaggataaaggagagagaaacggagatgtttagaggcgagtttgttagcgtggtggacttggtggtcgcagccgccgcagagagatgcttcgtcggccgtgcaaaacaccgacgcttcttctttatcgcagacgtcgcacctgatcttcat

SEQ ID NO: 24 - protein, 331 aa, Arabidopsis thaliana
  G1482 (STH2) polypeptide
MKIRCDVCDKEEASVFCTADEASLCGGCDHQVHHANKLASKHLRFSLLYPSSSNTSSPLCDICQDKKALLFCQQDRAILCKDCDSSIHAANEHTKKHDRFLLTGVKLSATSSVYKPTSKSSSSSSSNQDFSVPGSSISNPPPLKKPLSAPPQSNKIQPFSKINGGDASVNQWGSTSTISEYLMDTLPGWHVEDFLDSSLPTYGFSKSGDDDGVLPYMEPEDDNNTKRNNNNNNNNNNNTVSLPSKNLGIWVPQIPQTLPSSYPNQYFSQDNNIQFGMYNKETSPEVVSFAPIQNMKQQGQNNKRWYDDGGFTVPQITPPPLSSNKKFRSFW

SEQ ID NO: 25 - DNA, 729 bp, Arabidopsis thaliana
  G1888
atgaagatttggtgtgctgtttgtgataaagaagaagcttcggtgttttgttgtgcggatgaagcagctctttgtaatggttgcgatcgccatgttcatttcgccaataaactagccgggaaacatctccggttctctctcacttctcctactttcaaagatgctcctctttgtgatatttgcggggagaggcgtgcattattattttgccaagaagacagagcaatactatgcagagaatgtgacattccaatacatcaagctaatgagcacactaagaaacacaatagattcctccttaccggcgttaagatctctgcctccccgtcagcctacccaagagcctccaattccaactctgctgctgcatttggtcgagccaaaacccgaccaaaatcagtatcgagcgaggtcccgagctcggcctccaatgaggtatttacgagctcttcttcgacgaccacgagcaattgctattatgggatagaagaaaactaccatcacgtgagcgattcggggtcgggatcgggttgtacaggtagtatatccgagtatttgatggagacattaccgggttggagagtggaggatttgcttgaacacccttcttgtgtctcctatgaggataacattattactaataacaataacagtgagtcttatagggtttatgatggttcttcacaattccatcatcaagggttttgggatcacaaacccttctcttga

SEQ ID NO: 26 - protein, 242 aa, Arabidopsis thaliana
  G1888 polypeptide
MKIWCAVCDKEEASVFCCADEAALCNGCDRHVHFANKLAGKHLRFSLTSPTFKDAPLCDICGERRALLFCQEDRAILCRECDIPIHQANEHTKKHNRFLLTGVKISASPSAYPRASNSNSAAAFGRAKTRPKSVSSEVPSSASNEVFTSSSSTTTSNCYYGIEENYHHVSDSGSGSGCTGSISEYLMETLPGWRVEDLLEHPSCVSYEDNIITNNNNSESYRVYDGSSQFHHQGFWDHKPFS

SEQ ID NO: 27 - DNA, 906 bp, Arabidopsis thaliana
  G1988
tgctactctcatcaaccatgaaccataaaaactccaccgctctttctctccctcaatcatttacatctcttccttaaatctctcttcccaccatcatcattccaaaccaattctctctcacttctttctggtgatcagagagatcgactcaatggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttgagaaatattattgacatgggtcccgcattatgcaaattaggacatttagtgtttagtgcattaattatagtttgtgtc

SEQ ID NO: 28 - protein, 225 aa, Arabidopsis thaliana
  G1988 polypeptide
MVSFCELCGAEADLHCAADSAFLCRSCDAKFHASNFLFARHFRRVICPNCKSLTQNFVSGPLLPWPPRTTCCSESSSSSCCSSLDCVSSSELSSTTRDVNRARGRENRVNAKAVAVTVADGIFVNWCGKLGLNRDLTNAVVSYASLALAVETRPRATKRVFLAAAFWFGVKNTTTWQNLKKVEDVTGVSAGMIRAVESKLARAMTQQLRRWRVDSEEGWAENDNV

SEQ ID NO: 29 - DNA, 732 bp, Glycine max
  G4004
atgaagcccaagacttgcgagctttgtcatcaactagcttctctctattgtccctccgattccgcatttctctgcttccactgcgacgccgccgtccacgccgccaacttcctcgtagctcgccacctccgccgcctcctctgctccaaatgcaaccgtttcgccgcaattcacatctccggtgctatatcccgccacctctcctccacctgcacctcttgctccctggagattccttccgccgactccgattctctcccttcctcttctacctgcgtctccagttccgagtcttgctctacgaatcagattaaggcggagaagaagaggaggaggaggaggaggagtttctcgagttcctccgtgaccgacgacgcatctccggcggcgaagaagcggcggagaaatggcggatcggtggcggaggtgtttgagaaatggagcagagagatagggttagggttaggggtgaacggaaatcgcgtggcgtcgaacgctctgagtgtgtgcctcggaaagtggaggtcgcttccgttcagggtggctgctgcgacgtcgttttggttggggctgagattttgtggggacagaggcctcgccacgtgtcagaatctggcgaggttggaggcaatatctggagtgccagcaaagctgattctgggcgcacatgccaacctcgcacgtgtcttcacgcaccgccgcgaattgcaggaaggatggggcgagtcctag

SEQ ID NO: 30 - protein, 243 aa, Glycine max
  G4004 polypeptide
MKPKTCELCHQLASLYCPSDSAFLCFHCDAAVHAANFLVARHLRRLLCSKCNRFAAIHISGAISRHLSSTCTSCSLEIPSADSDSLPSSSTCVSSSESCSTNQIKAEKKRRRRRRSFSSSSVTDDASPAAKKRRRNGGSVAEVFEKWSREIGLGLGVNGNRVASNALSVCLGKWRSLPFRVAAATSFWLGLRFCGDRGLATCQNLARLEAISGVPAKLILGAHANLARVFTHRRELQEGWGES

SEQ ID NO: 31 - DNA, 756 bp, Glycine max
  G4005
aggcgaagatgaagggtaagacttgcgagctttgtgatcaacaagcttctctctattgtccctccgattccgcatttctctgctccgactgcgacgccgccgtgcacgccgccaactttctcgtagctcgtcacctccgccgcctcctctgctccaaatgcaaccgtttcgccggatttcacatctcctccggcgctatatcccgccacctctcgtccacctgcagctcttgctccccggagaatccttccgctgactactccgattctctcccttcctcttctacctgcgtctccagttccgagtcttgctccacgaagcagattaaggtggagaagaagaggagttggtcgggttcctccgtgaccgacgacgcatctccggcggcgaagaagcggcagaggagtggaggatcggaggaggtgtttgagaaatggagcagagagatagggttagggttagggttaggggtaaacggaaatcgcgtggcgtcgaacgctctgagtgtgtgcctgggaaagtggaggtggcttccgttcagggtggctgctgcgacgtcgttttggttggggctgagattttgtggggacagagggctggcctcgtgtcagaatctggcgaggttggaggcaatatccggagtgccagttaagctgattctggccgcacatggcgacctggcacgtgtcttcacgcaccgccgcgaattgcaggaaggatggggcgagtcctagctagctccaatgtgtaatcgtc

SEQ ID NO: 32 - protein, 241 aa, Glycine max
  G4005 polypeptide
MKGKTCELCDQQASLYCPSDSAFLCSDCDAAVHAANFLVARHLRRLLCSKCNRFAGFHISSGAISRHLSSTCSSCSPENPSADYSDSLPSSSTCVSSSESCSTKQIKVEKKRSWSGSSVTDDASPAAKKRQRSGGSEEVFEKWSREIGLGLGLGVNGNRVASNALSVCLGKWRWLPFRVAAATSFWLGLRFCGDRGLASCQNLARLEAISGVPVKLILAAHGDLARVFTHRRELQEGWGES

SEQ ID NO: 33 - DNA, 726 bp, Oryza sativa
  G4011
atgggtggcgaggcggagcggtgcgcgctctgtggcgcggcggcggcggtgcactgcgaggcggacgcggcgttcctgtgcgcggcgtgcgacgccaaggtgcacggggcgaacttcctcgcgtcgcggcaccaccggaggcgggtggcggccggggcggtggtggtggtggaggtggaggaggaggaggggtatgagtccggggcgtcggcggcgtcgagcacgtcgtgcgtgtcgacggccgactccgacgtggcggcgtcggcggcggcgaggcgggggaggaggaggaggccgagggcagcggcgcggccccgcgcggaggtggttctcgaggggtggggcaagcggatgggcctcgcggcgggggcggcgcggcggcgcgccgcggcggccgggcgcgcgctccgggcgtgcggcggggacgtcgccgccgcgcgcgtcccgctccgcgtcgccatggcggccgcgctgtggtgggaggtggcggcccaccgcgtctccggcgtctccggcgccggccatgccgacgcgctgcggcggctggaggcgtgcgcgcacgtgccggcgaggctgctcacggcggtggcgtcgtcgatggcccgcgcgcgcgcaaggcggcgcgccgccgcggacaacgaggagggctgggacgagtgctcgtgttctgaagcgcccaacgccttgggtggcccacatgtcagtgacacagctcgtcagaaatga

SEQ ID NO: 34 - protein, 241 aa, Oryza sativa
  G4011 polypeptide
MGGEAERCALCGAAAAVHCEADAAFLCAACDAKVHGANFLASRHHRRRVAAGAVVVVEVEEEEGYESGASAASSTSCVSTADSDVAASAAARRGRRRRPRAAARPRAEVVLEGWGKRMGLAAGAARRRAAAAGRALRACGGDVAAARVPLRVAMAAALWWEVAAHRVSGVSGAGHADALRRLEACAHVPARLLTAVASSMARARARRRAAADNEEGWDECSCSEAPNALGGPHVSDTARQK

SEQ ID NO: 35 - DNA, 666 bp, Oryza sativa
  G4012
atggaggtcggcaacggcaagtgcggcggtggtggcgccgggtgcgagctgtgcgggggcgtggccgcggtgcactgcgccgctgactccgcgtttctttgcttggtatgtgacgacaaggtgcacggcgccaacttcctcgcgtccaggcaccgccgccgccggttgggggttgaggtggtggatgaggaggatgacgcccggtccacggcgtcgagctcgtgcgtgtcgacggcggactccgcgtcgtccacggcggcggcggctgcgctggagagcgaggacgtcaggaggagggggcggcgcgggcggcgtgccccgcgcgcggaggcggttctggaggggtgggcgaagcggatggggttgtcgtcgggcgcggcgcgcaggcgcgccgccgcggccggggcggcgctccgcgcggtgggccgtggcgtcgccgcctcccgcgtcccgatccgcgtcgcgatggccgccgcgctctggtcggaggtcgcctcctcctcctcccgtcgccgccgccgccccggcgccggacaggccgcgctgctcctgcggctggaggccagcgcgcacgtgccggcgaggctgctcctgacggtggcgtcgtggatggcgcgcgcgtcgacgccgcccgccgccgaggagggctgggccgagtgctcctga

SEQ ID NO: 36 - protein, 221 aa, Oryza sativa
  G4012 polypeptide
MEVGNGKCGGGGAGCELCGGVAAVHCAADSAFLCLVCDDKVHGANFLASRHRRRRLGVEVVDEEDDARSTASSSCVSTADSASSTAAAAALESEDVRRRGRRGRRAPRAEAVLEGWAKRMGLSSGAARRRAAAAGAALRAVGRGVAASRVPIRVAMAAALWSEVASSSSRRRRRPGAGQAALLLRLEASAHVPARLLLTVASWMARASTPPAAEEGWAECS

SEQ ID NO: 37 - DNA, 1094 bp, Oryza sativa
  G4298
gcacgaggcctcgtgccgaattcgggacggcgccagcgtctcgctcccaagccagacctcccccctcgccgtccgcgcgcgcgcccgcggtttcccccgctcgccgccggtttcccccgctcgccgccggtttccccgaagcgcgccgcgcccgcgcctgcgcccgccggtcgccatcgccatctcgccctcgcgcggagactggtgtccctgttttgctctgtagtataaagccacgcaaacccccgccaggtgttcgaccgagtgacacaagagtccagcctcttgcaacctgtaatggaggtcggcaacggcaagtgcggcggtggtggcgccgggtgcgagctgtgcgggggcgtggccgcggtgcactgcgccgctgactccgcgtttctttgcttggtatgtgacgacaaggtgcacggcgccaacttcctcgcgtccaggcacccccgccgccggtggggcgttgagctggtggatgatggggggcgcgcccggcgccgccccccgcccccggggggggctgggccgagtgctcctgatccgccgccgccgccggccaccgcacgacgaatcttccggccgcctgagatagaaagtactaaaaatgcgaaacttgtgggcaatgattgtttgtttgcttcctccctaattaattaaattaatctcaaattcttaatcaccatcaaggacccaaaaatcttgtggtttaggaaggcctctcttgtggttaacatcaaatcacaagtctaaatccaatggatgggactctaatttttctgtgtagtattagtataccatgatgatagtacatttgatttgttattaattggttattaattaaaggtgatttgatcaactagactttatgtggtcaaaaatgtctccctgtattgtatgagtgaccactaccactcgatatttttttccttccatcttggctgagtcctgtcttgtgtttgtttattggtatctcaatgtactgggcttaccacttgtatggacagtattgttacactaacacagtgtgtaccccccagtcgtgttagcttgaatgggaagaccatgatcaaaaaaaaaaaaaaaaaaaa

SEQ ID NO: 38 - protein, 121 aa, Oryza sativa
  G4298 polypeptide
MEVGNGKCGGGGAGCELCGGVAAVHCAADSAFLCLVCDDKVHGANFLASRHPRRRWGVELVDDGGRARRRPPPPGGAGPSAPDPPPPPATARRIFRPPEIESTKNAKLVGNDCLFASSLIN

SEQ ID NO: 39 - DNA, 750 bp, Populus trichocarpa
  4009
atggctgttaaggtctgcgagctttgcaaaggagaagctggtgtctactgcgattcagatgctgcgtatctttgttttgactgtgattctaacgtccataatgctaacttccttgttgctcgccatattcgccgtgtaatctgctccggttgcggttctatcacaggaaatccgttctccggcgacaccccatctcttagccgtgtcacctgttcctcttgctcgccaggaaacaaagaactggactccatctcctgctcctcctctagtactttatcctctgcttgcatttcaagcaccgaaacgacgcgctttgagaacacaagaaaaggagtcaagaccacgtcatcttccagctcggtgaggaatattccgggtagatccttgagggataggttgaagaggtcgaggaatctgaggtcagagggtgttttcgtgaattggtgcaaaaggctggggctcaatggtagtttggtggtacagagagccactcgggcgatggcgctgtgttttgggagattggctttgccgttcagagtgagcttagcggcgtcgttttggttcgggctcaggttatgtggggacaagtcggttacgacgtgggagaatctgaggagattagaggaggtatctggggttcccaataagctgatcgttaccgttgaaatgaagatagaacaggcgttgcgaagcaagagactgcagctgcagaaagaaatggaagaagggtgggctgagtgctctgtgtga

SEQ ID NO: 40 - protein, 249 aa, Populus trichocarpa
  G4009 polypeptide
MAVKVCELCKGEAGVYCDSDAAYLCFDCDSNVHNANFLVARHIRRVICSGCGSITGNPFSGDTPSLSRVTCSSCSPGNKELDSISCSSSSTLSSACISSTETTRFENTRKGVKTTSSSSSVRNIPGRSLRDRLKRSRNLRSEGVFVNWCKRLGLNGSLVVQRATRAMALCFGRLALPFRVSLAASFWFGLRLCGDKSVTTWENLRRLEEVSGVPNKLIVTVEMKIEQALRSKRLQLQKEMEEGWAECSV

SEQ ID NO: 41 - DNA, 1662 bp, Solanum lycopersicum
  G4299
ttattaaataataacaaactagtcaaatattacatctaccatgtaatacagtataatataaatacaatatgaatcaatggataacaaatgatccaaatgtaaatctaaatgaagataaaagagtgaatttcgcactttttatatatagagtggttaacttttgagtccacactccacaatatggtaaatgcatttatggttaatacaaagtccacaaccacaacacttggctttccttcaatctctcctttctttcctttactcaataatattactggacactcctcactttttcttttaaaccacatatataaattcaatcaataatacacttcacaaatcattctaaagtctaaattctcattacgtagcactctttgctatctcaccttactcattcctcttcctcctatatcttttctctccgccccattttcactatcacaaatcaaagcttccaaaatttagaaattgtatacaaaaatggaacttctgtcctctaaactctgtgagctttgcaatgatcaagctgctctgttttgtccatctgattcagcttttctctgttttcactgtgatgctaaagttcatcaggctaatttccttgttgctcgccaccttcgtcttactctttgctctcactgtaactcccttacgaaaaaacgtttttccccttgttcaccgccgcctcctgctctttgtccttcctgttcccggaattcgtctggtgattccgatctccgttctgtttcaacgacgtcgtcgtcgtcttcgtcgacttgtgtttccagcacgcagtccagtgctattactcaaaaaattaacataatctcttcaaatcgaaagcaatttccggacagcgactctaacggtgaagtcaattctggcagatgtaatttagtacgatccagaagtgtgaaattgcgagatccaagagcggcgacttgtgtgttcatgcattggtgcacaaagcttcaaatgaaccgcgaggaacgtgtggtgcaaacggcttgtagtgtgttgggtatttgttttagtcggtttaggggtctgcctctacgggttgccctggcggcctgtttttggtttggtttgaaaactaccgaagacaaatcaaagacgtcgcaatctttgaagaaattagaggagatctcgggtgtgccggcgaagataatattagcaacagaattaaagcttcgaaaaataatgaaaaccaaccacggccaacctcaagcaatggaagaaagctgggctgaatcctcgccctaattttctttgtttttggagaatattcccacacctcttttgattttcattttctatttttctatcttctaaatttgtgaaaaacattagaaaaatggaaaagtttgaactggaaaatccattttaccacagtattttccttttgtttttcgttttttctacatttttatcaagctgttgaaaccataaagtccgtgtcggaccaccggaaaaaatgaaaaaaaaattggaggaagaatcttctcaaaggacaaactaaaagttagacccacactatataatacatgggttcaaattcaacaaaaaataatccagggttggccccccactattaataaacttggtcaaaaattaagttttttaaaatctggggtattcacaccaaatttttatata

SEQ ID NO: 42 - protein, 261 aa, Solanum lycopersicum
  G4299 polypeptide
MELLSSKLCELCNDQAALFCPSDSAFLCFHCDAKVHQANFLVARHLRLTLCSHCNSLTKKRFSPCSPPPPALCPSCSRNSSGDSDLRSVSTTSSSSSSTCVSSTQSSAITQKINIISSNRKQFPDSDSNGEVNSGRCNLVRSRSVKLRDPRAATCVFMHWCTKLQMNREERVVQTACSVLGICFSRFRGLPLRVALAACFWFGLKTTEDKSKTSQSLKKLEEISGVPAKIILATELKLRKIMKTNHGQPQAMEESWAESSP

SEQ ID NO: 43 - DNA, 709 bp, Zea mays
  G4000
gacgtcgggaatgggcgctgctcgtgactccgcggcggcgggccagaagcacggcaccggcacgcggtgcgagctctgcgggggcgcggcggccgtgcactgcgccgcggactcggcgttcctctgcctgcgctgcgacgccaaggtgcacggcgccaacttcctggcgtccaggcacgtgaggcggcgcctggtgccgcgccgggccgccgaccccgaggcgtcgtcggccgcgtccagcggctcctcctgcgtgtccacggccgactccgcggagtcggccgccacggcaccggctccgtgcccttcgaggacggcggggaggagggctccggctcgtgcgcggcggccgcgcgcggaggcggtcctggaggggtgggccaagcggatggggttcgcggcggggccggcgcgccggcgcgccgcggcggcggccgccgcgctccgggcgctcggccggggcgtggccgctgcccgcgtgccgctccgcgtcgggatggccggcgcgctctggtcggaggtcgccgccgggtgccgaggcaatggaggggaggaggcctcgctgctccagcggctggaggccgccgcgcacgtgccggcgcggctggtgctgaccgccgcgtcgtggatggcgcgccggccggacgcccggcaggaggaccacgaggagggatgggccgagtgctcctgagttcctgatccagacggg

SEQ ID NO: 44 - protein, 225 aa, Zea mays
  G4000 polypeptide
GAARDSAAAGQKHGTGTRCELCGGAAAVHCAADSAFLCLRCDAKVHGANFLASRHVRRRLVPRRAADPEASSAASSGSSCVSTADSAESAATAPAPCPSRTAGRRAPARARRPRAEAVLEGWAKRMGFAAGPARRRAAAAAAALRALGRGVAAARVPLRVGMAGALWSEVAAGCRGNGGEEASLLQRLEAAAHVPARLVLTAASWMARRPDARQEDHEEGWAECS

SEQ ID NO: 45 - DNA, 893 bp, Zea mays
  G4297
cggacgcgtgggcggacgcgtgggcggacgcgtgggcctggagggtgcaagggagggaggcggtcggactagttctagggcggtcgaatccgccagcgcatccgctgagcaccgccagccccgcacgcggaggtcggagggctacgctccggagtccgaggggaaggcagaggaggcaagcaggcaggatgggtgccgctggtgacgccgcggcagcgggcacgcggtgcgagctctgcgggggcgcggcggccgtgcactgcgccgcggactcggcgttcctctgcccgcgctgcgacgccaaggtgcacggcgccaacttcctggcgtccaggcacgtgaggcgccgcctgccgcgcgggggcgccgactccggggcgtccgcgtccagcggctcctgcctgtccacggccgactccgtgcagtcgagggcggcgccgccgccagggagaggcagagggaggagggcgccgccgcgcgcggaggcggtgctggaggggtgggccaggaggaagggggtcgcggcggggcccgcgtgccgtcgtcgcgtcccgctccgcgtcgcgatggccgccgcgcgctggtcggaggtcagcgccggcggtggagcggaggctgcggtgctcgcagttgcggcgtggtggatgacgcgcgcggcgagagcgagacccccggcggcgggcgctccggacctggaggagggatgggccgagtgctctcctgaattcgtggtccggcagggcccacatccgtctgcaacaacatgtgggcgacgttagtttgtccttttcctccctaattattttagtaattaacgagatcgatcgtgtggtggtggtgtcgttggcttcctctcgtcgtccgattaacaaaagccggttcgatttgattac

SEQ ID NO: 46 - protein, 196 aa, Zea mays
  G4297 polypeptide
MGAAGDAAAAGTRCELCGGAAAVHCAADSAFLCPRCDAKVHGANFLASRHVRRRLPRGGADSGASASSGSCLSTADSVQSRAAPPPGRGRGRRAPPRAEAVLEGWARRKGVAAGPACRRRVPLRVAMAAARWSEVSAGGGAEAAVLAVAAWWMTRAARARPPAAGAPDLEEGWAECSPEFVVRQGPHPSATTCGRR

SEQ ID NO: 47 - DNA, 531 bp, Oryza sativa
  G5158
atgacgattaaaaggaaggacgacgggcaggtcgtgaagcaatcagtcaaagcggttggcgggggacttctagaaagggtggatagcgacgacgaggagatagtagggagggtgccggagttcgggctggcgctgccggggacgtcgacgtcgggcagaggtagtgttcgggttgcaggtgacgcggcggcgacggcggccgggacgtcgtcgtcgtcgcccgcggcgcaggccggcgtcgccggcagcagcagcagcgggcgccgccgcggacgcagccccgccgacaaggagcaccggcgcctcaaaagattgctgaggaaccgggtgtcagcgcagcaggctcgggagaggaagaaggcgtacatgagtgagctggaggcgagggtgaaggacctggagaggagcaactcagagctggaggagaggctctctaccctgcaaaacgagaaccagatgcttaggcaggtgctgaagaacacaacagcaaacagaagagggccagacagcagtgccggcggagacagctag

SEQ ID NO: 48 - protein, 176 aa, Oryza sativa
  G5158 polypeptide
MTIKRKDDGQVVKQSVKAVGGGLLERVDSDDEEIVGRVPEFGLALPGTSTSGRGSVRVAGDAAATAAGTSSSSPAAQAGVAGSSSSGRRRGRSPADKEHRRLKRLLRNRVSAQQARERKKAYMSELEARVKDLERSNSELEERLSTLQNENQMLRQVLKNTTANRRGPDSSAGGDS

SEQ ID NO: 49 - DNA, 753 bp, Oryza sativa
  G5159
atgaaggtgcagtgcgacgtgtgcgcggccgaggccgcctcggtcttctgctgcgccgacgaggccgcgctgtgcgacgcgtgcgaccgccgcgtccacagcgcgaacaagctcgccgggaagcaccgccgattctccctcctccaaccgttggcgtcgtcgtcgtccgcccagaagccaccgctctgcgacatctgtcaggagaagagggggttcttgttctgcaaggaggacagggcgatcctgtgccgggagtgcgacgtcacggtgcacaccacgagcgagctgacgaggcggcacggccggttcctcctcaccggcgtgcgcctctcgtcggcgccgatggactcccccgcgccgtcggaggaagaggaggaggaagcaggggaggactacagctgcagccccagcagcgtcgccggcaccgccgcggggagcgcgagcgacgggagcagcatctccgagtacctcaccaagacgctgcccggttggcacgtcgaggacttcctcgtcgacgaggccaccgccggcttctcctcctcagacgggctatttcagggtgggctgctggctcagatcggtggggtgccggacggttacgcggcgtgggccggccgggagcagctgcacagtggcgtcgctgtcgccgccgacgagcgggccagccgcgagcggtgggtgccgcagatgaacgcggagtggggcgccggcagcaagcgacccagggcgtcgcctccctgcttgtactggtga

SEQ ID NO: 50 - protein, 250 aa, Oryza sativa
  G5159 polypeptide
MKVQCDVCAAEAASVFCCADEAALCDACDRRVHSANKLAGKHRRFSLLQPLASSSSAQKPPLCDICQEKRGFLFCKEDRAILCRECDVTVHTTSELTRRHGRFLLTGVRLSSAPMDSPAPSEEEEEEAGEDYSCSPSSVAGTAAGSASDGSSISEYLTKTLPGWHVEDFLVDEATAGFSSSDGLFQGGLLAQIGGVPDGYAAWAGREQLHSGVAVAADERASRERWVPQMNAEWGAGSKRPRASPPCLYW

SEQ ID NO: 51 - protein, 13 aa, Arabidopsis thaliana
  G557 V-P-E/D-phi-G domain
ESDEEIRRVPEFG

SEQ ID NO: 52 - protein, 80 aa, Arabidopsis thaliana
  G557 bZIP domain
RKRGRTPAEKENKRLKRLLRNRVSAQQARERKKAYLSELENRVKDLENKNSELEERLSTLQNENQMLRHILKNTTGNKRG

SEQ ID NO: 53 - protein, 13 aa, Arabidopsis thaliana
  G1809 V-P-E/D-phi-G domain
ESDEELLMVPDME

SEQ ID NO: 54 - protein, 80 aa, Arabidopsis thaliana
  G1809 bZIP domain
RRRGRNPVDKEYRSLKRLLRNRVSAQQARERKKVYVSDLESRANELQNNNDQLEEKISTLTNENTMLRKMLINTRPKTDD

SEQ ID NO: 55 - protein, 13 aa, Glycine max
  G4631  V-P-E/D-phi-G domain
ESDEEIRRVPEIG

SEQ ID NO: 56 - protein, 80 aa, Glycine max
  G4631  bZIP domain
KKRGRSPADKESKRLKRLLRNRVSAQQARERKKAYLIDLETRVKDLEKKNSELKERLSTLQNENQMLRQILKNTTASRRG

SEQ ID NO: 57 - protein, 13 aa, Oryza sativa
  G4627  V-P-E/D-phi-G domain
ESDEEIRRVPEMG

SEQ ID NO: 58 - protein, 80 aa, Oryza sativa
  G4627  bZIP domain
RKRGRSAGDKEQNRLKRLLRNRVSAQQARERKKAYMTELEAKAKDLELRNAELEQRVSTLQNENNTLRQILKNTTAHAGK

SEQ ID NO: 59 - protein, 13 aa, Oryza sativa
  G4630  V-P-E/D-phi-G domain
ESDEEIGRVPELG

SEQ ID NO: 60 - protein, 80 aa, Oryza sativa
  G4630  bZIP domain
RRRGRSPADKEHKRLKRLLRNRVSAQQARERKKAYLNDLEVKVKDLEKKNSELEERFSTLQNENQMLRQILKNTTVSRRG

SEQ ID NO: 61 - protein, 13 aa, Zea mays
  G4632  V-P-E/D-phi-G domain
ESDEEIRRVPELG

SEQ ID NO: 62 - protein, 80 aa, Zea mays
  G4632  bZIP domain
RRRVRSPADKEHKRLKRLLRNRVSAQQARERKKAYLTDLEVKVKDLEKKNSEMEERLSTLQNENQMLRQILKNTTVSRRG

SEQ ID NO: 63 - protein, 15 aa, Oryza sativa
  G5158  V-P-E/D-phi-G domain
DSDDEEIVGRVPEFG

SEQ ID NO: 64 - protein, 80 aa, Oryza sativa
  G5158  bZIP domain
RRRGRSPADKEHRRLKRLLRNRVSAQQARERKKAYMSELEARVKDLERSNSELEERLSTLQNENQMLRQVLKNTTANRRG

SEQ ID NO: 65 - protein, 32 aa, Arabidopsis thaliana
  G1482 first ZF B-box ZF domain
KIRCDVCDKEEASVFCTADEASLCGGCDHQVH

SEQ ID NO: 66 - protein, 43 aa, Arabidopsis thaliana
  G1482 second ZF B-box domain
CDICQDKKALLFCQQDRAILCKDCDSSIHAANEHTKKHDRFLL

SEQ ID NO: 67 - protein, 32 aa, Arabidopsis thaliana
  G1888 first ZF B-box domain
KIWCAVCDKEEASVFCCADEAALCNGCDRHVH

SEQ ID NO: 68 - protein, 43 aa, Arabidopsis thaliana
  G1888 second ZF B-box domain
CDICGERRALLFCQEDRAILCRECDIPIHQANEHTKKHNRFLL

SEQ ID NO: 69 - protein, 32 aa, Oryza sativa
  G5159 first ZF B-box domain
KVQCDVCAAEAASVFCCADEAALCDACDRRVH

SEQ ID NO: 70 - protein, 43 aa, Oryza sativa
  G5159 second ZF B-box domain
CDICQEKRGFLFCKEDRAILCRECDVTVHTTSELTRRHGRFLL

SEQ ID NO: 71 - protein, 43 aa, Arabidopsis thaliana
  G1518 RING domain
LCPICMQIIKDAFLTACGHSFCYMCIITHLRNKSDCPCCSQHL

SEQ ID NO: 72 - protein, 297 aa, Arabidopsis thaliana
  G1518 WD40 domain
VSSIEFDRDDELFATAGVSRCIKVFDFSSVVNEPADMQCPIVEMSTRSKLSCLSWNKHEKNHIASSDYEGIVTVWDVTTRQSLMEYEEHEKRAWSVDFSRTEPSMLVSGSDDCKVKVWCTRQEASVINIDMKANICCVKYNPGSSNYIAVGSADHHIHYYDLRNISQPLHVFSGHKKAVSYVKFLSNNELASASTDSTLRLWDVKDNLPVRTFRGHTNEKNFVGLTVNSEYLACGSETNEVYVYHKEITRPVTSHRFGSPDMDDAEEEAGSYFISAVCWKSDSPTMLTANSQGTIKV

SEQ ID NO: 73 - protein, 43 aa, Glycine max
  G4633 RING domain
LCPICMQIIKDPFLTACGHSFCYMCIITHLRNKSDCPCCGDYL

SEQ ID NO: 74 - protein, 297 aa, Glycine max
  G4633 WD40 domain
VSSIEFDCDDDLFATAGVSRRIKVFDFSAVVNEPTDAHCPVVEMSTRSKLSCLSWNKYAKNQIASSDYEGIVTVWDVTTRKSLMEYEEHEKRAWSVDFSRTDPSMLVSGSDDCKVKIWCTNQEASVLNIDMKANICCVKYNPGSGNYIAVGSADHHIHYYDLRNISRPVHVFSGHRKAVSYVKFLSNDELASASTDSTLRLWDVKENLPVRTFKGHANEKNFVGLTVSSEYIACGSETNEVFVYHKEISRPLTCHRFGSPDMDDAEDEAGSYFISAVCWKSDRPTILTANSQGTIKV

SEQ ID NO: 75 - protein, 43 aa, Oryza sativa
  G4628 RING domain
LCPICMAVIKDAFLTACGHSFCYMCIVTHLSHKSDCPCCGNYL

SEQ ID NO: 76 - protein, 297 aa, Oryza sativa
  G4628 WD40 domain
VSSIEFDRDDELFATAGVSKRIKVFEFSTVVNEPSDVHCPVVEMATRSKLSCLSWNKYSKNVIASSDYEGIVTVWDVQTRQSVMEYEEHEKRAWSVDFSRTEPSMLVSGSDDCKVKVWCTKQEASAINIDMKANICSVKYNPGSSHYVAVGSADHHIHYFDLRNPSAPVHVFGGHKKAVSYVKFLSTNELASASTDSTLRLWDVKENCPVRTFRGHKNEKNFVGLSVNNEYIACGSETNEVFVYHKAISKPAANHRFVSSDLDDADDDPGSYFISAVCWKSDSPTMLTANSQGTIKV

SEQ ID NO: 77 - protein, 43 aa, Pisum sativum
  G4629 RING domain
LCPICMQIIKDAFLTACGHSFCYMCIITHLRNKSDCPCCGHYL

SEQ ID NO: 78 - protein, 297 aa, Pisum sativum
  G4629 WD40 domain
VSSIEFDRDDDLFATAGVSRRIKVFDFSAVVNEPTDAHCPVVEMTTRSKLSCLSWNKYAKNQIASSDYEGIVTVWTMTTRKSLMEYEEHEKRAWSVDFSRTDPSMLVSGSDDCKVKVWCTNQEASVLNIDMKANICCVKYNPGSGNYIAVGSADHHIHYYDLRNISRPVHVFTGHKKAVSYVKFLSNDELASASTDSTLRLWDVKQNLPVRTFRGHANEKNFVGLTVRSEYIACGSETNEVFVYHKEISKPLTWHRFGTLDMEDAEDEAGSYFISAVCWKSDRPTILTANSQGTIKV

SEQ ID NO: 79 - protein, 43 aa, Solanum lycopersicum
  G4635 RING domain
LCPICMQIIKDAFLTACGHSFCYMCIVTHLHNKSDCPCCSHYL

SEQ ID NO: 80 - protein, 297 aa, Solanum lycopersicum
  G4635 WD40 domain
VSSIEFDRDDELFATAGVSRRIKVFDFSSVVNEPADAHCPVVEMSTRSKLSCLSWNKYTKNHIASSDYDGIVTVWDVTTRQSVMEYEEHEKRAWSVDFSRTEPSMLVSGSDDCKVKVWCTKQEASVLNIDMKANICCVKYNPGSSVHIAVGSADHHIHYYDLRNTSQPVHIFSGHRKAVSYVKFLSNNELASASTDSTLRLWDVKDNLPVRTLRGHTNEKNFVGLSVNNEFLSCGSETNEVFVYHKAISKPVTWHRFGSPDIDEADEDAGSYFISAVCWKSDSPTMLAANSQGTIKV

SEQ ID NO: 81 - DNA, 780 bp, artificial sequence
  35S::G1988 nucleic acid construct P2499
caccatcatcattccaaaccaattctctctcacttctttctggtgatcagagagatcgactcaatggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttgagaaatattattgacatgggtcccgcattatgcaaattag

SEQ ID NO: 82 - DNA, 752 bp, artificial sequence
  35S::G4004 nucleic acid construct P26748
atgaagcccaagacttgcgagctttgtcatcaactagcttctctctattgtccctccgattccgcatttctctgcttccactgcgacgccgccgtccacgccgccaacttcctcgtagctcgccacctccgccgcctcctctgctccaaatgcaaccgtttcgccgcaattcacatctccggtgctatatcccgccacctctcctccacctgcacctcttgctccctggagattccttccgccgactccgattctctcccttcctcttctacctgcgtctccagttccgagtcttgctctacgaatcagattaaggcggagaagaagaggaggaggaggaggaggagtttctcgagttcctccgtgaccgacgacgcatctccggcggcgaagaagcggcggagaaatggcggatcggtggcggaggtgtttgagaaatggagcagagagatagggttagggttaggggtgaacggaaatcgcgtggcgtcgaacgctctgagtgtgtgcctcggaaagtggaggtcgcttccgttcagggtggctgctgcgacgtcgttttggttggggctgagattttgtggggacagaggcctcgccacgtgtcagaatctggcgaggttggaggcaatatctggagtgccagcaaagctgattctgggcgcacatgccaacctcgcacgtgtcttcacgcaccgccgcgaattgcaggaaggatggggcgagtcctagctgatgatagctataccaat

SEQ ID NO: 83 - DNA, 756 bp, artificial sequence; identical to SEQ ID NO: 31
  35S::G4005 nucleic acid construct P26749

SEQ ID NO: 84 - DNA, 709 bp, artificial sequence; identical to SEQ ID NO: 43
  35S::G4000 nucleic acid construct P27404

SEQ ID NO: 85 - DNA, 741 bp, artificial sequence
  35S::G4011 nucleic acid construct P27405
gatgggtggcgaggcggagcggtgcgcgctctgtggcgcggcggcggcggtgcactgcgaggcggacgcggcgttcctgtgcgcggcgtgcgacgccaaggtgcacggggcgaacttcctcgcgtcgcggcaccaccggaggcgggtggcggccggggcggtggtggtggtggaggtggaggaggaggaggggtatgagtccggggcgtcggcggcgtcgagcacgtcgtgcgtgtcgacggccgactccgacgtggcggcgtcggcggcggcgaggcgggggaggaggaggaggccgagggcagcggcgcggccccgcgcggaggtggttctcgaggggtggggcaagcggatgggcctcgcggcgggggcggcgcggcggcgcgccgcggcggccgggcgcgcgctccgggcgtgcggcggggacgtcgccgccgcgcgcgtcccgctccgcgtcgccatggcggccgcgctgtggtgggaggtggcggcccaccgcgtctccggcgtctccggcgccggccatgccgacgcgctgcggcggctggaggcgtgcgcgcacgtgccggcgaggctgctcacggcggtggcgtcgtcgatggcccgcgcgcgcgcaaggcggcgcgccgccgcggacaacgaggagggctgggacgagtgctcgtgttctgaagcgcccaacgccttgggtggcccacatgtcagtgacacagctcgtcagaaatgatacttatgcagagg

SEQ ID NO: 86 - DNA, 676 bp, artificial sequence
  35S::G4012 nucleic acid construct P27406
tgtaatggaggtcggcaacggcaagtgcggcggtggtggcgccgggtgcgagctgtgcgggggcgtggccgcggtgcactgcgccgctgactccgcgtttctttgcttggtatgtgacgacaaggtgcacggcgccaacttcctcgcgtccaggcaccgccgccgccggttgggggttgaggtggtggatgaggaggatgacgcccggtccacggcgtcgagctcgtgcgtgtcgacggcggactccgcgtcgtccacggcggcggcggcggcggcggtggagagcgaggacgtcaggaggagggggcggcgcgggcggcgtgccccgcgcgcggaggcggttctggaggggtgggcgaagcggatggggttgtcgtcgggcgcggcgcgcaggcgcgccgccgcggccggggcggcgctccgcgcggtgggccgtggcgtcgccgcctcccgcgtcccgatccgcgtcgcgatggccgccgcgctctggtcggaggtcgcctcctcctcctcccgtcgccgccgccgccccggcgccggacaggccgcgctgctccggcggctggaggccagcgcgcacgtgccggcgaggctgctcctgacggtggcgtcgtggatggcgcgcgcgtcgacgccgcccgccgccgaggagggctgggccgagtgctcctgatcc

SEQ ID NO: 87 - DNA, 787 bp, artificial sequence
  35S::G4299 nucleic acid construct P27428
aatggaacttctgtcctctaaactctgtgagctttgcaatgatcaagctgctctgttttgtccatctgattcagcttttctctgttttcactgtgatgctaaagttcatcaggctaatttccttgttgctcgccaccttcgtcttactctttgctctcactgtaactcccttacgaaaaaacgtttttccccttgttcaccgccgcctcctgctctttgtccttcctgttcccggaattcgtctggtgattccgatctccgttctgtttcaacgacgtcgtcgtcgtcttcgtcgacttgtgtttccagcacgcagtccagtgctattactcaaaaaattaacataatctcttcaaatcgaaagcaatttccggacagcgactctaacggtgaagtcaattctggcagatgtaatttagtacgatccagaagtgtgaaattgcgagatccaagagcggcgacttgtgtgttcatgcattggtgcacaaagcttcaaatgaaccgcgaggaacgtgtggtgcaaacggcttgtagtgtgttgggtatttgttttagtcggtttaggggtctgcctctacgggttgccctggcggcctgtttttggtttggtttgaaaactaccgaagacaaatcaaagacgtcgcaatctttgaagaaattagaggagatctcgggtgtgccggcgaagataatattagcaacagaattaaagcttcgaaaaataatgaaaaccaaccacggccaacctcaagcaatggaagaaagctgggctgaatcctcgccctaa

SEQ ID NO: 88 - protein, 84 aa, Arabidopsis thaliana
  G1518 coiled coil domain
FREALQRGCDVSIKEVDNLLTLLAERKRKMEQEEAERNMQILLDFLHCLRKQKVDELNEVQTDLQYIKEDINAVERHRIDLYRA

SEQ ID NO: 89 - protein, 84 aa, Glycine max
  G4633 coiled coil domain
FRQVLQKGSDVSIKELDTLLSLLAEKKRKMEQEEAERNMQILLDFLHCLRKQKVDELKEVQTDLHFIKEDINAVEKHRMELYRA

SEQ ID NO: 90 - protein, 84 aa, Oryza sativa
  G4628 coiled coil domain
FRYALQQGNDMAVKELDSLMTLIAEKKRHMEQQESETNMQILLVFLHCLRKQKLEELNEIQTDLQYIKEDISAVERHRLELYRT

SEQ ID NO: 91 - protein, 84 aa, Pisum sativum
  G4629 coiled coil domain
FRQAVQKGCEVTMKELDTLLLLLTEKKRKMEQEEAERNMQILLDFLHCLRKQKVDELKEVQTDLQFIKEDIGAVEKHRMDLYRA

SEQ ID NO: 92 - protein, 84 aa, Solanum lycopersicum
  G4635 coiled coil domain
FRHSLEQGSEVSIKELDALLLMLSEKKRKLEQEEAERNMQILLDFLQMLRKQKVDELNEVQHDLQYIKEDLNSVERHRIDLYRA

SEQ ID NO: 93 - protein, 13 aa, artificial sequence
  misc_feature (12)..(12): Xaa can be any naturally occurring amino acid
ESDEEIRRVPEXG

SEQ ID NO: 94 - protein, 82 aa, artificial sequence
  misc_feature (12)..(12): Xaa can be any naturally occurring amino acid
RRRGRSPADKEXKRLKRLLRNRVSAQQARERKKAYLXDLEXRVKDLEXKNSELEERLSTLQNENQMLRQILKNTTXXXXRRG

SEQ ID NO: 95 - protein, 32 aa, artificial sequence
  misc_feature (3)..(3): Xaa can be any naturally occurring amino acid
KIXCDVCDKEEASVFCCADEAALCXGCDRXVH

SEQ ID NO: 96 - protein, 43 aa, artificial sequence
  misc_feature (26)..(27): Xaa can be any naturally occurring amino acid
CDICQEKRALLFCQEDRAILCRECDXXIHXANEHTKKHXRFLL

SEQ ID NO: 97 - protein, 43 aa, artificial sequence
  misc_feature (41)..(41): Xaa can be any naturally occurring amino acid
LCPICMQIIKDAFLTACGHSFCYMCIITHLRNKSDCPCCGXYL

SEQ ID NO: 98 - protein, 84 aa, artificial sequence
  misc_feature (3)..(3): Xaa can be any naturally occurring amino acid
FRXALQXGXDVSIKELDXLLXLLAEKKRKMEQEEAERNMQILLDFLHCLRKQKVDELNEVQTDLQYIKEDINAVERHRXDLYRA

SEQ ID NO: 99 - protein, 297 aa, artificial sequence
  misc_feature (29)..(29): Xaa can be any naturally occurring amino acid
VSSIEFDRDDELFATAGVSRRIKVFDFSXVVNEPXDAHCPVVEMSTRSKLSCLSWNKYXKNXIASSDYEGIVTVWDVTTRQSLMEYEEHEKRAWSVDFSRTEPSMLVSGSDDCKVKVWCTXQEASVLNIDMKANICCVKYNPGSSNYIAVGSADHHIHYYDLRNISXPVHVFSGHKKAVSYVKFLSNNELASASTDSTLRLWDVKXNLPVRTFRGHXNEKNFVGLTVNSEYIACGSETNEVFVYHKEISKPXTXHRFGSPDMDDAEDEAGSYFISAVCWKSDSPTMLTANSQGTIKV

SEQ ID NO: 100 - DNA, 22 bp, Artificial sequence
  Synthetic oligomer primers nested within T-DNA used to isolate homozygous g1988 mutant lines, left border primer, SALK
tggttcacgtagtgggccatcg

SEQ ID NO: 101 - DNA, 30 bp, Artificial Sequence
  Forward synthetic oligomer primer on side of the predicted T-DNA insertion site used to isolate homozygous g1988 mutant lines
ggctcatgtaagtttctttgatgtgtgaac

SEQ ID NO: 102 - DNA, 28 bp, Artificial sequence
  Reverse synthetic oligomer primer on side of the predicted T-DNA insertion site used to isolate homozygous g1988 mutant lines
ctaatttgcataatgcgggacccatgtc

SEQ ID NO: 103 - DNA, 975 bp, Glycine max
  G5300 (GmHY5-2)
atggaacgaagtggcggaatggtaactgggtcgcatgaaaggaacgaacttgttagagttagacacggctctgatagtaggtctaaacccttgaagaatttgaatggtcagagttgtcaaatatgtggtgataccattggattaacggctactggtgatgtctttgtcgcttgtcatgagtgtggcttcccactttgtcattcttgttacgagtatgagctgaaacatatgagccagtcttgtccccagtgcaagactgcattcacaagtcaccaagagggtgctgaagtggagggagatgatgatgatgaagacgatgctgatgatctagataatgagatcaactatggccaaggaaacagttccaaggcggggatgctatgggaagaagatgctgacctctcttcatcttctggacatgattctcaaataccaaacccccatctagcaaacgggcaaccgatgtctggtgagtttccatgtgctacttctgatgctcaatctatgcaaactacatctataggtcaatccgaaaaggttcactcactttcatatgctgatccaaagcaaccaggtcctgagagtgatgaagagataagaagagtgccagagattggaggtgaaagtgccggaacttcggcctctcagccagatgccggttcaaatgctggtacagagcgtgttcaggggacaggggagggtcagaagaagagagggagaagcccagctgataaagaaagtaaacggctaaagaggctactgaggaaccgagtttcagctcagcaagcaagggagaggaagaaggcatacttgattgatttggaaacaagagtcaaagacttagagaagaagaactcagagctcaaagaaagactttccactttgcagaatgagaaccaaatgcttagacaaatattgaagaacacaacagcaagcaggagagggagcaataatggtaccaataatgctgagtga

SEQ ID NO: 104 - protein, 324 aa, Glycine max
  G5300 (GmHY5-2) polypeptide
MERSGGMVTGSHERNELVRVRHGSDSRSKPLKNLNGQSCQICGDTIGLTATGDVFVACHECGFPLCHSCYEYELKHMSQSCPQCKTAFTSHQEGAEVEGDDDDEDDADDLDNEINYGQGNSSKAGMLWEEDADLSSSSGHDSQIPNPHLANGQPMSGEFPCATSDAQSMQTTSIGQSEKVHSLSYADPKQPGPESDEEIRRVPEIGGESAGTSASQPDAGSNAGTERVQGTGEGQKKRGRSPADKESKRLKRLLRNRVSAQQARERKKAYLIDLETRVKDLEKKNSELKERLSTLQNENQMLRQILKNTTASRRGSNNGTNNAE

SEQ ID NO: 105 - DNA, 1215 bp, Glycine max
  G5194 (GmHY5-1, STF1a)
aagatggaacgaagtggcggaatggtaacggggtcgcatgaaaggaacgaacttgttagagttagacacggttctgacagtgggtctaaacccttgaagaatttaaatggtcagatttgtcaaatatgtggtgacaccattggattaacggctactggtgacctctttgttgcttgtcatgagtgtggcttcccactttgtcattcttgttacgagtatgagctgaaaaatgtgagccaatcttgtccccagtgcaagactacattcacaagtcgccaagagggtgctgaagtggagggagatgatgatgacgaagacgatgctgatgatctagataatgggatcaactatggccaaggaaacaattccaagtcggggatgctgtgggaagaagatgctgacctctcttcatcttctggacatgattctcatataccaaacccccatctagtaaacgggcaaccgatgtctggtgagtttccatgtgctacttctgatgctcaatctatgcaaactacatcagatcctatgggtcaatccgaaaaggttcactcacttccatatgctgatccaaagcaaccaggtcctgagagtgatgaagagataagaagagtgccggagattggaggtgaaagcgctggaacttcagcctctcggccagatgccggttcaaatgctggtacagaacgtgctcaggggacaggggacagccagaagaagagagggagaagcccagctgataaagaaagcaagcggctaaagaggctactgaggaatagagtttcggctcagcaagcaagggagaggaagaaggcatatttgattgatttggaaacaagagtcaaagacttagagaagaagaactcagagctcaaagaaagactttccactttgcagaatgaaaaccaaatgcttagacaaatattgaagaacacaacagcaagcaggcgagggagcaatagtggtaccaataatgctgtgtaaacttatagatggagtagatatagagagagagaaagaggaaagaaattaaacattcgttgatgattctttctaggtgtgcgtttggaatacaatgaagtaaaggatgaaccttaagacatgctttgtcctaaaatagtgtgatctgatgtaccattgttgatgagtaatgtaattatcatacacagttttttacagtctcattttaattaataattatcaaactacttgatt

SEQ ID NO: 106 - protein, 326 aa, Glycine max
  G5194 (GmHY5-1, STF1a) polypeptide
MERSGGMVTGSHERNELVRVRHGSDSGSKPLKNLNGQICQICGDTIGLTATGDLFVACHECGFPLCHSCYEYELKNVSQSCPQCKTTFTSRQEGAEVEGDDDDEDDADDLDNGINYGQGNNSKSGMLWEEDADLSSSSGHDSHIPNPHLVNGQPMSGEFPCATSDAQSMQTTSDPMGQSEKVHSLPYADPKQPGPESDEEIRRVPEIGGESAGTSASRPDAGSNAGTERAQGTGDSQKKRGRSPADKESKRLKRLLRNRVSAQQARERKKAYLIDLETRVKDLEKKNSELKERLSTLQNENQMLRQILKNTTASRRGSNSGTNNAV

SEQ ID NO: 107 - DNA, 576 bp, Glycine max
  G5282 GmHYH
atgtctcttccaagacccagtgagggtaaagccccttctcagctgaaagaaggagtagcacctgctgctgctgaagcctcaacctcttcttcatggaataataggctaaacacttttcctcctttatctctacacaacaagaatagcaaaattgaagacagtgatgaggatatgttcacagttccagatgtggaagccacaccaattaatgttcattctgcagtgactcttcaaaatagtaaccttaatcaacgtaatgtaacagaccctcaatttcaatctggctttcctggaaagcgccgcaggggaagaaatcctgcagataaggaacatagacgcctcaagaggttgttgcggaatagggtctctgctcaacaagcccgcgaaagaaagaaggtttatgtgaatgacttggaatcaagagctaaagagatgcaagataaaaacgctatcttagaagagcgtatctctactttaatcaatgagaacaccatgctgcggaaggttcttatgaatgcgaggccaaaaaatgatgacagcattgaacaaaagcaagaccagttaagtaagagctaa

SEQ ID NO: 108 - protein, 191 aa, Glycine max
  G5282 (GmHYH) polypeptide
MSLPRPSEGKAPSQLKEGVAPAAAEASTSSSWNNRLNTFPPLSLHNKNSKIEDSDEDMFTVPDVEATPINVHSAVTLQNSNLNQRNVTDPQFQSGFPGKRRRGRNPADKEHRRLKRLLRNRVSAQQARERKKVYVNDLESRAKEMQDKNAILEERISTLINENTMLRKVLMNARPKNDDSIEQKQDQLSKS

SEQ ID NO: 109 - DNA, 795 bp, Glycine max
  G5301 GmbZIP69
ggccccatcttgcacacacacacgtactagtactacacatttacacttttttccttcgttaaaaaatccctttgttgttgagaaggaaaaaaatagctacccttcagagcaaagaaagagagaaaaaaatgtctcttccaagacccagtgagggtaaagccccttctcagctgaaagaaggagtagcacctgctgctgctgcagcctcatcctcttcttcatggaataataggctacacactttccctcctttgtctctacacaacaagagtagcaaaattgaagacagtgatgaagatatgttcacagttcctgatgtggaaaccacaccagttagtgttcattctgcagcgactcttcaaaatagtaaccttactcaacgtaatgtgacagaccctcaatttcaaactggctttcctggaaagcgccgcaggggaagaaaccctgcagataaggaacatagacgcctcaagaggttgttgcgaaacagggtctctgcccaacaagcccgcgaaagagagaaggtttatgtgaatgacttggaatcaagagctaaagagttgcaagataaaaacgctatcttagaagaacgtatctctactttaatcaatgagaacaccatgctgcggaaggttcttatgaacgcgaggccaaaaactgatgatagcattgaacaaaagcaagaccagttaagtaagagctaacaagcaaagctagagggtgcgtcaaagtaaggcattcaagagatgcatttatgatttattttagacactagaaattgtaaatttataaata

SEQ ID NO: 110 - protein, 191 aa, Glycine max
  G5301 (GmbZIP69) polypeptide
MSLPRPSEGKAPSQLKEGVAPAAAAASSSSSWNNRLHTFPPLSLHNKSSKIEDSDEDMFTVPDVETTPVSVHSAATLQNSNLTQRNVTDPQFQTGFPGKRRRGRNPADKEHRRLKRLLRNRVSAQQAREREKVYVNDLESRAKELQDKNAILEERISTLINENTMLRKVLMNARPKTDDSIEQKQDQLSKS

SEQ ID NO: 111 - DNA, 975 bp, Glycine max
  G5302
atggaacgaagtggcggaatggtaactgggtcgcatgaaaggaacgaacttgttagagttagacacggctctgatagtaggtctaaacccttgaagaatttgaatggtcagagttgtcaaatatgtggtgataccattggattaacggctactggtgatgtctttgtcgcttgtcatgagtgtggcttcccactttgtcattcttgttacgagtatgagctgaaacatatgagccagtcttgtccccagtgcaagactgcattcacaagtcaccaagagggtgctgaagtggagggagatgatgatgatgaagacgatgctgatgatctagataatgagatcaactatggccaaggaaacagttccaaggcggggatgctatgggaagaagatgctgacctctcttcatcttctggacatgattctcaaataccaaacccccatctagcaaacgggcaaccgatgtctggtgagtttccatgtgctacttctgatgctcaatctatgcaaactacatctataggtcaatccgaaaaggttcactcactttcatatgctgatccaaagcaaccaggtcctgagagtgatgaagagataagaagagtgccagagattggaggtgaaagtgccggaacttcggcctctcagccagatgccggttcaaatgctggtacagagcgtgttcaggggacaggggagggtcagaagaagagagggagaagcccagctgataaagaaagtaaacggctaaagaggctactgaggaaccgagtttcagctcagcaagcaagggagaggaagaaggcatacttgattgatttggaaacaagagtcaaagacttagagaagaagaactcagagctcaaagaaagactttccactttgcagaatgagaaccaaatgcttagacaaatattgaagaacacaacagcaagcaggagagggagcaataatggtaccaataatgatgagtga

SEQ ID NO: 112 - protein, 324 aa, Glycine max
  G5302 polypeptide
MERSGGMVTGSHERNELVRVRHGSDSRSKPLKNLNGQSCQICGDTIGLTATGDVFVACHECGFPLCHSCYEYELKHMSQSCPQCKTAFTSHQEGAEVEGDDDDEDDADDLDNEINYGQGNSSKAGMLWEEDADLSSSSGHDSQIPNPHLANGQPMSGEFPCATSDAQSMQTTSIGQSEKVHSLSYADPKQPGPESDEEIRRVPEIGGESAGTSASQPDAGSNAGTERVQGTGEGQKKRGRSPADKESKRLKRLLRNRVSAQQARERKKAYLIDLETRVKDLEKKNSELKERLSTLQNENQMLRQILKNTTASRRGSNNGTNNDE

SEQ ID NO: 113 - protein, 12 aa, Glycine max
  G5282 (GmHYH) V-P-E/D-phi-G or G5301 domain
DSDEDMFTVPDV

SEQ ID NO: 114 - protein, 73 aa, Glycine max
  G5282 (GmHYH) bZIP domain
RRRGRNPADKEHRRLKRLLRNRVSAQQARERKKVYVNDLESRAKEMQDKNAILEERISTLINENTMLRKVLMN

SEQ ID NO: 115 - protein, 73 aa, Glycine max
  G5301 (GmHYH) bZIP domain
RRRGRNPADKEHRRLKRLLRNRVSAQQAREREKVYVNDLESRAKELQDKNAILEERISTLINENTMLRKVLMN

SEQ ID NO: 116 - DNA, 311 bp, Glycine max
  Gm_Hy5 RNAi target sequence
gggcccttttttttttttttccccccccgggaaaaagggggattttttcaaaagggtttaatttgggggaacccgagggttcggtccaggggttttaaaaaagcgaggaaatttttatagctcccctttagggggaatttgggttcggggccccccctcgagtcagctacgtaggccccccccccccccgaacaactgaagtaagaaagagagagagagagagaaagagaagtgtgtagttggtgaagtttttgagaagaatatggaacgaagtggcggaatggtaacggggtcgcatgaaaggaacgaac

SEQ ID NO: 117 - DNA, 271 bp, Glycine max
  Gm_Hyh RNAi target sequence
tctcttccaagacccagtgagggtaaagccccttctcagctgaaagaaggagtagcacctgctgctgctgaagcctcaacctcttcttcatggaataataggctaaacacttttcctcctttatctctacacaacaagaatagcaaaattgaagacagtgatgaggatatgttcacagttccagatgtggaagccacaccaattaatgttcattctgcagtgactcttcaaaatagtaaccttaatcaacgtaatgtaacagaccctcaat

SEQ ID NO: 118 - DNA, 867 bp, artificial sequence
  P21103 example base vector for the creation of RNAi constructs, poly linker and Pdk intron
ggtaccgtcgacgaggaattcggtagcccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgataatcttgcggccgctctagacaggcctcgtaccggatcc

SEQ ID NO: 119 - DNA, 1316 bp, artificial sequence
  Complete HY5 RNAi sequence, HY5 5utr plus 48bp of CDS (sense, bases 1-240), intron PDK (bases 246-1069), HY5 5utr plus 48bp of CDS (antisense, bases 1077-1316)
cagagatctgacggcggtagccagagtaatctattccttcccaaaatgtctcgcaattagattctttccaagttcttctgtaaatcccaagtcccgctcttttcctctttatccttttcaccagcttcgctactaagacaacaaatctttccctctctctctcgcctgatcgatcttcaaagagtaagaaaacaggaacaagcgactagctctttagctgcaagctctttaccatcaagcgtcgacgaggaattcggtagcccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgataatcttgcggccgcgcttgatggtaaagagcttgcagctaaagagctagtcgcttgttcctgttttcttactctttgaagatcgatcaggcgagagagagagggaaagatttgttgtcttagtagcgaagctggtgaaaaggataaagaggaaaagagcgggacttgggatttacagaagaacttggaaagaatctaattgcgagacattttgggaaggaatagattactctggctaccgccgtcagatctctg

(19) United States
(12) Patent Application Publication
(10) Pub. No.: US 2011/0173712 A1
(43) Pub. Date: Jul. 14, 2011

(54) ENHANCEMENT OF PLANT YIELD VIGOR AND STRESS TOLERANCE

(75) Inventors: Rajnish Khanna, Castro Valley, CA (US); Oliver Ratcliffe, Oakland, CA (US); T. Lynne Reuber, San Mateo, CA (US)

(73) Assignee: Mendel Biotechnology, Inc., Hayward, CA (US)

(21) Appl. No.: 12/922,834

(22) PCT Filed: Mar. 17, 2009
(86) PCT No.: PCT/US09/37439

§ 371 Date: Sep. 15, 2010

Related U.S. Application Data
(60) Provisional application No. 61/069,929, filed on Mar. 18, 2008.

(51) Int. Cl. A01H 1/02; C07H 21/00; C12N 15/82; A01H 5/00

(52) U.S. Cl. 800/260; 536/24.5; 536/23.1; 800/289; 800/290; 800/282; 800/298

(57) ABSTRACT

Altering the activity of specific regulatory proteins in plants, for example, by knocking down or knocking out HY5 clade or STH2 clade protein expression, or by modifying COP1 clade protein expression, can have beneficial effects on plant performance, including improved stress tolerance and yield.

### FIELD OF THE INVENTION

The present invention relates to plant genomics and plant improvement, increasing a plant's vigor and stress tolerance, and the yield that may be obtained from a plant.

### BACKGROUND OF THE INVENTION

### The Effects of Various Factors on Plant Yield

Yield of commercially valuable species in the natural environment is sometimes suboptimal since plants often grow under unfavorable conditions. These conditions may include an inappropriate temperature range, or a limited supply of soil nutrients, light, or water availability. More specifically, various factors that may affect yield, crop quality, appearance, or overall plant health include the following.

### Nutrient Limitation and Carbon/Nitrogen Balance (C/N) Sensing

Nitrogen (N) and phosphorus (P) are critical limiting nutrients for plants. Phosphorus is second only to nitrogen in its importance as a macronutrient for plant growth and to its impact on crop yield.

Nitrogen and carbon metabolism are tightly linked in almost every biochemical pathway in the plant. Carbon metabolites regulate genes involved in N acquisition and metabolism, and are known to affect germination and the expression of photosynthetic genes (Coruzzi et al., 2001) and hence growth. Gene regulation by C/N (carbon-nitrogen balance) status has been demonstrated for a number of N-metabolic genes (Stitt, 1999; Coruzzi et al., 2001). A plant with altered carbon/nitrogen balance (C/N) sensing may exhibit improved germination and/or growth under nitrogen-limiting conditions.

### Hyperosmotic Stresses, and Cold, and Heat

In water-limited environments, crop yield is a function of water use, water use efficiency (WUE; defined as aerial biomass yield/water use) and the harvest index [HI; the ratio of yield biomass (which in the case of a grain-crop means grain yield) to the total cumulative biomass at harvest]. WUE is a complex trait that involves water and CO₂ uptake, transport and exchange at the leaf surface (transpiration). Improved WUE has been proposed as a criterion for yield improvement under drought. Water deficit can also have adverse effects in the form of increased susceptibility to disease and pests, reduced plant growth and reproductive failure. Genes that improve WUE and tolerance to water deficit thus promote plant growth, fertility, and disease resistance.

The term “chilling sensitivity” has been used to describe many types of physiological damage produced at low, but above freezing, temperatures. Most crops of tropical origins such as soybean, rice, maize, tomato, cotton, etc. are easily damaged by chilling.

Seedlings and mature plants that are exposed to excess heat may experience heat shock, which may arise in various organs, including leaves and particularly fruit, when transpiration is insufficient to overcome heat stress. Heat also damages cellular structures, including organelles and cytoskeleton, and impairs membrane function. A transcription factor that would enhance germination in hot conditions would be useful for crops that are planted late in the season or in hot climates.

Increased tolerance to these abiotic stresses, including water deprivation brought about by low water availability, drought, salt, freezing and other hyperosmotic stresses, and cold, and heat, may improve germination, early establishment of developing seedlings, and plant development. Enhanced tolerance to these stresses could thus lead to improved germination and yield increases, and reduced yield variation in both conventional varieties and hybrid varieties.

Photoreceptors and their Impact on Plant Development

Light is essential for plant growth and development. Plants have evolved extensive mechanisms to monitor the quality, quantity, duration and direction of light. Plants perceive the informational light signal through photosensory photoreceptors; phytochromes (phy) for red (R) and Far-Red (FR) light, cryptochromes (cry) and phototropins (phot) for blue (B) light (for reviews, see Quail, 2002a; Quail 2002b and Franklin et al., 2005). The photoreceptors transmit the light signal through a cascade of transcription factors to regulate plant gene expression (Tepperman et al., 2001; Tepperman et al., 2004; and reviewed in Quail, 2000; Jiao et al., 2007).

Plants use light signals to regulate many developmental processes, including seed germination, photomorphogenesis, photoperiod (day length) perception, and flowering. Recent studies have revealed some key regulatory factors and processes involved in light signaling during seedling photomorphogenesis. Seedlings growing in the dark (etiolated seedlings) require the activity of a repressor of photomorphogenesis, CONSTITUTIVE PHOTOMORPHOGENIC 1 (COP1; SEQ ID NO: 14, encoded by SEQ ID NO: 13), which is a RING-finger type ubiquitin E3 ligase (Yi and Deng, 2005). COP1 accumulates in the nuclei in darkness and light induces its subcellular re-localization to the cytoplasm (von Arnim and Deng, 1994). COP1 acts in the dark in the nuclei to regulate degradation of multiple transcription factors such as ELONGATED HYPOCOTYL 5 (HY5; SEQ ID NO: 2 encoded by SEQ ID NO: 1) and HY5 Homolog (HYH; SEQ ID NO: 4 encoded by SEQ ID NO: 3) (Hardtke et al., 2000; Osterlund et al., 2000; Holm et al., 2002). HY5 is a basic leucine zipper (bZIP) type transcription factor; it plays a positive role in photomorphogenesis and suppresses lateral root development (Koornneef et al., 1980; Oyama et al., 1997). It has been shown that HY5 protein levels increase over 10-fold in light and that HY5 is present in a large protein complex (Hardtke et al., 2000). HY5 is phosphorylated in the dark. The unphosphorylated form of HY5 in light is more active and has higher affinity for binding its DNA targets like the G-boxes in the promoters of RBCS1a and CHS1 genes (Ang et al., 1998; Chattopadhyay et al., 1998; Hardtke et al., 2000). It has also been shown that the active, unphosphorylated form of HY5 exhibits stronger interaction with COP1 and is the preferred substrate for degradation (Hardtke et al., 2000). By this process, a small pool of phosphorylated HY5 may be maintained in the dark, which could be used for the early response during dark to light transition (Hardtke et al., 2000). HYH, the Arabidopsis homolog of HY5 functions primarily in blue-light signaling with functional overlap with HY5 (Holm et al., 2002).

### Integration of Light Signaling Pathways

Seedlings lacking HY5 function show a partially etiolated phenotype in white, red, blue, and far-red light (Koornneef et al., 1980; Ang and Deng, 1994). HY5 is thought to function downstream of all photoreceptors as a point of integration of light signaling pathways. Chromatin-immunoprecipitation experiments in combination with whole genome tiling microarrays showed that HY5 has a large number of potential DNA binding sites in promoters of known genes (Lee et al., 2007). These studies have revealed that light regulated genes are the major targets of HY5 mediated repression or activation, leading the authors to propose that HY5 functions upstream in the hierarchy of light dependent transcriptional regulation during photomorphogenesis (Jiao et al., 2007). Current knowledge of light regulated transcriptional networks suggests that transcription factors may function as homodimers or as heterodimers, pairing up with transcription factors from various families. This networking of transcription factors carries the potential of integrating signaling from different environmental cues, like light and temperature. Chromatin remodeling may act as another point of convergence from different signaling pathways. It has been shown that HISTONE ACETYLTRANSFERASE OF THE TAFII250 FAMILY (HAF2/TAF1) and GCN5, two acetyltransferases, play a positive role in light regulated transcription and HD1/HDA19, histone deacetylase, plays a negative role (Benhamed et al., 2006). Another protein, DE-ETIOLATED 1 (DET1) has been implicated in recruiting acetyltransferases (Schroeder et al., 2002). Modification of chromatin structure is likely to allow accessibility to light regulated genes. It has been suggested that the specificity for chromatin remodeling sites may be achieved by the interaction of chromatin modifying factors with transcription factors like HY5 (Jiao et al., 2007).

A B-box protein, SALT TOLERANCE HOMOLOG2 (STH2; SEQ ID NO: 24) interacts with HY5 and positively regulates light dependent transcription and seedling development (Datta et al., 2007). Seedlings lacking STH2 function are hyposensitive to blue, red and far-red light. Furthermore, like hy5 mutants, the sth2 seedlings have increased number of lateral roots and reduced anthocyanin pigment levels (Datta et al., 2007). STH2 promotes photomorphogenesis in response to multiple light wavelengths and is likely to function with HY5 in the integration of light signaling.

### Improvement of Plant Traits by Manipulating Phototransduction

The ectopic expression of a B-box zinc finger transcription factor, G1988 (SEQ ID NO: 28, encoded by SEQ ID NO: 28) has been shown to confer a number of useful traits to plants (see US patent application no. US20080010703A1). These traits include increased yield, greater height, increased secondary rooting, greater cold tolerance, greater tolerance to water deprivation, reduced stomatal conductance, altered C/N sensing, increased low nitrogen tolerance, and/or increased tolerance to hyperosmotic stress, as compared to a control plant. Orthologs of G1988 from diverse species, including eudicots and monocots, have also been shown to function in a similar manner to G1988 by conferring useful traits (see US patent application no. US20080010703A1). G1988 functions as a negative regulator in the phototransduction pathway and appears to act at the point of convergence of light signaling pathways in a manner antagonistic to HY5, SEQ ID NOs: 1 (polynucleotide) and 2 (polypeptide).

The sequences of the present invention include HY5, (SEQ ID NO: 2, and its closest Arabidopsis homolog HYH; SEQ ID NO: 3), STH2 (SEQ ID NO: 24), and COP1 (SEQ ID NO: 14). As indicated above, HY5, HYH, and STH2 proteins function positively in the phototransduction pathway, antagonistically to G1988, whereas COP1 functions to suppress phototransduction in a comparable manner to the effects of G1988. It has not previously been recognized that modifying HY5 (or HYH), STH2 or COP1 activity in plants can produce improved traits such as abiotic stress tolerance and increased yield. ZmCOP1 (Zea mays COP1) has recently been used to enhance shade avoidance response in corn (see U.S. Pat. No. 7,208,652), but it has not been recognized that overexpression of this gene could be used to enhance favorable plant properties such as abiotic stress tolerance such as water deprivation. Altering HY5 (or its homolog HYH), STH2 or COP1 expression may provide specificity in affecting phototransduction and with similar or greater yield advantage than G1988 overexpression. Furthermore, altering the expression and/or activities of these proteins at a specific phase of the photoperiod is likely to provide the desirable traits without any undesired effects that may be related to constitutive changes in their activities. It is likely that alteration of the activity of HY5, STH2, COP1, or closely related homologs of those proteins in plants will improve plant performance or yield and thus provide similar or even more beneficial traits obtained by increasing the expression of G1988 or orthologs (e.g., SEQ ID NOs: 27-46) in plants. It is likely that HY5, COP1 and STH2 will have a wide range of success over a variety of commercial crops.

We have thus identified important polynucleotide and polypeptide sequences for producing commercially valuable plants and crops as well as the methods for making them and using them. Other aspects and embodiments of the invention are described below and can be derived from the teachings of this disclosure as a whole.

### SUMMARY OF THE INVENTION

The present invention provides HY5, STH2 and COP1 clade member nucleic acid sequences (e.g., SEQ ID NOs: 1-26), as well as constructs for inhibiting or eliminating the expression of endogenous HY5 and STH2 clade member polynucleotides and polypeptides in plants, or overexpressing COP1 clade member polynucleotides and polypeptides in plants. A variety of methods for modulating the expression of HY5, STH2 and COP1 clade member nucleic acid sequences are also provided, thus conferring to a transgenic plant a number of useful and improved traits, including greater yield, greater height, increased secondary rooting, greater cold tolerance, greater tolerance to water deprivation, reduced stomatal conductance, altered C/N sensing, increased low nitrogen tolerance, and increased tolerance to hyperosmotic stress, or combinations thereof.

The invention is also directed to a nucleic acid construct comprising a recombinant nucleic acid sequence, wherein introduction of the nucleic acid construct into a plant results in a reduction or abolition of HY5 or STH2, or an enhancement of COP1, clade member gene expression or protein function.

The invention also pertains to transformed plants, and transformed seed produced by any of the transformed plants of the invention, wherein the transformed plant comprises a nucleic acid construct that suppresses (“knocks down”) or abolishes (“knocks out”) or enhances (“overexpresses”) the activity of endogenous HY5, STH2, COP1, or their closely related homologs in plants. A transformed plant of the invention may be, for example, a transgenic knockout or overexpressor plant whose genome comprises a homozygous disruption in an endogenous HY5 or STH2 clade member gene, wherein the said homozygous disruption prevents function or reduces the level of an endogenous HY5 or STH2 clade member polypeptide; or insertion of a transgene designed to produce overexpression of a COP1 clade member gene, wherein such overexpression enhances the activity or level of a COP1 clade member polypeptide. The said alterations may be constitutive or temporal by design, whereby the protein levels and/or activities are affected during a specific part of the photoperiod and expected to return to near normal levels for the rest of the photoperiod. Consequently, these changes in activity result in the transgenic knockout or overexpressing plant exhibiting increased yield, greater height, increased secondary rooting, greater cold tolerance, greater tolerance to water deprivation, reduced stomatal conductance, altered C/N sensing, increased low nitrogen tolerance, increased tolerance to hyperosmotic stress, reduced percentage of hard seed, greater average stem diameter, increased stand count, improved late season growth or vigor, increased number of pod-bearing main-stem nodes, greater late season canopy coverage, or combinations thereof, as compared to a control plant.

The presently disclosed subject matter thus also provides methods for producing a transformed plant or transformed plant seed. In some embodiments, the method comprises (a) transforming a plant cell with a nucleic acid construct comprising a polynucleotide sequence that diminishes or eliminates or increases the expression of HY5, STH2, COP1, or their homologs; (b) regenerating a plant from the transformed plant cell; and, (c) in the case of transformed seeds, isolating a transformed seed from the regenerated plant. In some embodiments, the seed may be grown into a plant that has an improved trait selected from the group consisting of enhanced yield, vigor and abiotic stress tolerance relative to a control plant (e.g., a wild-type plant of the same species, a non-transformed plant, or a plant transformed with an “empty” nucleic acid construct. The method steps may optionally comprise selfing or crossing a transgenic knockdown or knockout plant with itself or another plant, respectively, to produce a transgenic seed. In this manner, a target plant may be produced that has reduced or abolished expression of a HY5 or STH2 clade member gene, or enhanced expression of a COP1 clade member gene (where said clade includes a number of sequences phylogenetically-related to HY5, STH2 or COP1 that function in a comparable manner to those proteins and may be found in numerous plant species), wherein said transgenic knockdown or knockout or overexpressing plant exhibits the improved trait of greater yield, greater height, increased secondary rooting, greater cold tolerance, greater tolerance to water deprivation, reduced stomatal conductance, altered C/N sensing, increased low nitrogen tolerance, increased tolerance to hyperosmotic stress, reduced percentage of hard seed, greater average stem diameter, increased stand count, improved late season growth or vigor, increased number of pod-bearing main-stem nodes, greater late season canopy coverage, or combinations thereof.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

CD-ROMs Copy 1 and Copy 2, provided under 37 CFR §1.821-1.825, and Copy 3, the latter being a CRF copy of the Sequence Listing provided under 37 CFR 1.821(e) and 37 CFR §1.824, are read-only memory computer-readable compact discs. Each contains a copy of the Sequence Listing in ASCII text format. The Sequence Listing is named “MBI-0083PCT_ST25.txt”, the electronic file of the Sequence Listing contained on each of these CD-ROMs was created on Mar. 16, 2009, and is 182 kilobytes in size. The copies of the Sequence Listing on the CD-ROM discs are hereby incorporated by reference in their entirety.

FIG. 1 shows a conservative estimate of phylogenetic relationships among the orders of flowering plants (modified from Soltis et al., 1997). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al., 2001.

FIG. 2 shows a phylogenic dendrogram depicting phylogenetic relationships of higher plant taxa, including clades containing tomato and Arabidopsis; adapted from Ku et al., 2000; and Chase et al., 1993.

FIGS. 3A-3C show a multiple sequence alignment of full length HY5 and related proteins and their conserved domains (described below under DESCRIPTION OF THE SPECIFIC EMBODIMENTS).

FIGS. 4A-4B show a multiple sequence alignment of full length STH2 and related proteins and their conserved domains (described below under DESCRIPTION OF THE SPECIFIC EMBODIMENTS).

FIGS. 5A-5C show a multiple sequence alignment of full length COP1 and related proteins and their conserved domains (described below under DESCRIPTION OF THE SPECIFIC EMBODIMENTS).

FIG. 6 compares the C/N (Carbon/Nitrogen) sensitivity of two G1988 overexpressors (G1988-OX-1 and G1988-OX-2, FIGS. 6D and 6E) with their respective wild-type controls (pMEN65, which are Columbia transformed with the empty backbone vector used for G1988-OX lines; FIGS. 6A and 6B), and a hy5-1 mutant (a HY5 knockout described by Koornneef et al., 1980; FIG. 6F) with its wild-type control, Ler (FIG. 6C). All of the wild-type controls (FIGS. 6A-6C) accumulated more anthocyanin than the hy5-1 (FIG. 6F) and G1988-OX seedlings (FIGS. 6D-6E) when grown on plates under nitrogen-limiting conditions. Three biological replicates were scored visually for green color (designated as “+”) compared to their respective wild-type seedlings, and it was found that hy5-1 mutant seedlings (FIG. 6F) behaved like G1988-OEX seedlings by accumulating less anthocyanin than the wild-type controls (FIG. 6C) under all conditions tested. See Example IX below for detailed description.

FIG. 7 is a Venn diagram showing results from a microarray based transcription profiling experiment performed to compare the global gene responsivity to light between the G1988 overexpressors and the loss of function hy5 mutants. Total RNA was isolated from seedlings grown in the dark for 4 days and from seedlings exposed to 0 h, 1 h or 3 h of monochromatic red irradiation after 4 days in darkness. Global gene expression was analyzed using microarrays. All of the genes responding to the 1 h and 3 h light signal in G1988 overexpressor (black area) were compared to its control and similar analysis was done for the hy5-1 mutant (white area). In both genotypes, light responsivity was suppressed with the greatest effects after the 1 h red treatment. There was a statistically significant overlap (gray area) between downstream targets of HY5 and G1988 in response to 1 h of red light (73% of HY5 targets), indicating that differentially expressed loci from the hy5-1 mutant line are also differentially expressed in the G1988 overexpressing line. See Example VIII below for detailed description.

FIG. 8 shows hypocotyl length measurements of 7-day old seedlings grown in red light for the following genotypes: a wild-type control line (WT), a line carrying a T-DNA insertion mutation in G1988 (g1988-1), a line carrying a point mutation in HY5 (hy5-1), a line overexpressing G1988 (G1988-OEX), and a line carrying both the g1988-1 and hy5 mutations (g1988-1;hy5-1). The G1988 overexpressing line and the hy5-1 line show elongated hypocotyls in red light, while the G1988-1 line shows slightly shorter hypocotyls. The g1988-1;hy5-1 double mutant has elongated hypocotyls, indicating that hy5 is epistatic to g1988 in the g1988-1;hy5-1 double mutant. See Example XI below for detailed description.

FIG. 9 compares plants of a knockout line homozygous for a T-DNA insertion at approximately 400 bp downstream of the STH2 (G1482) start codon to controls under various stress conditions. The knockout line was more tolerant in conditions of hyperosmotic stress (10% polyethylene glycol (PEG)) as eight plants exhibited more vigorous growth than controls (FIG. 9A), eight plants exhibited more extensive root growth in low nitrogen conditions (FIG. 9B), and eight plants had more extensive root growth in phosphate-free conditions (FIG. 9C), as compared to four wild-type control plants at the right of each of the plates.

FIG. 10 shows a map of the base vector P21103.

### DETAILED DESCRIPTION OF THE INVENTION

The present invention relates to polynucleotides and polypeptides for modifying phenotypes of plants, particularly those associated with increased abiotic stress tolerance and increased yield with respect to a control plant (for example, a wild-type plant, a non-transformed plant, or a plant transformed with an “empty” nucleic acid construct lacking a polynucleotide of interest comprised within a nucleic acid construct introduced into an experimental plant). Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a nucleic acid construct, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al., 1976). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

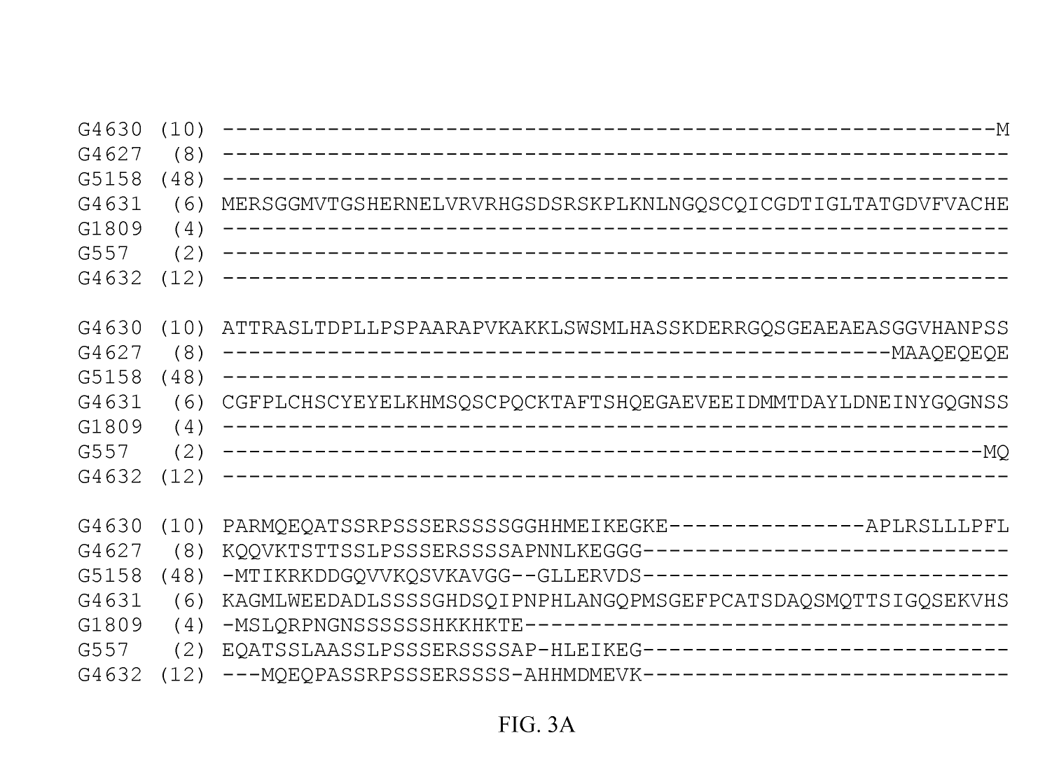

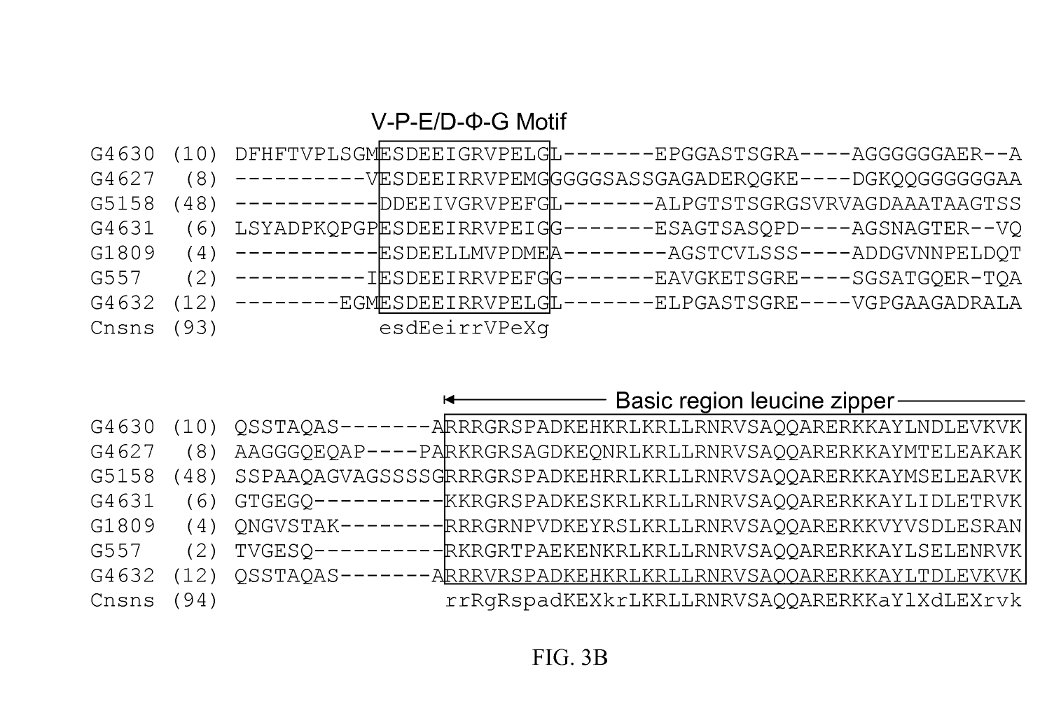

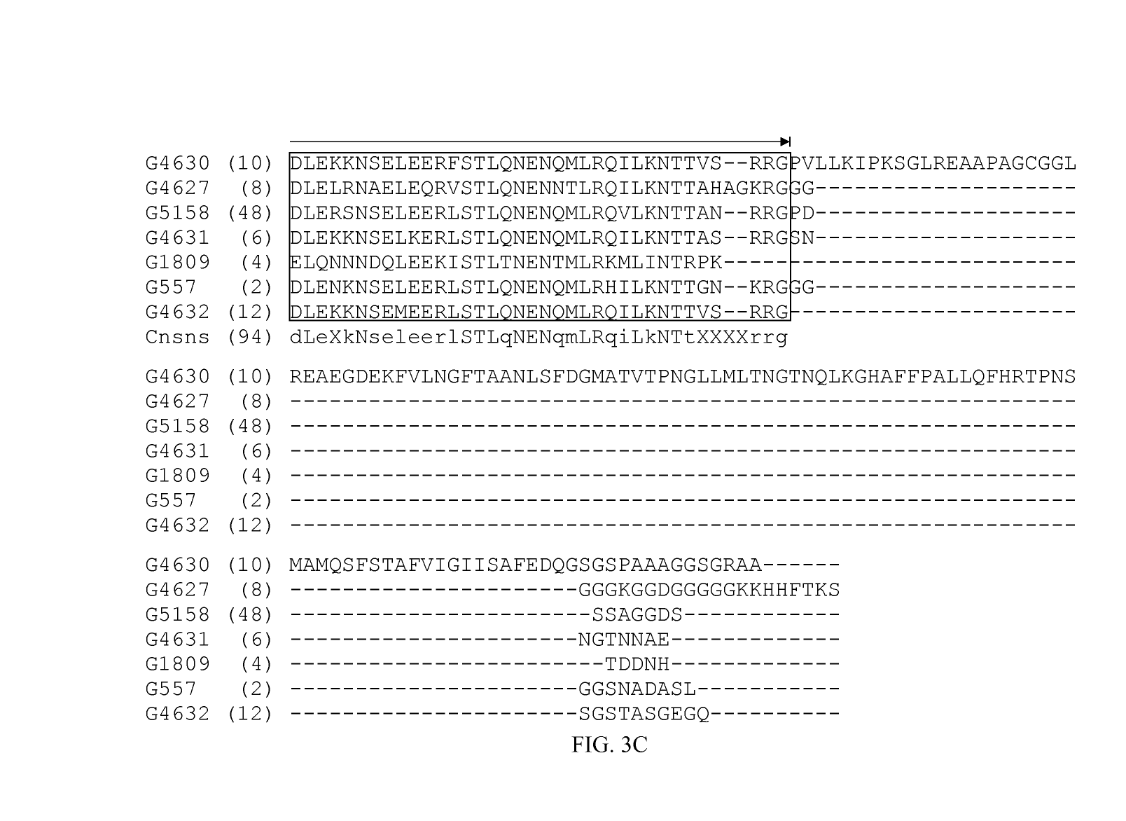

“Alignment” refers to a number of nucleotide bases or amino acid residue sequences aligned by lengthwise comparison so that components in common (i.e., nucleotide bases or amino acid residues at corresponding positions) may be visually and readily identified. The fraction or percentage of components in common is related to the homology or identity between the sequences. Alignments such as those of FIGS. 3-5 may be used to identify conserved domains and relatedness within these domains. An alignment may suitably be determined by means of computer programs known in the art, such as MACVECTOR software (1999) (Accelrys, Inc., San Diego, Calif.).

A “conserved domain” or “conserved region” as used herein refers to a region within heterogeneous polynucleotide or polypeptide sequences where there is a relatively high degree of sequence identity or homology between the distinct sequences. With respect to polynucleotides encoding presently disclosed polypeptides, a conserved domain is preferably at least nine base pairs (bp) in length. Protein sequences, including transcription factor sequences, that possess or encode for conserved domains that have a minimum percentage identity and have comparable biological activity to the present polypeptide sequences, thus being members of the same clade of transcription factor polypeptides, are encompassed by the invention. Reduced or eliminated expression of a polypeptide that comprises, for example, a conserved domain having DNA-binding, activation or nuclear localization activity, results in the transformed plant having similar improved traits as other transformed plants having reduced or eliminated expression of other members of the same clade of transcription factor polypeptides.

A fragment or domain can be referred to as outside a conserved domain, outside a consensus sequence, or outside a consensus DNA-binding site that is known to exist or that exists for a particular polypeptide class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (see, for example, Riechmann et al., 2000a, 2000b). Thus, by using alignment methods well known in the art, the conserved domains of the plant polypeptides may be determined.

The conserved domains for many of the polypeptide sequences of the invention are listed in Tables 2-4. Also, the polypeptides of Tables 2-4 have conserved domains specifically indicated by amino acid coordinate start and stop sites. A comparison of the regions of these polypeptides allows one of skill in the art (see, for example, Reeves and Nissen, 1995, to identify domains or conserved domains for any of the polypeptides listed or referred to in this disclosure.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al., 1985, Sambrook et al., 1989, and by Haymes et al., 1985, which references are incorporated herein by reference.

In general, stringency is determined by the temperature, ionic strength, and concentration of denaturing agents (e.g., formamide) used in a hybridization and washing procedure (for a more detailed description of establishing and determining stringency, see the section “Identifying Polynucleotides or Nucleic Acids by Hybridization”, below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known related polynucleotide sequences. Numerous variations are possible in the conditions and means by which nucleic acid hybridization can be performed to isolate related polynucleotide sequences having similarity to sequences known in the art and are not limited to those explicitly disclosed herein. Such an approach may be used to isolate polynucleotide sequences having various degrees of similarity with disclosed polynucleotide sequences, such as, for example, encoded transcription factors having 56% or greater identity with the conserved domain of disclosed sequences.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent polypeptide. Thus, it will be readily appreciated by those of skill in the art, that any of a variety of polynucleotide sequences is capable of encoding the polypeptides and homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the polypeptide is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the polynucleotides listed in the Sequence Listing. Exemplary fragments also include fragments that comprise a region that encodes a conserved domain of a polypeptide. Exemplary fragments also include fragments that comprise a conserved domain of a polypeptide.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

The invention also encompasses production of DNA sequences that encode polypeptides and derivatives, or fragments thereof, entirely by synthetic chemistry. After production, the synthetic sequence may be inserted into any of the many available nucleic acid constructs and cell systems using reagents well known in the art. Moreover, synthetic chemistry may be used to introduce mutations into a sequence encoding polypeptides or any fragment thereof.

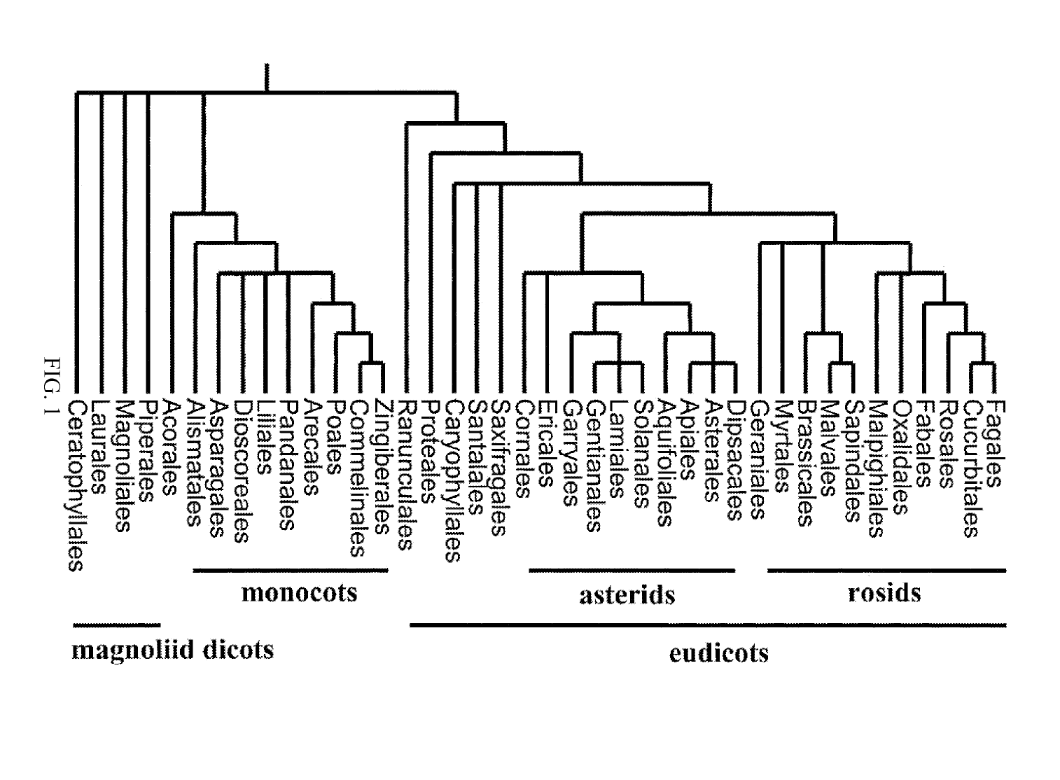

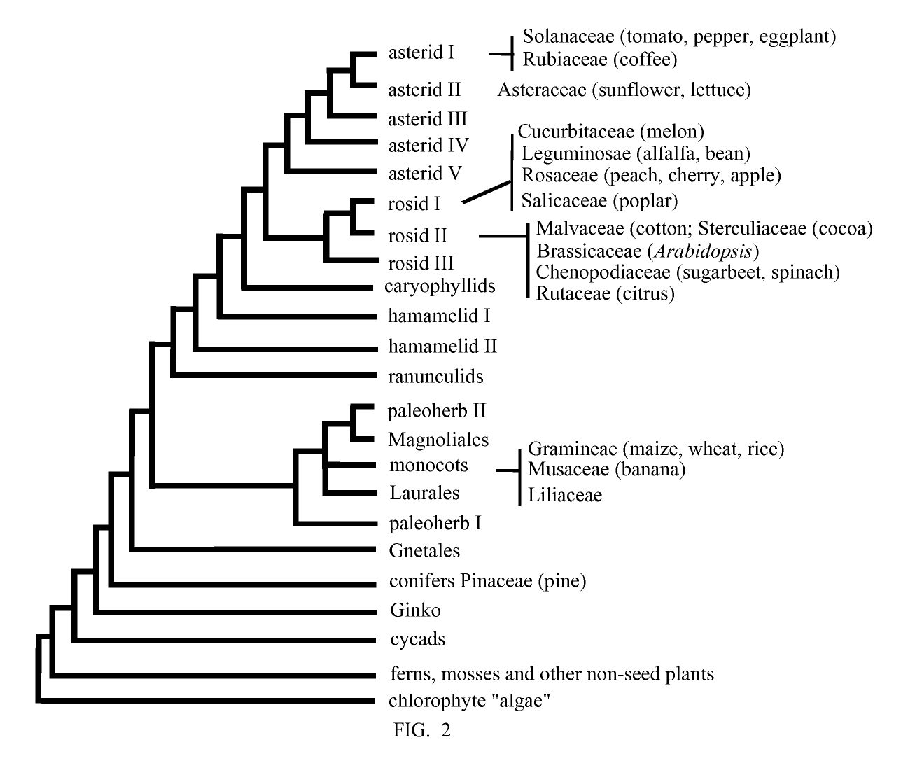

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, epidermal cells, mesophyll cells, protoplasts, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see for example, FIG. 1, adapted from Daly et al., 2001, FIG. 2, adapted from Ku et al., 2000; and see also Tudge, 2000).

A “control plant” as used in the present invention refers to a plant cell, seed, plant component, plant tissue, plant organ or whole plant used to compare against transformed, transgenic or genetically modified plant for the purpose of identifying an enhanced phenotype in the transformed, transgenic or genetically modified plant. A control plant may in some cases be a transformed or transgenic plant line that comprises an empty nucleic acid construct or marker gene, but does not contain the recombinant polynucleotide of the present invention that is expressed in the transformed, transgenic or genetically modified plant being evaluated. In general, a control plant is a plant of the same line or variety as the transformed, transgenic or genetically modified plant being tested. A suitable control plant would include a genetically unaltered or non-transgenic plant of the parental line used to generate a transformed or transgenic plant herein.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a polypeptide's expression is altered, e.g., in that it has been knocked out, overexpressed, or ectopically expressed.

“Genetically modified” refers to a plant or plant cell that has been manipulated through, for example, “Transformation” (as defined below) or traditional breeding methods involving crossing, genetic segregation, selection, and/or mutagenesis approaches to obtain a genotype exhibiting a trait modification of interest.

“Transformation” refers to the transfer of a foreign polynucleotide sequence into the genome of a host organism such as that of a plant or plant cell. Typically, the foreign genetic material has been introduced into the plant by human manipulation, but any method can be used as one of skill in the art recognizes. Examples of methods of plant transformation include Agrobacterium-mediated transformation (De Blaere et al., 1987) and biolistic methodology (U.S. Pat. No. 4,945,050 to Klein et al.).

A “transformed plant”, which may also be referred to as a “transgenic plant” or “transformant”, generally refers to a plant, a plant cell, plant tissue, seed or calli that has been through, or is derived from a plant cell that has been through, a stable or transient transformation process in which a “nucleic acid construct” that contains at least one exogenous polynucleotide sequence is introduced into the plant. The “nucleic acid construct” contains genetic material that is not found in a wild-type plant of the same species, variety or cultivar, or may contain extra copies of a native sequence under the control of its native promoter. The genetic material may include a regulatory element, a transgene (for example, a transcription factor sequence), a transgene overexpressing a protein of interest, an insertional mutagenesis event (such as by transposon or T-DNA insertional mutagenesis), an activation tagging sequence, a mutated sequence, an antisense transgene sequence, a construct containing inverted repeat sequences derived from a gene of interest to induce RNA interference, or a nucleic acid sequence designed to produce a homologous recombination event or DNA-repair based change, or a sequence modified by chimeraplasty. In some embodiments the regulatory and transcription factor sequence may be derived from the host plant, but by their incorporation into a nucleic acid construct, represent an arrangement of the polynucleotide sequences not found in a wild-type plant of the same species, variety or cultivar.

An “untransformed plant” is a plant that has not been through the transformation process.

A “stably transformed” plant, plant cell or plant tissue has generally been selected and regenerated on a selection media following transformation.

A “nucleic acid construct” may comprise a polypeptide-encoding sequence operably linked (i.e., under regulatory control of) to appropriate inducible or constitutive regulatory sequences that allow for the controlled expression of polypeptide. The expression vector or cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, to produce a recombinant plant (for example, a recombinant plant cell comprising the nucleic acid construct) as well as to progeny thereof, and to in vitro systems that mimic biochemical or cellular components or processes in a cell.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transformed or transgenic plants, however.

“Trait modification” refers to a detectable difference in a characteristic in a plant with reduced or eliminated expression, or ectopic expression, of a polynucleotide or polypeptide of the present invention relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, color, stem diameter, leaf size, leaf dimension, leaf density, internode distance, branching, root branching, number and form of inflorescences, and other macroscopic characteristics, and the individual plants are not readily distinguishable based on morphological characteristics alone.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the transcript profile of a particular polypeptide in a suspension cell is the expression levels of a set of genes in a cell knocking out or overexpressing that polypeptide compared with the expression levels of that same set of genes in a suspension cell that has normal levels of that polypeptide. The transcript profile can be presented as a list of those genes whose expression level is significantly different between the two treatments, and the difference ratios. Differences and similarities between expression levels may also be evaluated and calculated using statistical and clustering methods.

With regard to gene knockouts as used herein, the term “knockout” refers to a plant or plant cell having a disruption in at least one gene in the plant or plant cell, where the disruption results in a reduced expression (knockdown) or altered activity of the polypeptide encoded by that gene compared to a control cell. The knockout can be the result of, for example, genomic disruptions, including chemically induced gene mutations, fast neutron induced gene deletions, X-rays induced mutations, transposons, TILLING (McCallum et al., 2000), homologous recombination or DNA-repair processes, antisense constructs, sense constructs, RNA silencing constructs, RNA interference (RNAi), small interfering RNA (siRNA) or microRNA, VIGS (virus induced gene silencing) or breeding approaches to introduce naturally occurring mutant variants of a given locus. A T-DNA insertion within a gene is an example of a genotypic alteration that may abolish expression of that gene.

Ethyl methanesulfonate (EMS) is a mutagenic organic compound (C₃H₈O₃S), which causes random mutations specifically by guanine alkylation. During replication, the modified O-6-ethylguanine is paired with a thymine instead of a cytosine, converting the G:C pair to an A:T pair in subsequent cycles. This point mutation can disrupt gene function if the original codon is changed to a mis-sense, non-sense or a stop codon.

Fast neutron bombardment has been used to create libraries of plants with random genetic deletions. The library can then be screened by PCR based methods to identify individual lines carrying deletions in the gene of interest. This method can be used to obtain gene knockouts.

A “transposon” is a naturally-occurring mobile piece of DNA that can be used artificially to knock out the function of a gene into which it inserts, thus mutating the gene and more often than not rendering it non-functional. Since transposons may thus be introduced into plants and a plant with a particular mutation may be identified, this method can be used to generate plant lines that lack the function of a specific gene.

Targeting Induced Local Lesions in Genomes (“TILLING”) was first used with Arabidopsis, but has since been used to identify mutations in a specific stretch of DNA in various other plants and animals (McCallum et al., 2000). In this method, an organism's genome is mutagenized using a method well known in the art (for example, with a chemical mutagen such as ethyl methanesulfonate or a physical approach such as neuron bombardment), and then a DNA screening method is applied to identify mutations in a particular target gene. The screening method may make use of, for example, PCR-based, gel-based or sequencing-based diagnostic approaches to identify mutations.

“Homologous recombination” or “gene targeting” may be used to mutate or replace an endogenous gene with another nucleic acid segment by making use of the high degree of homology between a specific endogenous target gene and the introduced nucleic acid. This may result in a knock down or knock out of specific target gene expression, or in some cases may be used to replace an endogenous target gene with a variant engineered to have an altered level of expression or to encode a product with a modified activity. Using this approach, a vector that comprises the recombinant nucleic acid with the high degree of homology to the target DNA can be introduced into a cell or cells of an organism to introduce one or more point mutations, remove exons, or delete a large segment of the DNA target. Gene targeting can be permanent or conditional, based largely on how and when the gene of interest is normally expressed.

“RNA silencing” refers to naturally occurring and artificial processes in which expression of one or more genes is down-regulated, or suppressed completely, by the introduction of an antisense RNA molecule. Introduction of an antisense RNA molecule into plants can result in “antisense suppression” of gene expression, which involves single-stranded RNA fragments that are able to physically bind to mRNA due to the high degree of homology between the antisense RNA and the endogenous RNA, and thus block protein translation, or can cause RNA interference (defined below).

RNA interference (“RNAi”) has been used to knock down or knock out expression of numerous genes in a variety of cells and species. RNAi inhibits gene expression in a catalytic manner to cause the degradation of specific RNA molecules, thus reducing levels of the active transcript of a target RNA molecule. Small interfering RNA strands (“siRNA”), which represent one type of molecule used in RNAi methods, have complementary nucleotide stretches to a targeted RNA strand. RNAi pathway proteins cleave the mRNA target after being guided by the siRNA to the targeted mRNA. In this manner, the mRNA is rendered non-translatable. siRNAs can be exogenously introduced into cells by various transfection methods to knock down a gene of interest in a transient manner. Modified siRNAs derived from a single transcript, which are processed in vivo to produce a functional siRNAs, can be expressed by a vector that is introduced in a cell or organism of interest to produce stable suppression of protein expression.

“MicroRNAs” (miRNAs) are single-stranded RNA molecules of about 21-23 nucleotides in length that are processed from precursor molecules that are transcribed from the genome and generally function in the same manner as siRNAs. miRNAs are often derived from non-protein coding DNA, transcription of miRNAs produces short segments of non-coding RNA (the miRNA molecules) which are at least partially complementary to one or more mRNAs. The miRNAs form part of a complex with RNase activity, combine with complementary mRNAs, and thus reduce the expression level of transcripts of specific genes.

“T-DNA” (“transferred DNA”) is derived from the tumor-inducing (Ti) plasmid of Agrobacterium turnefaciens. As a generally used tool in plant molecular biology, the tumor-promoting and opine-synthesis genes are removed from the T-DNA and replaced with a polynucleotide of interest. The Agrobacterium is then used to transfer the engineered T-DNA into the plant cells, after which the T-DNA integrates into the plant genome. This technique can be used to generate transgenic plants carrying an exogenous and functional gene of interest, or can also be used to disrupt an endogenous gene of interest by the process of insertional mutagenesis.

“Virus induced gene silencing” (“VIGS”) employs viral vectors to introduce a gene or gene fragment into a plant cell to induce RNA silencing of homologous transcripts in the plant cell (Baulcombe, 1999).

“Ectopic expression or altered expression” in reference to a polynucleotide indicates that the pattern of expression in, e.g., a transformed or transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the terms “ectopic expression” or “altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression of that gene in a wild-type plant, cell or tissue, at any developmental or temporal stage. Overexpression can occur when, for example, the genes encoding one or more polypeptides are under the control of a strong promoter (e.g., the cauliflower mosaic virus 35 S transcription initiation region). Overexpression may also be achieved by placing a gene of interest under the control of an inducible or tissue specific promoter, or may be achieved through integration of transposons or engineered T-DNA molecules into regulatory regions of a target gene. Thus, overexpression may occur throughout a plant, in specific tissues of the plant, or in the presence or absence of particular environmental signals, depending on the promoter or overexpression approach used.

Overexpression may take place in plant cells normally lacking expression of polypeptides functionally equivalent or identical to the present polypeptides. Overexpression may also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors typically possess a conserved DNA binding domain. The transcription factors also comprise an amino acid subsequence that forms a transcription activation domain that regulates expression of one or more abiotic stress tolerance genes in a plant when the transcription factor binds to the regulating region.

“Yield” or “plant yield” refers to increased plant growth, increased crop growth, increased biomass, and/or increased plant product production (including grain), and is dependent to some extent on temperature, plant size, organ size, planting density, light, water and nutrient availability, and how the plant copes with various stresses, such as through temperature acclimation and water or nutrient use efficiency.

“Planting density” refers to the number of plants that can be grown per acre. For crop species, planting or population density varies from a crop to a crop, from one growing region to another, and from year to year. Using corn as an example, the average prevailing density in 2000 was in the range of 20,000-25,000 plants per acre in Missouri, USA. A desirable higher population density (which is a well-known contributing factor to yield) would be at least 22,000 plants per acre, and a more desirable higher population density would be at least 28,000 plants per acre, more preferably at least 34,000 plants per acre, and most preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al. (2003) U.S. Patent Application No. US20030101479). A desirable higher population density for each of these examples, as well as other valuable species of plants, would be at least 10% higher than the average prevailing density or yield.

### DESCRIPTION OF THE SPECIFIC EMBODIMENTS

The data presented herein represent the results obtained in experiments with polynucleotides and polypeptides that may be expressed in plants for the purpose of improving plant performance, including increasing yield, or reducing yield losses that arise from abiotic stresses.

The light signaling mechanisms described above are important for seedling establishment and throughout the life of the plant. Light and temperature signaling pathways feed into the plant circadian clock and are responsible for clock entrainment. Light signaling and the circadian clock greatly contribute towards plant growth, vigor, sustenance and yield. This invention was conceived based on our prior findings with a regulatory protein, G1988 (see US Patent Application No. US20080010703). Overexpression of G1988 in Arabidopsis causes phenotypes that suggest a negative role for G1988 in light signaling. Further experiments revealed that seedlings overexpressing G1988 are hyposensitive to multiple light wavelengths and when exposed to increasing red light fluence-rates, these overexpressors respond like photoreceptor mutants and have long hypocotyls in light. Experiments designed to distinguish between affects of G1988 overexpression on light signal transduction (phototransduction) and direct effects on the circadian clock showed that G1988 functions in the phototransduction pathway. G1988 is likely to function at the point of convergence of light signaling pathways, in a manner antagonistic to HY5 and in a comparable direction to COP1. Furthermore, we have found that increased G1988 expression can confer benefits to plants including increased tolerance to abiotic stress conditions such as osmotic stress (including water deprivation), alterations in sensitivity to C/N balance, and improved plant vigor. We have demonstrated similar effects with orthologs of G1988, showing that its activity is conserved across a wide range of plant species. Importantly, we have also shown that G1988 can be applied to increase yield in crop plants (US Patent Application No. US20080010703). Cumulatively, given the phenotypic similarities between G1988 overexpression lines and hy5 mutants, these data led to the current invention that altering the activity of HY5, STH2, COP1, or the closely related homologs of those genes (i.e., orthologs and paralogs), within crop plants will improve plant performance or yield in a similar manner as increasing G1988 activity. These proteins are likely to modulate temporally similar pathways as G1988. We predict that changing the activities of HY5, STH2, and COP1 at specific time-of-day and retaining their normal activities for the remainder of the photoperiod will provide the desirable benefits and reduce any undesired effects that may result from constant changes in their activities. The expression of such constructs could be targeted during the transition periods between the dark and light phases of the photoperiod, at the time when interactions between these proteins is expected to occur. For e.g. COP1 regulates HY5 protein expression during the night, and during the transition period between night and day; a targeted repression of HY5 activity at dawn while maintaining normal activity during the rest of the day is likely to work.

Comparison of light responsiveness of seedlings overexpressing G1988 with the light responsiveness of hy5 and g1988 mutant seedlings revealed that over 73% of the genes targeted by HY5 were also targeted by G1988 and that several classes of genes involved in light related pathways were de-repressed in the dark in g1988 mutants. These results show that a significant number of genes are common targets of G1988 and HY5, and that the native role of G1988 is likely to repress the expression of genes in the dark. It is known that STH2 interacts with HY5 and functions together with HY5 to regulate light mediated development. Our recent results have shown that G1988 is able to bind STH2 in both in vitro and protoplast based studies, which places G1988 in a potential regulatory protein complex where G1988 is likely to form functionally inactive heterodimers with STH2. Cumulatively, these data support our hypothesis that G1988 functions antagonistically to HY5 and that suppressing the activities of HY5, STH2, or related proteins will provide benefits similar to or better than the overexpression of G1988.

### Orthologs and Paralogs

Homologous sequences as described above, such as sequences that are homologous to HY5, STH2 or COP1 (SEQ ID NOs: 2, 14, or 24, respectively), can comprise orthologous or paralogous sequences (for example, SEQ ID NOs: 4, 6, 8, 10, 12, 16, 18, 20, 22, or 26). Several different methods are known by those of skill in the art for identifying and defining these functionally homologous sequences. General methods for identifying orthologs and paralogs, including phylogenetic methods, sequence similarity and hybridization methods, are described herein; an ortholog or paralog, including equivalogs, may be identified by one or more of the methods described below.

As described by Eisen, 1998, evolutionary information may be used to predict gene function. It is common for groups of genes that are homologous in sequence to have diverse, although usually related, functions. However, in many cases, the identification of homologs is not sufficient to make specific predictions because not all homologs have the same function. Thus, an initial analysis of functional relatedness based on sequence similarity alone may not provide one with a means to determine where similarity ends and functional relatedness begins. Fortunately, it is well known in the art that protein function can be classified using phylogenetic analysis of gene trees combined with the corresponding species. Functional predictions can be greatly improved by focusing on how the genes became similar in sequence (i.e., by evolutionary processes) rather than on the sequence similarity itself (Eisen, supra). In fact, many specific examples exist in which gene function has been shown to correlate well with gene phylogeny (Eisen, supra). Thus, “[t]he first step in making functional predictions is the generation of a phylogenetic tree representing the evolutionary history of the gene of interest and its homologs. Such trees are distinct from clusters and other means of characterizing sequence similarity because they are inferred by techniques that help convert patterns of similarity into evolutionary relationships . . . . After the gene tree is inferred, biologically determined functions of the various homologs are overlaid onto the tree. Finally, the structure of the tree and the relative phylogenetic positions of genes of different functions are used to trace the history of functional changes, which is then used to predict functions of [as yet] uncharacterized genes” (Eisen, supra).

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al., 1994; Higgins et al., 1996). Groups of similar genes can also be identified with pair-wise BLAST analysis (Feng and Doolittle, 1987). For example, a clade of very similar MADS domain transcription factors from Arabidopsis all share a common function in flowering time (Ratcliffe et al., 2001, and a group of very similar AP2 domain transcription factors from Arabidopsis are involved in tolerance of plants to freezing (Gilmour et al., 1998). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, 2001)

Transcription factor gene sequences are conserved across diverse eukaryotic species lines (Goodrich et al., 1993; Lin et al., 1991; Sadowski et al., 1988). Plants are no exception to this observation; diverse plant species possess transcription factors that have similar sequences and functions. Speciation, the production of new species from a parental species, gives rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al., 1994; Higgins et al., 1996) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

By using a phylogenetic analysis, one skilled in the art would recognize that the ability to predict similar functions conferred by closely-related polypeptides is predictable. This predictability has been confirmed by our own many studies in which we have found that a wide variety of polypeptides have orthologous or closely-related homologous sequences that function as does the first, closely-related reference sequence. For example, distinct transcription factors, including:

(i) AP2 family Arabidopsis G47 (found in U.S. Pat. No. 7,135,616, issued 14 Nov. 2006), a phylogenetically-related sequence from soybean, and two phylogenetically-related homologs from rice all can confer greater tolerance to drought, hyperosmotic stress, or delayed flowering as compared to control plants;

(ii) CAAT family Arabidopsis G481 (found in PCT patent publication WO2004076638), and numerous phylogenetically-related sequences from dicots and monocots can confer greater tolerance to drought-related stress as compared to control plants;

(iii) Myb-related Arabidopsis G682 (found in U.S. Pat. No. 7,193,129) and numerous phylogenetically-related sequences from dicots and monocots can confer greater tolerance to heat, drought-related stress, cold, and salt as compared to control plants;

(iv) WRKY family Arabidopsis G1274 (found in U.S. Pat. No. 7,196,245, issued 27 Mar. 2007) and numerous closely-related sequences from dicots and monocots have been shown to confer increased water deprivation tolerance, and

(v) AT-hook family soy sequence G3456 (found in US Patent Application No. US20040128712A1) and numerous phylogenetically-related sequences from dicots and monocots, increased biomass compared to control plants when these sequences are overexpressed in plants.

The polypeptides sequences belong to distinct clades of polypeptides that include members from diverse species. Knock down or knocked out approaches with canonical sequences HY5 and STH2 (SEQ ID NOs: 2 and 24) of the HY5 and STH2 clades of closely related transcription factors have been shown to confer reduced responsiveness to light, (including light-mediated gene regulation and light dependent morphological changes) or increased tolerance to one or more abiotic stresses. On the other hand, overexpression of COP1 (SEQ ID NO: 14), a member of the COP1 clade of transcription factors, was shown to inhibit light responsiveness (molecular and morphological responsiveness to light). These studies each demonstrate that evolutionarily conserved genes from diverse species are likely to function similarly (i.e., by regulating similar target sequences and controlling the same traits), and that polynucleotides from one species may be transformed into closely-related or distantly-related plant species to confer or improve traits.

The HY5, STH2 and COP1-related homologs of the invention are regulatory protein sequences that either: (a) possess a minimum percentage amino acid identity when compared to each other; or (b) are encoded by polypeptides that hybridize to another clade member nucleic acid sequence under stringent conditions; or (c) comprise conserved domains that have a minimum percentage identity and have comparable biological activity to a disclosed clade member sequence.

For example, the HY5 clade of transcription factors are examples of bZIP transcription factors that are at least about 31.9% identical to the HY5 polypeptide sequence, SEQ ID NO: 2, and each comprise V-P-E/D-φ-G and bZIP domains that are at least about 53.8% and 61.2% identical to the similar domains in SEQ ID NO: 2, respectively. The HY5 clade thus encompasses SEQ ID NOs: 2, 4, 6, 8, 10, 12 and 48, encoded by SEQ ID NOs: 1, 3, 5, 7, 9, 11, and 47, and sequences that hybridize to the latter seven nucleic acid sequences under stringent hybridization conditions.

The STH2 clade of regulator proteins are examples of Z—CO-like proteins that are at least about 35.3% identical to the STH2 polypeptide sequence, SEQ ID NO: 24, and each comprise two B-box zinc finger domains that are at least about 65.6% and 58.1% identical to the two similar respective domains in SEQ ID NO: 24. The HY5 clade thus encompasses SEQ ID NOs: 24, 26 and 50, encoded by SEQ ID NOs: 23, 25 and 49, and sequences that hybridize to the latter three nucleic acid sequences under stringent hybridization conditions.

The COP1 clade of regulator proteins are examples of RING/C3HC4 type proteins that are at least about 68.6% identical to the COP1 polypeptide sequence, SEQ ID NO: 14, and each comprise RING and WD40 domains that are at least about 81.3% and 84.8% identical to the two similar respective domains in SEQ ID NO: 14. The COP1 clade thus encompasses SEQ ID NOs: 14, 16, 18, 20 and 22, encoded by SEQ ID NOs: 13, 15, 17, 19, and 21, and sequences that hybridize to the latter five nucleic acid sequences under stringent hybridization conditions.

At the polynucleotide level, the sequences described herein in the Sequence Listing, and the sequences of the invention by virtue of a paralogous or homologous relationship with the sequences described in the Sequence Listing, will typically share at least 30%, or 40% nucleotide sequence identity, preferably at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to one or more of the listed full-length sequences, or to a region of a listed sequence excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

At the polypeptide level, the sequences described herein in the Sequence Listing and Table 2, Table 3, and Table 4, and the sequences of the invention by virtue of a paralogous, orthologous, or homologous relationship with the sequences described in the Sequence Listing or in Table 2, Table 3, or Table 4, including full-length sequences and conserved domains, will typically share at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% amino acid sequence identity or more sequence identity to one or more of the listed full-length sequences, or to a listed sequence but excluding or outside of the known consensus sequence or consensus DNA-binding site.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp (1988). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Software for performing BLAST analyses is publicly available, e.g., through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, 1990; Altschul et al., 1993). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, interne website at www.ncbi.nlm.nih.gov/).

Other techniques for alignment are described by Doolittle, 1996. Preferably, an alignment program that permits gaps in the sequence is utilized to align the sequences. The Smith-Waterman is one type of algorithm that permits gaps in sequence alignments (see Shpaer, 1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, for example, Hein, 1990) Identity between sequences can also be determined by other methods known in the art, e.g., by varying hybridization conditions (see US Patent Application No. US20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al., 1997), PFAM, and other databases which contain previously identified and annotated motifs, sequences and gene functions. Methods that search for primary sequence patterns with secondary structure gap penalties (Smith et al., 1992) as well as algorithms such as Basic Local Alignment Search Tool (BLAST; Altschul, 1990; Altschul et al., 1993), BLOCKS (Henikoff and Henikoff, 1991), Hidden Markov Models (HMM; Eddy, 1996; Sonnhammer et al., 1997), and the like, can be used to manipulate and analyze polynucleotide and polypeptide sequences encoded by polynucleotides. These databases, algorithms and other methods are well known in the art and are described in Ausubel et al., 1997, and in Meyers, 1995.

A further method for identifying or confirming that specific homologous sequences control the same function is by comparison of the transcript profile(s) obtained upon overexpression or knockout of two or more related polypeptides. Since transcript profiles are diagnostic for specific cellular states, one skilled in the art will appreciate that genes that have a highly similar transcript profile (e.g., with greater than 50% regulated transcripts in common, or with greater than 70% regulated transcripts in common, or with greater than 90% regulated transcripts in common) will have highly similar functions. Fowler and Thomashow, 2002, have shown that three paralogous AP2 family genes (CBF1, CBF2 and CBF3) are induced upon cold treatment, and each of which can condition improved freezing tolerance, and all have highly similar transcript profiles. Once a polypeptide has been shown to provide a specific function, its transcript profile becomes a diagnostic tool to determine whether paralogs or orthologs have the same function.

Furthermore, methods using manual alignment of sequences similar or homologous to one or more polynucleotide sequences or one or more polypeptides encoded by the polynucleotide sequences may be used to identify regions of similarity and conserved domains characteristic of a particular transcription factor family. Such manual methods are well-known of those of skill in the art and can include, for example, comparisons of tertiary structure between a polypeptide sequence encoded by a polynucleotide that comprises a known function and a polypeptide sequence encoded by a polynucleotide sequence that has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed polypeptides may be cloned using compositions provided by the present invention according to methods well known in the art. cDNAs can be cloned using mRNA from a plant cell or tissue that expresses one of the present sequences. Appropriate mRNA sources may be identified by interrogating Northern blots with probes designed from the present sequences, after which a library is prepared from the mRNA obtained from a positive cell or tissue. Polypeptide-encoding cDNA is then isolated using, for example, PCR, using primers designed from a presently disclosed gene sequence, or by probing with a partial or complete cDNA or with one or more sets of degenerate probes based on the disclosed sequences. The cDNA library may be used to transform plant cells. Expression of the cDNAs of interest is detected using, for example, microarrays, Northern blots, quantitative PCR, or any other technique for monitoring changes in expression. Genomic clones may be isolated using similar techniques to those.

Examples of orthologs of the Arabidopsis polypeptide sequences and their functionally similar orthologs are listed in Tables 1-3 and in the Sequence Listing as SEQ ID NOs: 1-26. In addition to the sequences in Tables 1-3 and the Sequence Listing, the invention encompasses isolated nucleotide sequences that are phylogenetically and structurally similar to sequences listed in the Sequence Listing and can function in a plant by increasing yield and/or and abiotic stress tolerance when expressed at a lower level in a plant than would be found in a control plant, a wild-type plant, or a non-transformed plant of the same species.

Since HY5 and G1988 act antagonistically in light signaling, and since a significant number of G1988-related sequences that are phylogenetically and sequentially related to each other and have been shown to enhance plant performance such as increasing yield from a plant and/or abiotic stress tolerance, the present invention predicts that HY5 and STH2, and other closely-related, phylogenetically-related, sequences which encode proteins with activity antagonistic to G1988 activity, would also perform similar functions when their expression is reduced or eliminated, and that COP1 and phylogenetically related sequences which encode proteins that act in the same direction as G1988 in light signaling would also perform similar functions when their expression is enhanced.

### Identifying Polynucleotides or Nucleic Acids by Hybridization

Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (e.g., Sambrook et al., 1989; Berger and Kimmel, 1987; and Anderson and Young 1985).

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger, 1987; and Kimmel, 1987). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al., 1989; Berger, 1987, pages 467-469; and Anderson and Young, 1985.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

Tₘ(° C.)=81.5+16.6(log [Na+])+0.41(% G+C)−0.62(% formamide)−500/L (I) DNA-DNA

Tₘ(° C.)=79.8+18.5(log [Na+])+0.58(% G+C)+0.12(% G+C)²−0.5(% formamide)−820/L (II) DNA-RNA:

Tₘ(° C.)=79.8+18.5(log [Na+])+0.58(% G+C)+0.12(% G+C)²−0.35(% formamide)−820/L (III) RNA-RNA

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young, 1985). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ-5° C. to Tₘ-20° C., moderate stringency at Tₘ-20° C. to Tₘ-35° C. and low stringency at Tₘ-35° C. to Tₘ-50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ-25° C. for DNA-DNA duplex and Tₘ-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present polypeptides include, for example:

6×SSC at 65° C.;

50% formamide, 4×SSC at 42° C.; or

0.5×SSC to 2.0×SSC, 0.1% SDS at 50° C. to 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. US20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a polypeptide known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger, 1987, pages 399-407; and Kimmel, 1987). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

### Sequence Variations

It will readily be appreciated by those of skill in the art that the instant invention includes any of a variety of polynucleotide sequences provided in the Sequence Listing or capable of encoding polypeptides that function similarly to those provided in the Sequence Listing or Tables 1, 2 or 3. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (that is, peptides having some degree of equivalent or similar biological activity) but differ in sequence from the sequence shown in the sequence listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide. For example, substitutions, deletions and insertions introduced into the sequences provided in the Sequence Listing are also envisioned. Such sequence modifications can be engineered into a sequence by site-directed mutagenesis (for example, Olson et al., Smith et al., Zhao et al., and other articles in Wu (ed.) Meth. Enzymol. (1993) vol. 217, Academic Press) or the other methods known in the art or noted herein. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 1 when it is desired to maintain the activity of the protein. Table 1 shows amino acids which can be substituted for an amino acid in a protein and which are typically regarded as conservative substitutions.

TABLE 1
Possible conservative amino acid substitutions

| Amino Acid | |
|---|---|
| Residue | Conservative substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The polypeptides provided in the Sequence Listing have a novel activity, such as, for example, regulatory activity. Although all conservative amino acid substitutions (for example, one basic amino acid substituted for another basic amino acid) in a polypeptide will not necessarily result in the polypeptide retaining its activity, it is expected that many of these conservative mutations would result in the polypeptide retaining its activity. Most mutations, conservative or non-conservative, made to a protein but outside of a conserved domain required for function and protein activity will not affect the activity of the protein to any great extent.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention. It will be recognized by one of skill in the art that a polypeptide that is associated with a particular first trait may also be associated with at least one other, unrelated and inherent second trait which was not predicted by the first trait.

### Example I

### Transcription Factor Polynucleotide and Polypeptide Sequences of the Invention: Background Information for HY5, STH2, COP1, SEQ ID NOs: 2, 24 and 14, and Related Sequences

### HY5 and Related Proteins

ELONGATED HYPOCOTYL 5 (HY5) and HY5 HOMOLOG (HYH) constitute Group H of the Arabidopsis basic/leucine zipper motif (AtbZIP) family of transcription factors, which consists of 75 distinct family members classified into different Groups based upon their common domains (Jakoby et al., 2002). HY5 and related proteins contain a structural motif (core sequence, V-P-E/D-φ-G; φ=hydrophobic residue), which is necessary for specific interaction with the WD40 repeat domain of COP1 (Holm et al., 2001). A multiple sequence alignment of full length HY5 and related proteins is shown in FIG. 3. Table 2 shows the amino acid positions of the V-P-E/D-φ-G and bZIP domains in HY5 (G557), and its clade members (G1809, G4631, G4627, G4630, G4632 and G5158) from Arabidopsis, soy, rice and maize. All of these proteins are expected to bind regulatory promoter elements like the G-box through the bZIP domain and interact with COP1 like proteins through the V-P-E/D-φ-G motif.

### STH2 and Related Proteins

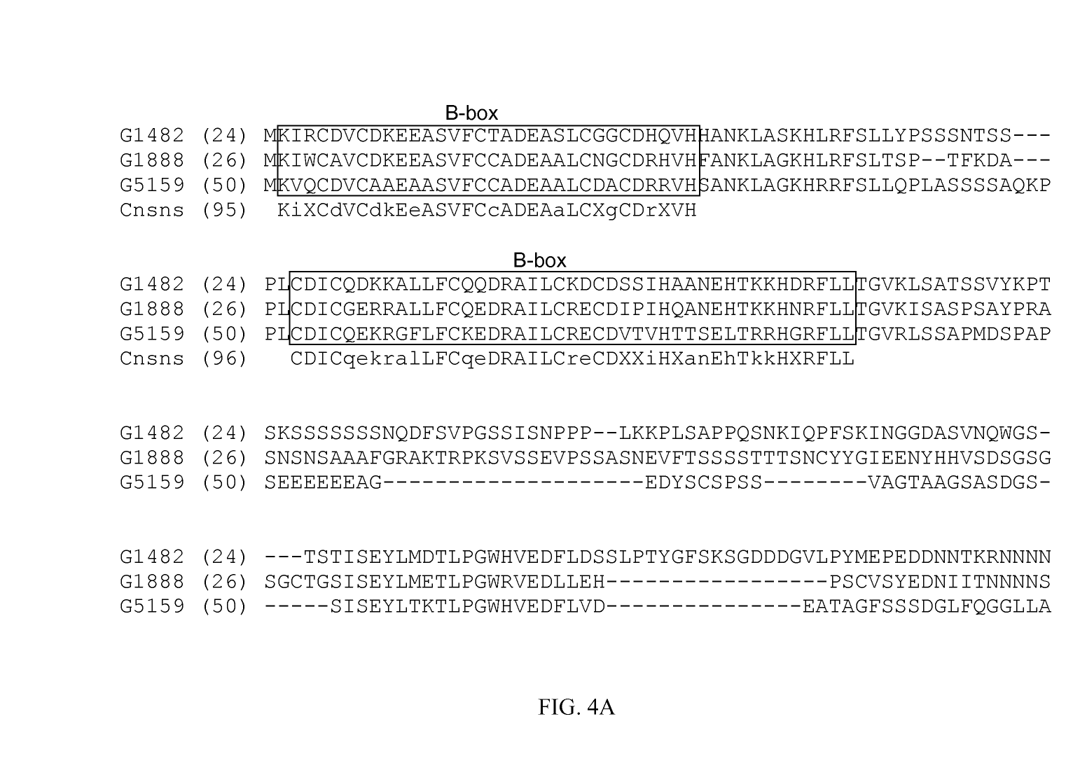

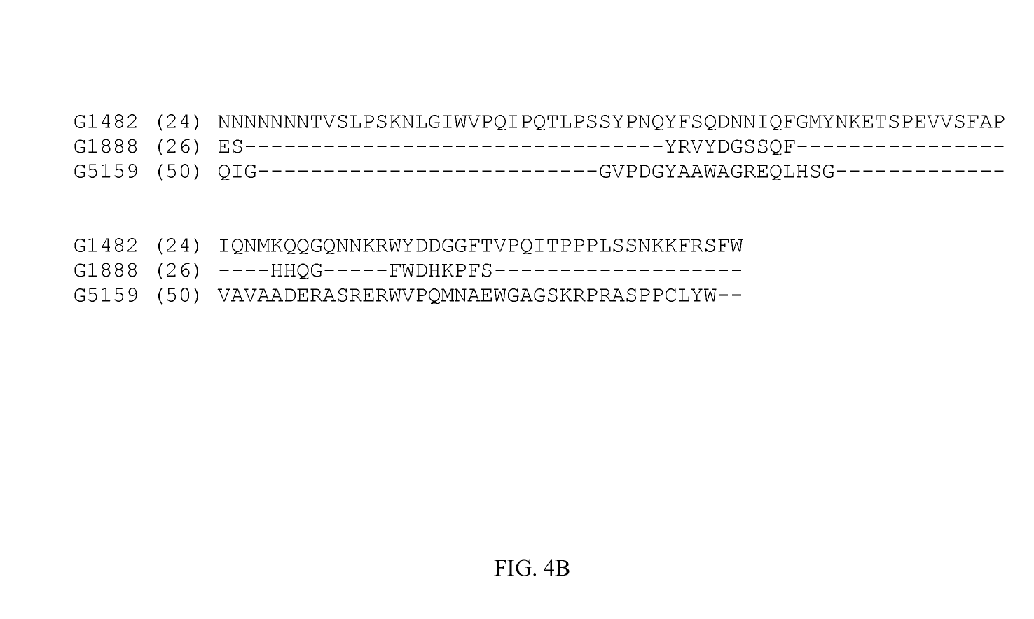

SALT TOLERANCE HOMOLOG2 (STH2) contains two B-box domains. The B-box is a Zn²⁺-binding domain and consists of conserved Cys and H is residues (Borden et al., 1995; Torok and Etkin, 2001; see Patent Application No. US20080010703A1). In Arabidopsis, 32 B-box containing proteins were initially described as “transcription factors” (Riechmann et al., 2000a), but the molecular function of B-box proteins has not yet been experimentally proven. Recent studies have shown that STH2 functions positively in photomorphogenesis and that the two B-boxes in STH2 are required for its interaction with HY5 (Datta et al., 2007). A multiple sequence alignment of full length STH2 and related proteins is shown in FIG. 4. Table 3 shows the amino acid positions of the two B-box domains in STH2 (G1482) and its clade members (G1888 and G5159) from Arabidopsis and rice. It is not yet known whether these proteins can directly bind DNA. The B-boxes are likely to be involved in protein-protein interactions.

### COP1 and Related Proteins

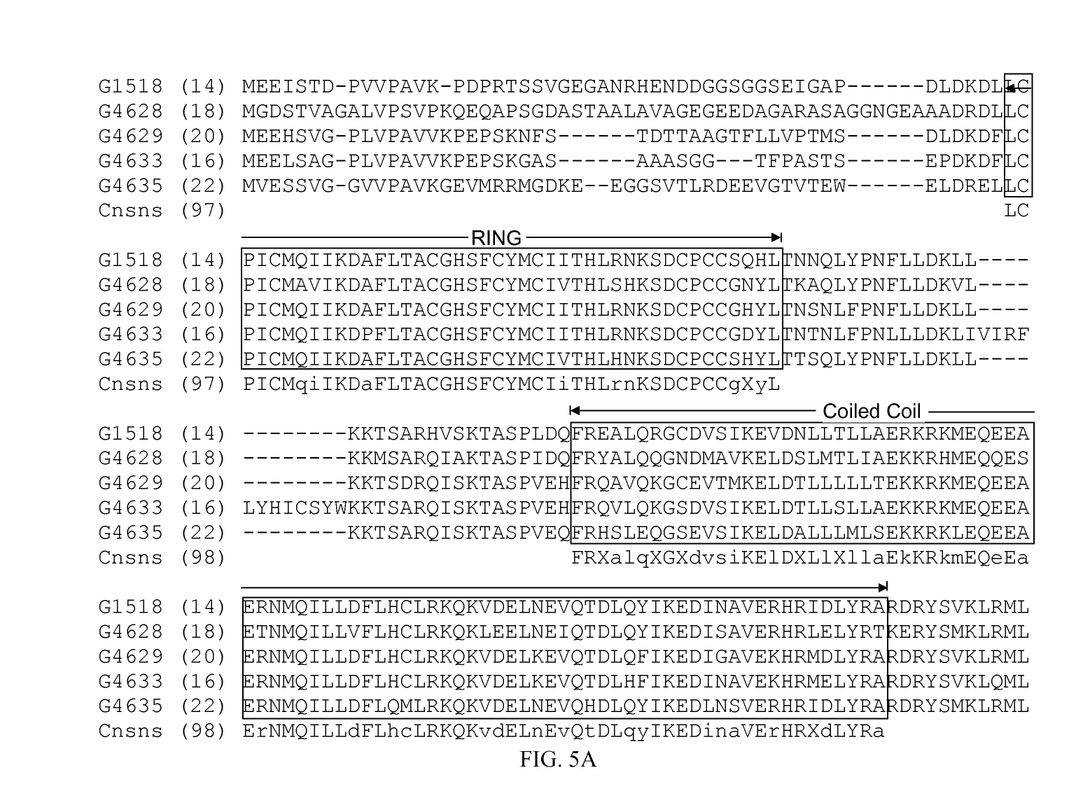

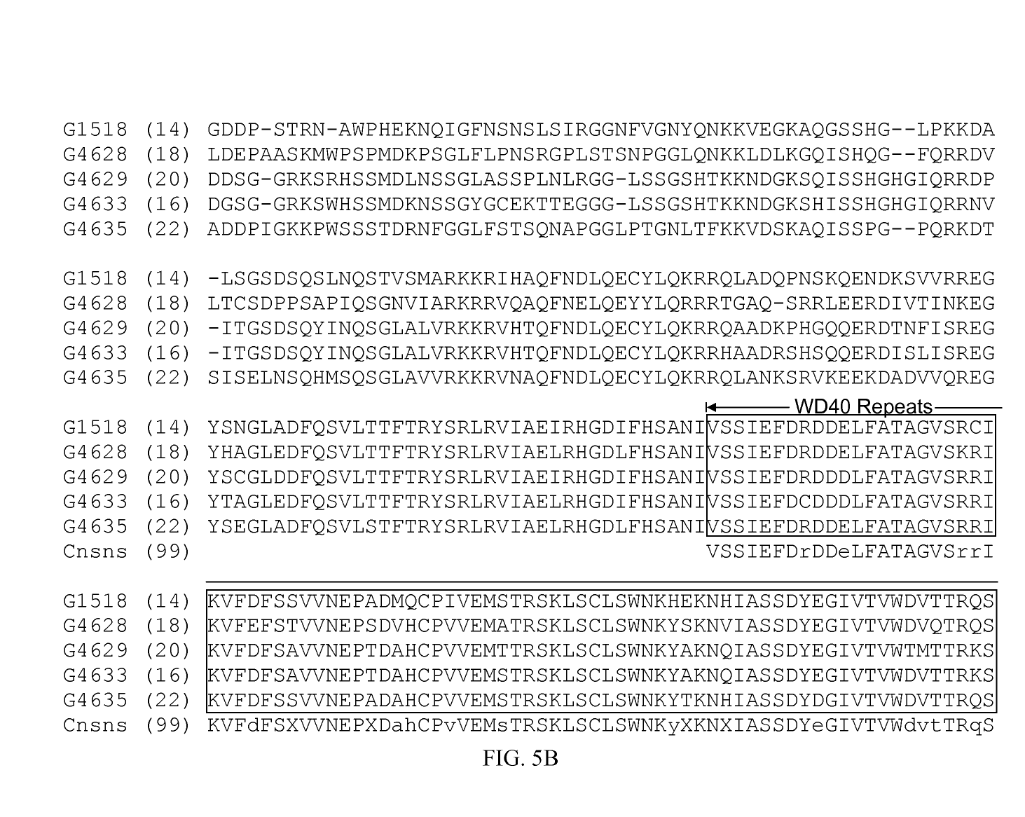

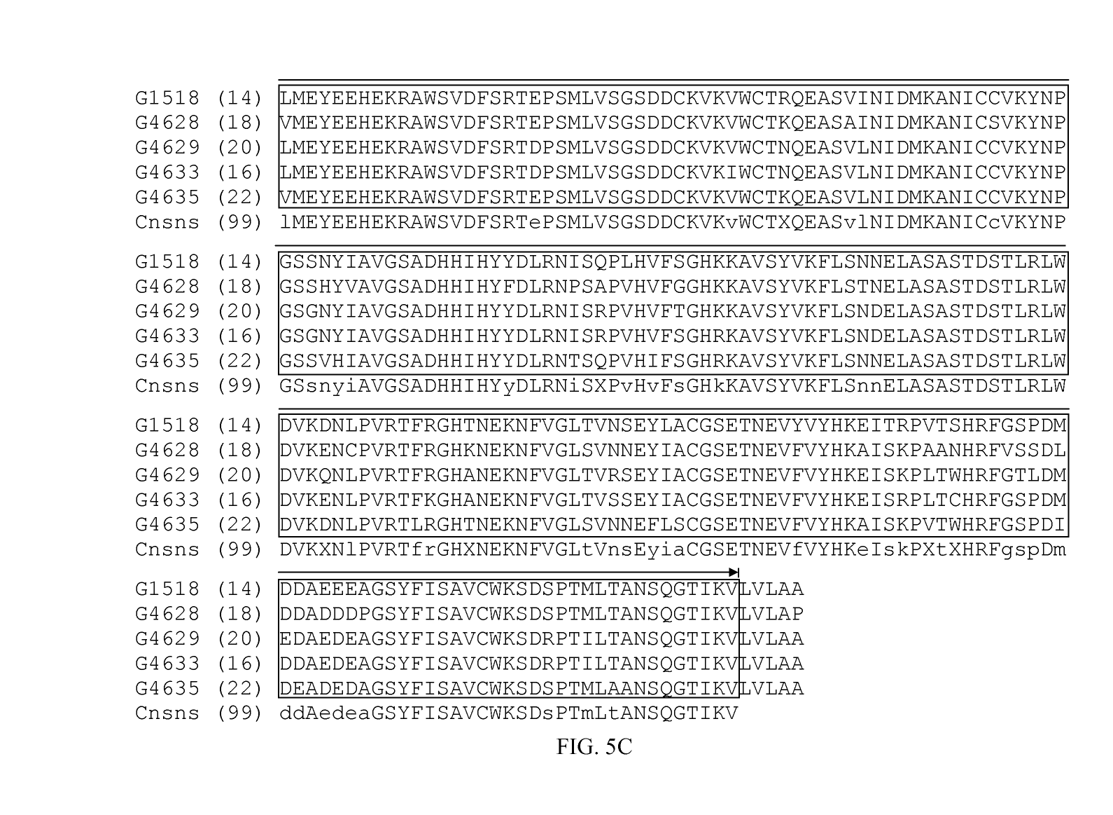

CONSTITUTIVE PHOTOMORPHOGENIC 1 (COP1) is an E3 ubiquitin ligase involved in the degradation of HY5 and HYH, as well as other transcription factors which promote photomorphogenesis (Osterlund et al., 2000; Holm et al., 2002). COP1 contains three domains; a Zn²′-ligating RING finger domain, a coiled-coil domain and seven WD-40 repeats (Deng et al., 1992; McNellis et al., 1994). A multiple sequence alignment of full length COP1 and related proteins is shown in FIG. 5. Table 4 shows the amino acid positions of the Ring finger and the WD-40 Repeats in COP1 (G1518) and its clade members (G4633, G4628, G4629 and G4635) from Arabidopsis, soy, rice, pea and tomato. COP1 and related proteins are expected to regulate light signaling pathways by directly interacting with and degrading other proteins.

Representative HY5, STH2 and COP1 clade member genes and their conserved domains are provided in Table 2-4. Species abbreviations for Tables 2-4 include At=Arabidopsis thaliana; Gm=Glycine max; Os=Oryza sativa; Ps=Pisum sativum; Sl=Solanum lycopersicum; Zm=Zea mays.

TABLE 2
Conserved domains of HY5 (G557; SEQ ID NO: 2) and closely related sequences

| | | | | Column 5 | Column 6 |
|---|---|---|---|---|---|
| | | | | SEQ ID | Percent identity of |
| | | | Column 4 | NOs: of V- | V-P-E/D-φ-G and |
| Column 1 | | Column 3 | Amino acid | P-E/D-φ-G | bZIP domains in |
| Polypeptide | Column 2 | Percent identity of | coordinates of V- | and bZIP | Column 5 to |
| SEQ ID | Species/ | polypeptide in Column | P-E/D-φ-G and | domains, | conserved domain of |
| NO: | GID No. | 1 to G557* | bZIP domain | respectively | G557** |
| 2 | At/G557 | Acc: 100.0% | V-P-E: 35-47 | 51, 52 | Acc: 100.0%, 100.0% |
| | | Blast: 100% (168/168) | bZIP: 78-157 | | |
| 4 | At/G1809 | Acc: 44.3% | V-P-E: 23-35 | 53, 54 | Acc: 53.8%, 61.3% |
| | | Blast: 49% (70/141) | bZIP: 68-147 | | |
| 6 | Gm/G4631 | Acc: 63.0% | V-P-E: 192-204 | 55, 56 | Acc: 92.3%, 83.8% |
| | | 62% (102/162) | bZIP: 234-313 | | |
| 8 | Os/G4627 | Acc: 53.9% | V-P-E: 43-55 | 57, 58 | Acc: 92.3%, 70.0% |
| | | Blast: 57% (104/180) | bZIP: 100-179 | | |
| 10 | Os/G4630 | Acc: 61.4% | V-P-E: 118-130 | 59, 60 | Acc: 84.6%, 82.5% |
| | | Blast: 61% (113/183) | bZIP: 163-242 | | |
| 12 | Zm/G4632 | Acc: 63.0% | V-P-E: 32-44 | 61, 62 | Acc: 92.3%, 81.3% |
| | | Blast: 67% (115/171) | bZIP: 79-158 | | |
| 48 | Os/G5158 | Acc: 53.2% | V-P-E: 30-42 | 63, 64 | Acc: 69.2%, 83.8% |
| | | Blast: 50% (88/173) | bZIP: 88-167 | | |
| 104 | Gm/G5300 | Acc: 63.0% | V-P-E: 194-206 | 55, 56 | Acc: 92.3%, 83.8% |
| | | Blast: 62% (102/162) | bZIP: 236-315 | | |
| 106 | Gm/G5194 | Acc: 63.6% | V-P-E: 196-208 | 55, 56 | Acc: 92.3%, 83.8% |
| | | Blast: 64% (102/157) | bZIP: 238-317 | | |
| 108 | Gm/G5282 | Acc: 35.9% | V-P-E: 53-64 | 113, 114 | Acc: 41.7%, 68.5% |
| | | Blast: 41% (67/163) | bZIP: 100-172 | | |
| 110 | Gm/G5301 | Acc: 35.9% | V-P-E: 53-64 | 113, 115 | Acc: 41.7%, 68.5% |
| | | Blast: 44% (68/153) | bZIP: 100-172 | | |
| 112 | Gm/G5302 | Acc: 63.6% | V-P-E: 194-206 | 55, 56 | Acc: 92.3%, 83.8% |
| | | Blast: 62% (103/164) | bZIP: 236-315 | | |

*First value listed was determined with Accelrys Gene v.2.5/second value listed determined by BLAST
**Values for both domains determined with Accelrys Gene v.2.5

TABLE 3
Conserved domains of STH2 (G1482; SEQ ID NO: 24) and closely related sequences

| | | | | | Column 6 |
|---|---|---|---|---|---|
| | | | | | Percent identity of |
| | | | Column 4 | Column 5 | B-box zinc finger |
| Column 1 | | Column 3 | Amino acid | SEQ ID | domain in Column |
| Polypeptide | Column 2 | Percent identity of | coordinates of B- | NOs: of B- | 5 to conserved |
| SEQ ID | Species/ | polypeptide in | box zinc finger | box ZF | domain of |
| NO: | GID No. | Column 1 to G1482* | domains | domains | G1482** |
| 24 | At/G1482 | 100.0%/100% | 2-33 and 60-102 | 65, 66 | 100%, 100% |
| 26 | At/G1888 | 51.7%/53.4% | 2-33 and 58-100 | 67, 68 | 78.1%, 74.4% |
| 50 | Os/G5159 | 40.5%/47.1% | 2-33 and 63-105 | 69, 70 | 65.6%, 58.1% |

*First value listed was determined with Accelrys Gene v.2.5/second value listed determined by BLAST
**Values for both domains determined with Accelrys Gene v.2.5

TABLE 4
Conserved domains of COP1 (G1518; SEQ ID NO: 14) and closely related sequences

| | | | | Column 5 | Column 6 |
|---|---|---|---|---|---|
| | | Column 3 | Column 4 | SEQ ID NOs: | Percent identity of |
| | | Percent | Amino acid | of RING, | RING, Coiled Coil |
| Column 1 | | identity of | coordinates of | Coiled Coil, | and WD40 domains, |
| Polypeptide | Column 2 | polypeptide | RING, Coiled | and WD40 | respectively, to |
| SEQ ID | Species/GID | in Column 1 | Coil (CC) and | domains, | conserved domain of |
| NO: | No. | to G1518* | WD40 domains | respectively | G1518** |
| 14 | At/G1518 | 100%/ | RING: 51-93 | 71, 88, 72 | 100%, 100%, 100% |
| | | 100% | CC: 126-209 | | |
| | | | WD40: 374-670 | | |
| 16 | Gm/G4633 | 75.7%/ | RING: 43-85 | 73, 89, 74 | 90.6%, 83.3%, 88.9% |
| | | 74.8% | CC: 130-213 | | |
| | | | WD40: 380-676 | | |
| 18 | Os/G4628 | 69.1%/ | RING: 59-101 | 75, 90, 76 | 81.4%, 72.6%, 84.8% |
| | | 70.1% | CC: 134-217 | | |
| | | | WD40: 384-680 | | |
| 20 | Ps/G4629 | 76.7%/ | RING: 46-88 | 77, 91, 78 | 93.0%, 81.0%, 87.5% |
| | | 76.0% | CC: 121-204 | | |
| | | | WD40: 371-667 | | |
| 22 | Sl/G4635 | 75.4%/ | RING: 50-92 | 79, 92, 80 | 90.7%, 78.6%, 89.6% |
| | | 76.4% | CC: 125-208 | | |
| | | | WD40: 376-672 | | |

*First value listed was determined with Accelrys Gene v.2.5/second value listed determined by BLAST
**Values for both domains determined with Accelrys Gene v.2.5

### Example II

### Methods for Modulation of Gene Expression in Plants

### Constructs for Gene Overexpression

A number of constructs were used to modulate the activity of sequences of the invention. For overexpression of genes, the sequence of interest was typically amplified from a genomic or cDNA library using primers specific to sequences upstream and downstream of the coding region and directly fused to the cauliflower mosaic virus 35S promoter, that drove drive its constitutive expression in transgenic plants. Alternatively, a promoter that drives tissue specific or conditional expression could be used in similar studies. Constructs used in this study are described in the table below.

TABLE 5
Expression constructs used to create plants overexpressing G1988 clade members

| Gene Identifier | | | | |
|---|---|---|---|---|
| (SEQ ID NO) | Construct | SEQ ID NO: | | |
| Species | (PID) | of PID | Promoter | Construct Design |
| G1988 (28) At | P2499 | 81 | 35S | Direct promoter-fusion |
| G4004 (30) Gm | P26748 | 82 | 35S | Direct promoter-fusion |
| G4005 (32) Gm | P26749 | 83 | 35S | Direct promoter-fusion |
| G4000 (44) Zm | P27404 | 84 | 35S | Direct promoter-fusion |
| G4011 (34) Os | P27405 | 85 | 35S | Direct promoter-fusion |
| G4012 (36) Os | P27406 | 86 | 35S | Direct promoter-fusion |
| G4299 (42) Sl | P27428 | 87 | 35S | Direct promoter-fusion |

Species abbreviations for Table 5: At—Arabidopsis thaliana; Gm—Glycine max; Os—Oryza sativa; Sl—Solanum lycopersicum; Zm—Zea mays

Identification of Plant Lines with Gene Mutations

The hy5-1 mutant (Koornneef et al., 1980) used in this study is an EMS mutant allele, which has the fourth codon (CAA) substituted for a stop codon (TAA) (Oyama et al., 1997) and lacks HY5 protein (Osterlund et al., 2000).

The G1988 mutant used in our study is a T-DNA insertion allele. A single T-DNA insertional-disruption mutant (SALK_{—}059534) was identified in the ABRC collection (Alonso et al., 2003). The site of T-DNA insertion is predicted to be 671 bp downstream of the transcriptional start site and 518 bp downstream of the ATG start codon. Synthetic oligomer primers nested within the T-DNA (Lb=TGGTTCACGTAGTGGGCCATCG (SEQ ID NO: 100); left border primer, SALK) and on either side of the predicted insertion site (F=GGCTCATGTAAGTTTCTTTGATGTGTGAAC (SEQ ID NO: 101); R═CTAATTTGCATAATGCGGGACCCATGTC (SEQ ID NO: 102)) were used to isolate homozygous g1988 mutant lines by PCR analysis. A wild type sibling (WT) lacking the T-DNA was maintained for use as a control.

### Example III

### Transformation Methods

Transformation of Arabidopsis is performed by an Agrobacterium-mediated protocol based on the method of Bechtold and Pelletier, 1998. Unless otherwise specified, all experimental work is done using the Columbia ecotype.

Plant preparation. Arabidopsis seeds are sown on mesh covered pots. The seedlings are thinned so that 6-10 evenly spaced plants remain on each pot 10 days after planting. The primary bolts are cut off a week before transformation to break apical dominance and encourage auxiliary shoots to form. Transformation is typically performed at 4-5 weeks after sowing.

Bacterial culture preparation. Agrobacterium stocks are inoculated from single colony plates or from glycerol stocks and grown with the appropriate antibiotics and grown until saturation. On the morning of transformation, the saturated cultures are centrifuged and bacterial pellets are re-suspended in Infiltration Media (0.5×MS, 1× B5 Vitamins, 5% sucrose, 1 mg/ml benzylaminopurine riboside, 200 μl/L Silwet L77) until an A600 reading of 0.8 is reached.

Transformation and seed harvest. The Agrobacterium solution is poured into dipping containers. All flower buds and rosette leaves of the plants are immersed in this solution for 30 seconds. The plants are laid on their side and wrapped to keep the humidity high. The plants are kept this way overnight at 4° C. and then the pots are turned upright, unwrapped, and moved to the growth racks.

The plants are maintained on the growth rack under 24-hour light until seeds are ready to be harvested. Seeds are harvested when 80% of the siliques of the transformed plants are ripe (approximately 5 weeks after the initial transformation). This transformed seed is deemed T0 seed, since it is obtained from the T0 generation, and is later plated on selection plates (either kanamycin or sulfonamide). Resistant plants that are identified on such selection plates comprised the T1 generation.

### Example IV

### Morphology

Morphological analysis is performed to determine whether changes in polypeptide levels affect plant growth and development. This is primarily carried out on the T1 generation, when at least 10-20 independent lines are examined. However, in cases where a phenotype requires confirmation or detailed characterization, plants from subsequent generations are also analyzed.

Primary transformants are typically selected on MS medium with 0.3% sucrose and 50 mg/l kanamycin. T2 and later generation plants are selected in the same manner, except that kanamycin is used at 35 mg/l. In cases where lines carry a sulfonamide marker (as in all lines generated by super-transformation), transformed seeds are selected on MS medium with 0.3% sucrose and 1.5 mg/l sulfonamide. KO lines are usually germinated on plates without a selection. Seeds are cold-treated (stratified) on plates for three days in the dark (in order to increase germination efficiency) prior to transfer to growth cabinets. Initially, plates are incubated at 22° C. under a light intensity of approximately 100 microEinsteins for 7 days. At this stage, transformants are green, possess the first two true leaves, and are easily distinguished from bleached kanamycin or sulfonamide-susceptible seedlings. Resistant seedlings are then transferred onto soil (e.g., Sunshine potting mix). Following transfer to soil, trays of seedlings are covered with plastic lids for 2-3 days to maintain humidity while they become established. Plants are grown on soil under fluorescent light at an intensity of 70-95 microEinsteins and a temperature of 18-23° C. Light conditions consist of a 24-hour photoperiod unless otherwise stated. In instances where alterations in flowering time is apparent, flowering time may be re-examined under both 12-hour and 24-hour light to assess whether the phenotype is photoperiod dependent. Under our 24-hour light growth conditions, the typical generation time (seed to seed) is approximately 14 weeks.

Because many aspects of Arabidopsis development are dependent on localized environmental conditions, in all cases plants are evaluated in comparison to controls in the same flat. As noted below, controls for transformed lines are wild-type plants or transformed plants harboring an empty nucleic acid construct selected on kanamycin or sulfonamide. Careful examination is made at the following stages: seedling (1 week), rosette (2-3 weeks), flowering (4-7 weeks), and late seed set (8-12 weeks). Seed is also inspected. Seedling morphology is assessed on selection plates. At all other stages, plants are macroscopically evaluated while growing on soil. All significant differences (including alterations in growth rate, size, leaf and flower morphology, coloration, and flowering time) are recorded, but routine measurements are not taken if no differences are apparent. In certain cases, stem sections are stained to reveal lignin distribution. In these instances, hand-sectioned stems are mounted in phloroglucinol saturated 2M HCl (which stains lignin pink) and viewed immediately under a dissection microscope.

Note that for a given transformation construct, up to ten lines may typically be examined in subsequent experimentation.

Analyses of light-mediated morphological changes: Light exerts its influence on many aspects of plant growth and development, including hypocotyl length, petiole length and petiole angle. Light triggers inhibition of hypocotyl elongation along with greening in young seedlings during photomorphogenesis. Mutant plants carrying functionally disruptive lesions in light signaling pathways generally have elongated hypocotyls, elongated petioles and altered petiole angle. For example, seedlings overexpressing G1988 exhibit elongated hypocotyls and elongated petioles compared to the control plants in light. The G1988 overexpressors are hyposensitive to blue, red and far-red wavelengths, indicating that G1988 acts downstream of the photoreceptors responsible for perceiving the different colors of light. It has been shown that hy5 and sth2 mutant seedlings, and COP1-OEX seedlings have elongated hypocotyls (Koornneef et al., 1980; McNellis et al., 1994b; Datta et al., 2007). The hypocotyl length measurements are performed on 4 to 7 day old seedlings grown on MS media plates as described above. The seedlings are grown under various light conditions; either white fluorescent light or monochromatic red, blue or far-red emitting LED lights. The hypocotyls are measured from digital photographs using ImageJ (freeware, NIH). Petiole length and petiole angles are measured from digital images (using ImageJ) of older plants grown in soil.
Root Growth Assay: Light signaling pathways can cause changes in root growth, architecture and root gravitropism. Seedlings are grown on MS media plates in white light for 10 to 15 days and analyzed for root growth and architecture. Digital images of roots can be used to quantify the number of lateral roots and root area. The angle of root growth is measured to determine the root gravitational response in comparison to the wild-type response.
Anthocyanin and other pigment measurements: Levels of anthocyanin and other colored pigments can often be visually assessed. For more quantitative measurements, the following procedure can be applied; seedlings grown on MS media plates for 4 to 7 days or leaves or other tissue materials from older plants are weighed and frozen in liquid nitrogen. Total plant pigments are extracted overnight in 1% HCl in methanol. The total pigments can be analyzed by HPLC. Anthocyanin can be partitioned from the mixture of total pigments by extraction of the mixture with a 1:1 mixture of chloroform and water. Anthocyanins are quantified spectrophotometrically from the upper (aqueous) phase (A₅₃₀-A₆₅₇) and normalized to fresh weight (Shin et al., 2007).

### Example V

### Methods to Determine Improved Plant Performance

In subsequent Examples, unless otherwise indicted, morphological and physiological traits are disclosed in comparison to wild-type control plants. That is, for example, a transformed or knockout/knockdown plant that is described as large and/or drought tolerant is large and more tolerant to drought with respect to a control plant, the latter including wild-type plants, parental lines and lines transformed with an “empty” nucleic acid construct that does not contain a polynucleotide sequence of interest (the sequence of interest is introduced into an experimental plant). When a plant is said to have a better performance than controls, it generally is larger, has greater yield, and/or shows less stress symptoms than control plants. The better performing lines may, for example, produce less anthocyanin, or are larger, greener, or more vigorous in response to a particular stress, as noted below. Better performance generally implies greater size or yield, or tolerance to a particular biotic or abiotic stress, less sensitivity to ABA, or better recovery from a stress (as in the case of a soil-based drought treatment) than controls. Improved performance can also be assessed by, for example, comparing the weight, volume, or quality of seeds, fruit, or other harvested plant parts obtained from an experimental plant (or population of experimental plants) compared to a control plant (or population of control plants).

A. Plate-Based Stress Tolerance Assays. Different plate-based physiological assays (shown below), representing a variety of abiotic and water-deprivation-stress related conditions, are used as a pre-screen to identify top performing lines (i.e. lines from transformation with a particular construct), that are generally then tested in subsequent soil based assays.

In addition, transgenic lines are may be subjected to nutrient limitation studies. A nutrient limitation assay is intended to find genes that allow more plant growth upon deprivation of nitrogen. Nitrogen is a major nutrient affecting plant growth and development that ultimately impacts yield and stress tolerance. These assays monitor primarily root but also rosette growth on nitrogen deficient media. In all higher plants, inorganic nitrogen is first assimilated into glutamate, glutamine, aspartate and asparagine, the four amino acids used to transport assimilated nitrogen from sources (e.g. leaves) to sinks (e.g. developing seeds). This process is regulated by light, as well as by C/N metabolic status of the plant. A C/N sensing assay is thus used to look for alterations in the mechanisms plants use to sense internal levels of carbon and nitrogen metabolites which could activate signal transduction cascades that regulate the transcription of N-assimilatory genes. To determine whether these mechanisms are altered, we exploit the observation that wild-type plants grown on media containing high levels of sucrose (3%) without a nitrogen source accumulate high levels of anthocyanins. This sucrose induced anthocyanin accumulation can be relieved by the addition of either inorganic or organic nitrogen. We use glutamine as a nitrogen source since it also serves as a compound used to transport N in plants.

Germination assays. The following germination assays are typically conducted with Arabidopsis knockdowns/knockouts or overexpression lines: NaCl (150 mM), mannitol (300 mM), sucrose (9.4%), ABA (0.3 μM), cold (8° C.), polyethlene glycol (10%, with Phytogel as gelling agent), or C/N sensing or low nitrogen medium. In the text below, —N refers to basal media minus nitrogen plus 3% sucrose and −N/+Gln is basal media minus nitrogen plus 3% sucrose and 1 mM glutamine.

All germination assays are performed in tissue culture. Growing the plants under controlled temperature and humidity on sterile medium produces uniform plant material that has not been exposed to additional stresses (such as water stress) which could cause variability in the results obtained. All assays are designed to detect plants that are more tolerant or less tolerant to the particular stress condition and are developed with reference to the following publications: Jang et al., 1997; Smeekens, 1998; Liu and Zhu, 1997; Saleki et al., 1993; Wu et al., 1996; Zhu et al., 1998; Alia et al., 1998; Xin and Browse, 1998; Leon-Kloosterziel et al., 1996. Where possible, assay conditions are originally tested in a blind experiment with controls that had phenotypes related to the condition tested.

Prior to plating, seed for all experiments are surface sterilized in the following manner: (1) 5 minute incubation with mixing in 70% ethanol, (2) 20 minute incubation with mixing in 30% bleach, 0.01% triton-X 100, (3) 5× rinses with sterile water, (4) Seeds are re-suspended in 0.1% sterile agarose and stratified at 4° C. for 3-4 days.

All germination assays follow modifications of the same basic protocol. Sterile seeds are sown on the conditional media that has a basal composition of 80% MS+Vitamins. Plates are incubated at 22° C. under 24-hour light (120-130 μm⁻² s⁻¹) in a growth chamber. Evaluation of germination and seedling vigor is performed five days after planting.

Growth assays. The following growth assays are typically conducted with Arabidopsis knockdowns/knockouts or overexpression lines: severe desiccation (a type of water deprivation assay), growth in cold conditions at 8° C., root development (visual assessment of lateral and primary roots, root hairs and overall growth), and phosphate limitation. For the nitrogen limitation assay, plants are grown in 80% Murashige and Skoog (MS) medium in which the nitrogen source is reduced to 20 mg/L of NH₄NO₃. Note that 80% MS normally has 1.32 g/L NH₄NO₃ and 1.52 g/L KNO₃. For phosphate limitation assays, seven day old seedlings are germinated on phosphate-free medium in MS medium in which KH₂PO₄ is replaced by K₂SO₄.

Unless otherwise stated, all experiments are performed with the Arabidopsis thaliana ecotype Columbia (Col-0). Similar assays could be devised for other crop plants such as soybean or maize plants. Assays are usually conducted on non-selected segregating T2 populations (in order to avoid the extra stress of selection). Control plants for assays on lines containing direct promoter-fusion constructs are Col-0 plants transformed an empty transformation nucleic acid construct (pMEN65). Controls for 2-component lines (generated by supertransformation) are the background promoter-driver lines (i.e. promoter::LexA-GAL4TA lines), into which the supertransformations are initially performed.

### Procedures

For chilling growth assays, seeds are germinated and grown for seven days on MS+Vitamins+1% sucrose at 22° C. and then transferred to chilling conditions at 8° C. and evaluated after another 10 days and 17 days.

For severe desiccation (plate-based water deprivation) assays, seedlings are grown for 14 days on MS+Vitamins+1% Sucrose at 22° C. Plates are opened in the sterile hood for 3 hr for hardening and then seedlings are removed from the media and dried for two hours in the sterile hood. After this time, the plants are transferred back to plates and incubated at 22° C. for recovery. The plants are then evaluated after five days.

For a polyethylene glycol (PEG) hyperosmotic stress tolerance screen, plant seeds are gas sterilized with chlorine gas for 2 hrs. The seeds are plated on each plate containing 3% PEG, ½×MS salts, 1% phytagel, and antibiotic or herbicide selection if appropriate. Two replicate plates per seedline are planted. The plates are placed at 4° C. for 3 days to stratify seeds. The plates are held vertically for 11 additional days at temperatures of 22° C. (day) and 20° C. (night). The photoperiod is 16 hrs. with an average light intensity of about 120 μmol/m2/s. The racks holding the plates are rotated daily within the shelves of the growth chamber carts. At 11 days, root length measurements are made. At 14 days, seedling status is determined, root length is measured, growth stage is recorded, the visual color is assessed, pooled seedling fresh weight is measured, and a whole plate photograph is taken.

Data interpretation. At the time of evaluation, plants are typically given one of the following qualitative scores, based upon a visual inspection:
(++) Substantially enhanced performance compared to controls. The phenotype is very consistent and growth is significantly above the normal levels of variability observed for that assay.
(+) Enhanced performance compared to controls. The response is consistent but is only moderately above the normal levels of variability observed for that assay.
(wt) No detectable difference from wild-type controls.
(−) Impaired performance compared to controls. The response is consistent but is only moderately below the normal levels of variability observed for that assay.
(−−) Substantially impaired performance compared to controls. The phenotype is consistent and growth is significantly below the normal levels of variability observed for that assay.
(n/d) Experiment failed, data not obtained, or assay not performed.

### B. Estimation of Water Use Efficiency (WUE).

An aspect of this invention provides transgenic plants with enhanced yield resulting from enhanced water use efficiency and/or water deprivation tolerance. WUE can be estimated through isotope discrimination analysis, which exploits the observation that elements can exist in both stable and unstable (radioactive) forms. Most elements of biological interest (including C, H, O, N, and S) have two or more stable isotopes, with the lightest of these present in much greater abundance than the others. For example, ¹²C is more abundant than ¹³C in nature (¹²C=98.89%, ¹³C=1.11%, ¹⁴C=<10-10%). Because ¹³C is slightly larger than ¹²C, fractionation of CO₂ during photosynthesis occurs at two steps:

1. ¹²CO₂ diffuses through air and into the leaf more easily;

2. ¹²CO₂ is preferred by the enzyme in the first step of photosynthesis, ribulose bisphosphate carboxylase/oxygenase.

WUE has been shown to be negatively correlated with carbon isotope discrimination during photosynthesis in several C3 crop species. Carbon isotope discrimination has been linked to drought tolerance and yield stability in drought-prone environments and has been successfully used to identify genotypes with better drought tolerance. ¹³C/¹²C content is measured after combustion of plant material and conversion to CO₂, and analysis by mass spectroscopy. With comparison to a known standard, ¹³C content may be altered in such a way as to suggest that altering expression of HY5, STH2, COP1 or closely related sequences improves water use efficiency.

Another parameter correlated with WUE is stomatal conductance. Changes in stomatal conductance regulate CO₂ and H₂O exchange between the leaf and the atmosphere and can be determined from measurements of H₂O loss from a leaf made in an infra-red gas analyzer (LI-6400, Licor Biosciences, Lincoln, Nebr.). The rate of H₂O loss from a leaf is calculated from the difference between the H₂O concentration of air flowing over a leaf and air flowing through an empty reference cell. The H₂O concentration in both the reference and sample cells is determined from the absorption of infra-red radiation by the H₂O molecules.

A third method for estimating water use efficiency is to grow a plant in a known amount of soil and water in a container in which the soil is covered to prevent water evaporation, e.g. by a lid with a small hole [for one example, see Nienhuis et al. (1994)]. Water use efficiency is calculated by taking the fresh or dry plant weight after a given period of growth, and dividing by the weight of water used. The amount of water lost by transpiration through the plant is estimated by subtracting the final weight of the container and soil from the initial weight.

### C. Analysis of Water Deprivation (Drought) Tolerance

An aspect of this invention provides transgenic plants with enhanced yield resulting from enhanced water use efficiency and/or water deprivation tolerance. A number of screening methods can be used to assess water deprivation tolerance; sample methods are described below.

### (i) Clay Pot Based Soil Drought Assay for Arabidopsis Plants

This soil drought assay (performed in clay pots) is based on that described by Haake et al., 2002.

Experimental Procedure. Seeds are sterilized by a 2 minute ethanol treatment followed by 20 minutes in 30% bleach/0.01% Tween and five washes in distilled water. Seeds are sown to MS agar in 0.1% agarose and stratified for three days at 4° C., before transfer to growth cabinets with a temperature of 22° C. After seven days of growth on selection plates, seedlings are transplanted to 3.5 inch diameter clay pots containing 80 g of a 50:50 mix of vermiculite:perlite topped with 80 g of ProMix. Typically, each pot contains 14 seedlings, and plants of the transformed line being tested are in separate pots to the wild-type controls. Pots containing the transgenic line versus control pots are interspersed in the growth room, maintained under 24-hour light conditions (18-23° C., and 90-100 μE m⁻² s⁻¹) and watered for a period of 14 days. Water is then withheld and pots are placed on absorbent paper for a period of 8-10 days to apply a drought treatment. After this period, a visual qualitative “drought score” from 0-6 is assigned to record the extent of visible drought stress symptoms. A score of “6” corresponds to no visible symptoms whereas a score of “0” corresponds to extreme wilting and the leaves having a “crispy” texture. At the end of the drought period, pots are re-watered and scored after 5-6 days; the number of surviving plants in each pot is counted, and the proportion of the total plants in the pot that survived is calculated.

Analysis of results. In a given experiment, six or more pots of a transformed line are typically compared with six or more pots of the appropriate control. The mean drought score and mean proportion of plants surviving (survival rate) are calculated for both the transformed line and the wild-type pots. In each case a p-value* is calculated, which indicates the significance of the difference between the two mean values. The results for each transformed line across each planting for a particular project are then presented in a results table.

Calculation of p-values. For the assays where control and experimental plants are in separate pots, survival is analyzed with a logistic regression to account for the fact that the random variable is a proportion between 0 and 1. The reported p-value is the significance of the experimental proportion contrasted to the control, based upon regressing the logit-transformed data.

Drought score, being an ordered factor with no real numeric meaning, is analyzed with a non-parametric test between the experimental and control groups. The p-value is calculated with a Mann-Whitney rank-sum test.

### (ii) Wilt Screen Assay for Soybean Plants

Transformed and wild-type soybean plants are grown in 5″ pots in growth chambers. After the seedlings reach the V1 stage (the V1 stage occurs when the plants have one trifoliate, and the unifoliate and first trifoliate leaves are unrolled), water is withheld and the drought treatment thus started. A drought injury phenotype score is recorded, in increasing severity of effect, as 1 to 4, with 1 designated no obvious effect and 4 indicating a dead plant. Drought scoring is initiated as soon as one plant in one growth chamber has a drought score of 1.5. Scoring continues every day until at least 90% of the wild type plants achieve scores of 3.5 or more. At the end of the experiment the scores for both transgenic and wild type soybean seedlings are statistically analyzed using Risk Score and Survival analysis methods (Glantz, 2001; Hosmer and Lemeshow, 1999).

(iii) Greenhouse Screening for Water Deprivation Tolerance and/or Water Use Efficiency

This example describes a high-throughput method for greenhouse selection of transgenic maize plants compared to wild type plants (tested as inbreds or hybrids) for water use efficiency. This selection process imposes three drought/re-water cycles on the plants over a total period of 15 days after an initial stress free growth period of 11 days. Each cycle consists of five days, with no water being applied for the first four days and a water quenching on the fifth day of the cycle. The primary phenotypes analyzed by the selection method are the changes in plant growth rate as determined by height and biomass during a vegetative drought treatment. The hydration status of the shoot tissues following the drought is also measured. The plant heights are measured at three time points. The first is taken just prior to the onset drought when the plant is 11 days old, which is the shoot initial height (SIH). The plant height is also measured halfway throughout the drought/re-water regimen, on day 18 after planting, to give rise to the shoot mid-drought height (SMH). Upon the completion of the final drought cycle on day 26 after planting, the shoot portion of the plant is harvested and measured for a final height, which is the shoot wilt height (SWH) and also measured for shoot wilted biomass (SWM). The shoot is placed in water at 40° C. in the dark. Three days later, the weight of the shoot is determined to provide the shoot turgid weight (STM). After drying in an oven for four days, the weights of the shoots are determined to provide shoot dry biomass (SDM). The shoot average height (SAH) is the mean plant height across the three height measurements. If desired, the procedure described above may be adjusted for +/−approximately one day for each step. To correct for slight differences between plants, a size corrected growth value is derived from SIH and SWH. This is the Relative Growth Rate (RGR). Relative Growth Rate (RGR) is calculated for each shoot using the formula [RGR %=(SWH−SiH)/((SWH+SiH)/2)*100]. Relative water content (RWC) is a measurement of how much (%) of the plant is water at harvest. Water Content (RWC) is calculated for each shoot using the formula [RWC %=(SWM−SDM)/(STM−SDM)*100]. For example, fully watered corn plants of this stage of development have around 98% RWC.

### D. Measurement of Photosynthesis.

Photosynthesis is measured using an infra red gas analyzer (LICOR LI-6400, Li-Cor Biosciences, Lincoln, Nebr.). The measurement technique is based on the principle that because CO₂ absorbs infra-red radiation, the CO2 concentration of different air streams can be determined from changes in absorption of infra-red radiation. Because photosynthesis is the process of converting CO₂ to carbohydrates, we expect to see a decrease in the amount of CO₂ in air flowing over a leaf relative to a reference air stream without a leaf. From this difference, given a known air flow rate and leaf area, a photosynthesis rate can be calculated. In some cases, respiration will increase the CO2 concentration in the air stream flowing over the leaf relative to the reference air stream. To perform measurements, the LI-6400 is set-up and calibrated as per LI-6400 standard directions. Photosynthesis can then be measured over a range of light levels and atmospheric CO₂ and H₂O concentrations.

Fluorescence of absorbed light from chlorophyll a molecules in the leaf is one pathway by which light energy absorbed by the leaf can be dissipated. As such, measurement of chlorophyll a fluorescence is used to measure changes in photochemistry and photoprotection, the main pathways by which absorbed light energy is dissipated by a leaf. A fluorimeter (e.g. the LI6400-40, Licor Biogeosciences, Lincoln, Nebr.; or the OS-1, Opti Sciences, Hudson, N.H.) can be used to measure the fate of absorbed light for leaves over a range of growth and experimental conditions in accordance with the manufacturer's guidelines.

### Example VI

### Phenotypes Conferred by G1988-Related Genes

Tables 5 and 6 list some of the morphological and physiological traits, respectively, obtained in Arabidopsis, soy or corn plants overexpressing G1988 or orthologs from diverse species of plants, including Arabidopsis, soy, maize, rice, and tomato, in experiments conducted to date. All observations are made with respect to control plants that did not overexpress a G1988 clade transcription factor.

TABLE 6
G1988 homologs and potentially valuable development-related traits

| | Col. 2 | | | Col. 5 |
|---|---|---|---|---|
| Col. 1 | Reduced light response: | | Col. 4 | Altered |
| GID | elongated hypocotyls, | Col. 3 | Increased | development and/or |
| (SEQ ID No.) | elongated petioles or | Increased | secondary | time to flowering |
| Species | upright leaves | yield* | roots | observed |
| G1988 (28) At | +¹ | +³ | +¹ | +^{1,3} |
| G4004 (30) Gm | +¹ | | n/d | +¹ |
| G4005 (32) Gm | +¹ | n/d* | n/d | +¹ |
| G4000 (44) Zm | +¹ | n/d* | n/d | +¹ |
| G4011 (34) Os | +¹ | n/d* | n/d | |
| G4012 (36) Os | +¹ | n/d* | n/d | +¹ |
| G4299 (42) Sl | +¹ | n/d* | n/d | +¹ |

*yield may be increased by morphological improvements, developmental improvements, physiological improvements such as enhanced photosynthesis, and/or increased tolerance to various physiological stresses; based on the beneficial effects of G1988 clade member overexpression on light response and abiotic stress tolerance listed in Tables 5 and 6, it is expected that overexpression of other G1988 clade member polypeptides will result in increased yield in commercial plant species.

TABLE 7
Effects of G1988 and closely related homologs on
physiological traits and abiotic stress tolerance

| Col. 1 | Col. 2 | Col. 3 | Col. 4 | Col. 5 |
|---|---|---|---|---|
| GID | Better | Increased | Altered | Increased |
| (SEQ ID No.) | germination in | water deprivation | C/N sensing or | hyperosmotic stress |
| Species | cold conditions | tolerance | low N tolerance | (sucrose) tolerance |
| G1988 (28) At | +³ | +^{1,3} | +¹ | +¹ |
| G4004 (30) Gm | +^{1,2,3} | | +^{1,2} | +¹ |
| G4005 (32) Gm | +¹ | | +¹ | +¹ |
| G4000 (44) Zm | −¹ | n/d | +¹ | n/d |
| G4011 (34) Os | +¹ | n/d | +¹ | +¹ |
| G4012 (36) Os | +¹ | n/d | +¹ | +¹ |
| G4299 (42) Sl | +¹ | n/d | +¹ | +¹ |

Notes and abbreviations for Tables 5 and 6:
At—Arabidopsis thaliana; Gm—Glycine max; Os—Oryza sativa; Sl—Solanum lycopersicum; Zm—Zea mays
(+) indicates positive assay result/more tolerant or phenotype observed, relative to controls.
(−) indicates negative assay result/less tolerant or phenotype observed, relative to controls
empty cell—assay result similar to controls
¹phenotype observed in Arabidopsis plants
²phenotype observed in maize plants, as disclosed in U.S. patent application No. US20080010703
³phenotype observed in soy plants, as disclosed in U.S. patent application No. US20080010703
n/d—assay not yet done or completed
N—Altered C/N sensing or low nitrogen tolerance
Water deprivation tolerance was indicated in soil-based drought or plate-based desiccation assays
Hyperosmotic stress was indicated by greater tolerance to 9.4% sucrose than controls
Increased cold tolerance was indicated by greater tolerance to 8° C. during germination or growth than controls
Altered C/N sensing or low nitrogen tolerance assays were conducted in basal media minus nitrogen plus 3% sucrose or basal media minus nitrogen plus 3% sucrose and 1 mM glutamine; for the nitrogen limitation assay, the nitrogen source of 80% MS medium was reduced to 20 mg/L of NH₄NO₃.
A reduced light sensitivity phenotype was indicated by longer petioles, longer hypocotyls and/or upturned leaves relative to control plants
n/d—assay not yet done or completed

### Example VII

### Manipulation of G1988 Pathway Components to Improve Stress Tolerance

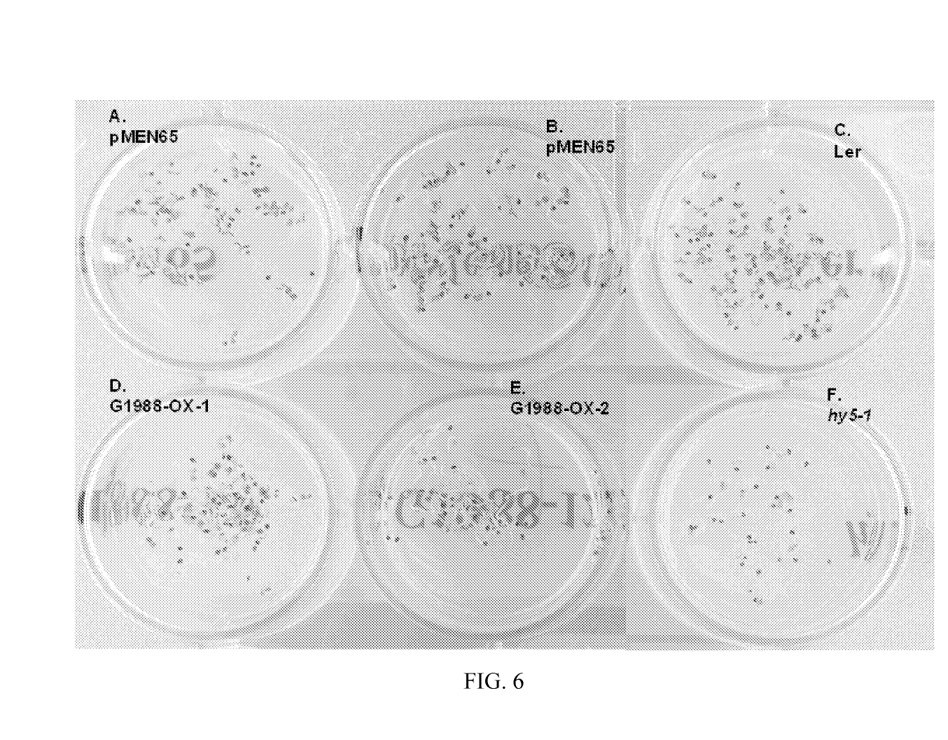

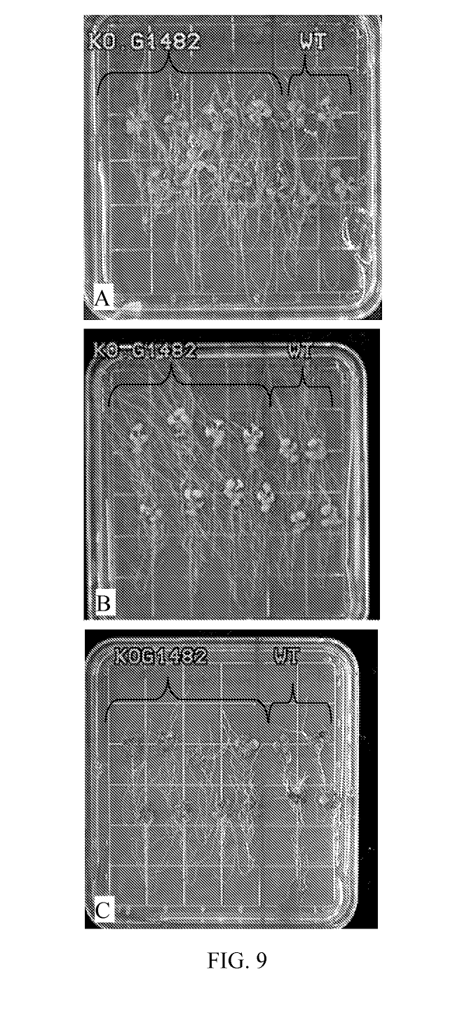

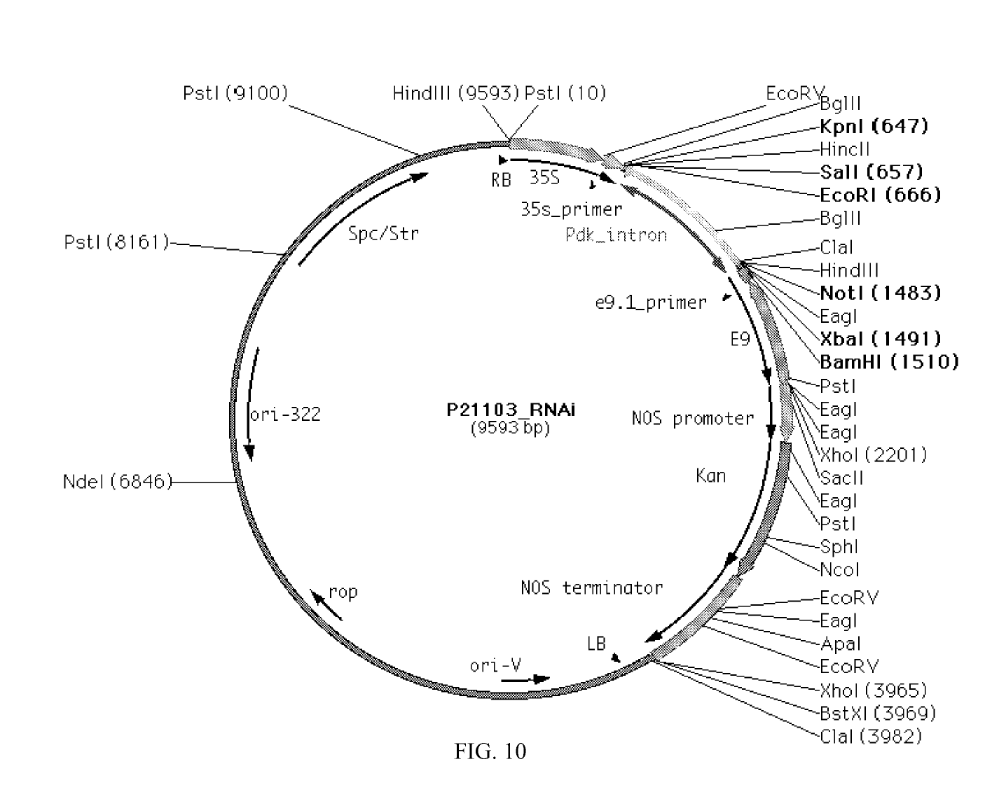

It is known that HY5, SEQ ID NO: 2, is involved in photomorphogenesis (Koornneef et al., 1980; Ang and Deng, 1994; Somers et al., 1991; Shin et al., 2007). As described below, G1988, SEQ ID NO: 28, overexpressing seedlings are hyposensitive to light and have elongated hypocotyls. The first test to determine whether a reduction in HY5 activity produces similar positive effects on abiotic stress tolerance to G1988 overexpression was performed. For this experiment we made use of the hy5-1 mutant, which lacks a functional HY5 protein (obtained from ABRC, Ohio and originally described by Koornneef et al., 1980). In these experiments, the accumulation of anthocyanin was used as a “read-out” of the stress tolerance of the seedlings. Seedlings were subjected to germination assays comprising a pair of C/N sensing assays (Hsieh et al., 1998) and a sucrose tolerance assay (the latter represented an osmotic stress). For the C/N sensing assays, seeds were germinated on either of two types of plates: (i) comprising MS salt mix, and 3% sucrose, but lacking nitrogen (N—) or (ii) MS salt mix, and 3% sucrose but containing 1 mM Glutamine (N−/gln) as a nitrogen source. The sucrose tolerance assay plates contained complete basal salt mix with nitrogen and contained 9.4% sucrose. Representative results are shown in FIG. 6. The experiment compared the C/N (Carbon/Nitrogen) sensitivity of two G1988 overexpressors (G1988-OX-1 and G1988-OX-2, FIGS. 6D and 6E) with their respective wild-type controls (pMEN65, which are Columbia transformed with the empty backbone vector used for G1988-OX lines, FIGS. 6A and 6B), and we compared the hy5-1 mutant (FIG. 6F) with its wild-type control, Ler (FIG. 6C). All of the wild-type controls accumulated more anthocyanin than the hy5-1 and G1988-OX seedlings when grown on N− plates. Three biological replicates were scored visually for green color (designated as “+”) compared to their respective wild-type seedlings and it was found that the G1988-OX seedlings behaved like hy5-1 mutants and accumulated less anthocyanin than the wild-type controls under all conditions tested. These data provide a second phenotypic comparison between the G1988 overexpressors and hy5-1 seedlings. It appears that G1988 and HY5 function antagonistically to each other in regulating hypocotyl elongation and stress responses. Furthermore, our studies with STH2 overexpressing lines have shown that like HY5, STH2 overexpression acts to increase anthocyanin levels compared to wild type controls. STH2 (SEQ ID NO: 24) was recently shown to bind HY5 and to function with HY5 (Datta et. al., 2007). We have further shown that plants of a knockout line homozygous for a T-DNA insertion at approximately 400 bp downstream of the STH2 (G1482) start codon are more tolerant to abiotic stress; seedlings from this sth2 T-DNA line showed increased tolerance to osmotic and low nutrient conditions as indicated by more vigorous growth (including root growth) compared to wild-type control plants in the same experiments (FIG. 9).

### Example VIII

### G1988 Overexpression or a hy5 Mutation Affect the Light-Regulated Expression of Common Downstream Target Genes Indicating that they Function in the Same Pathway

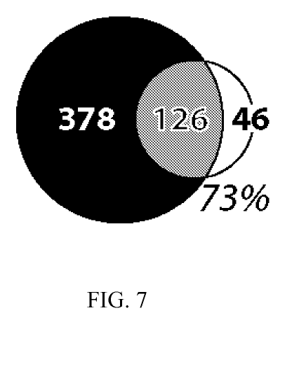

Plants are sensitive to light direction, quantity and quality. Approximately 10% of Arabidopsis genes respond to the informational light signal. Red, blue and far-red wavelengths are perceived by photosensory photoreceptors and the signal is transmitted downstream through a network of master transcription factors (Tepperman et al., 2001). HY5 is thought to function at a higher hierarchical level at the point of convergence of these different light signaling pathways (Osterlund, 2000). Previously we have shown that the B-box containing factor G1988 functions negatively in the phototransduction pathway and its overexpression confers higher broad acre yield in soybeans along with other beneficial traits (see US Patent Application No. US20080010703A1). It is expected that G1988 and HY5 function antagonistically to each other in the same phototransduction pathway. In order to test this hypothesis, we performed microarray based transcription profiling of G1988-OEX and hy5-1 mutant seedlings, which were either grown in darkness or were exposed to 1 h or 3 h of monochromatic red irradiation. Global gene expression profiling revealed that at the 1 h time point (after lights on), G1988 and HY5 have a significant overlap in target gene regulation; they act upstream of the same 42.3% of all light responsive genes (FIG. 7). Both G1988-OEX and hy5-1 mutants exhibited reduced light responsivity, indicating that they act antagonistically. It is expected that G1988 acts to repress HY5 activity. Down regulation or knockout approaches on the activity or expression of HY5 and related proteins will result in similar or greater crop benefits as conferred by G1988 overexpression. Furthermore, since another B-box protein, G1482 (STH2), is known to function positively in HY5 mediated signaling (Datta et al., 2007), we expect that similar knockout or down regulation approaches with G1482 and its related proteins will result in improvement of crop traits. COP1 is known to regulate HY5 activity by rapidly degrading HY5; hence overexpression of COP1 and its related proteins will have the same effect. The data presented in FIG. 7 show that these proteins regulate the same pathway as G1988 and altering their activities (either increasing or decreasing) within crop plants will produce desired effects in crop plants.

### Example IX

### Loss of HY5 Activity is Epistatic to the Loss of G1988 Activity in Regulating Hypocotyl Length in a g1988-1;hy5-1 Double Mutant

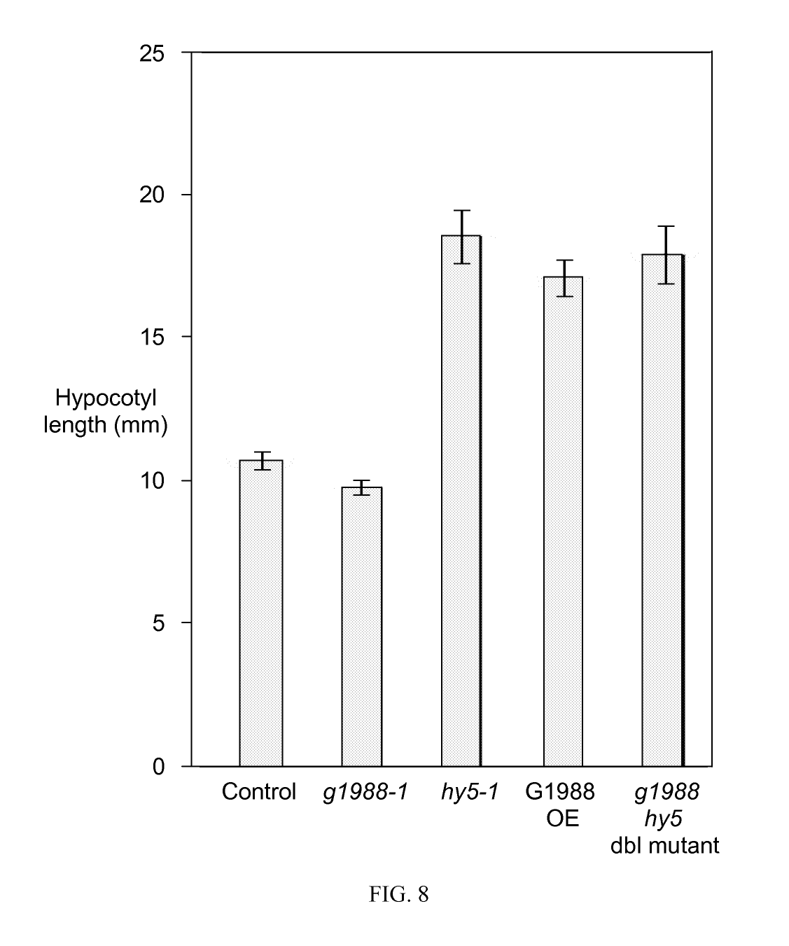

Previous experiments (described above) indicated that both G1988 and HY5 function in the phototransduction pathway and that G1988 possibly suppresses HY5 activity. In order to determine the genetic interaction (epistasis) between these two genes, we crossed the g1988-1 mutant (T-DNA insertional disruption mutant SALK_{—}059534, from ABRC (Arabidopsis Biological Resource Center)) with the hy5-1 mutant, and used a quantitative trait (hypocotyl length) as a marker. As seen in FIG. 8, after 7 days of growth in red light, the hypocotyls of WT control seedlings were about 10 mm long and the g1988-1 seedlings had hypocotyls slightly shorter than 10 mm, whereas the hy5-1 mutant, the G1988-OEX and the g1988-1;hy5-1 double mutants had hypocotyl lengths close to 17 mm long. These data show that hy5-1 has a dominant epistatic relationship with G1988. At the biochemical level, G1988 acts to increase hypocotyl length in light, whereas HY5 acts to suppress hypocotyl length. The absence of G1988 activity in the g1988-1 mutant has a marginal effect on hypocotyl length with HY5 activity at the wild type levels in these seedlings. However, in the g1988-1;hy5-1 double mutant, the loss of hy5-1 activity has a dominant effect resulting in long hypocotyls similar to the hy5-1 single mutant and the G1988-OEX seedlings (FIG. 8). These data, together with the array analyses suggest that G1988 acts to suppress HY5. Overexpression of G1988 causes broader, pleiotropic effects in crop plants; it is likely that reducing the levels of HY5 activity will provide a similar or greater yield advantage to G1988 with fewer or no undesired effects. A similar advantage may be achieved by reducing expression of STH2 (SEQ ID NO: 24, G1482) and related proteins, or increasing expression of COP1 (SEQ ID NO: 14, G1518) and related proteins.

### Example X

### Manipulation of HY5, STH2 and COP1 (SEQ ID NOs: 2, 24 and 14, Respectively) to Improve Yield

It is possible that altering COP1 activity will have broader effects, but altering HY5 activity will allow a more targeted approach. Furthermore, a recent study with STH2 (SEQ ID NO: 24, G1482) has indicated that this B-box protein functions with HY5 to promote phototransduction (Datta et al., 2007). It is very likely that alteration of STH2 activity may provide similar results in crop plants.

The current invention utilizes methods to knockdown/knockout the activity of HY5 or STH2, (SEQ ID NOs: 2 or 24), or their closely-related homologs (e.g., SEQ ID NOs: 4, 6, 8, 10, 12, 26, 48 or 50); or overexpress COP1 (SEQ ID NO 14), or its closely-related homologs (e.g., SEQ ID NOs: 16, 18, 20 or 22), to create transgenic plants that are hyposensitive to light, which will improve performance or yield in crops like soybean. Furthermore, altering the activity of HY5, STH2, COP1, or of their closely related homologs during a specific phase of the photoperiod using a promoter element that is active at a particular time of day is likely to provide the benefits and prevent undesired effects. Examples of putative HY5, COP1 and STH2 homologs which are considered suitable targets for such approaches are provided in the Sequence Listing. Because light signaling pathways are conserved in plants, it is envisioned that beneficial traits will be achieved in a wide range of commercial crops, including but not limited to soybean, canola, corn, rice, cotton, tree species, forage, turf grasses, fruits, vegetables, ornamentals and biofuel crops such as, for example, switchgrass or Miscanthus.

Suppression of the activity of HY5 or STH2 (SEQ ID NOs: 2 or 24), or their closely related homologs (e.g., SEQ ID NOs: 4, 6, 8, 10, 12, 26, 48 or 50), can be achieved by various methods, including but not limited to co-suppression, chemical mutagenesis, fast neutron deletions, X-rays, antisense strategies, RNAi based approaches, targeted gene silencing, virus induced gene silencing (VIGS), molecular breeding, TILLING (McCallum et al., 2000), overexpression of suppressors of HY5 (like COP1), or the overexpression of microRNAs that target HY5 or STH2. Further methods could be applied, which rely on introducing a DNA molecule into a plant cell, which is engineered to induce changes at an endogenous HY5 (or COP1 or STH2) related locus through a homology dependent DNA-repair or recombination based process. Such “gene replacement” approaches are routine in systems such as yeast and are now being developed for use in plants. An increase in COP1 (SEQ ID NO: 14), or its closely related homologs (e.g., SEQ ID NOs: 16, 18, 20 or 22) activity in soybean, can be achieved by transgenic approaches resulting in gene overexpression or by suppression of negative regulators of these genes by one or more approaches discussed above.

### Example XI

### Utilities of HY5 and STH2 (and Related Sequence) Suppression Lines

HY5 and STH2 suppression lines and COP1 overexpression lines may be created by using either a constitutive promoter or a promoter with activity at a specific time of day, or with activity targeted to particular developmental stage or tissue, as described above. Yield advantage and other beneficial traits will be achieved in a wide range of commercial crops, including but not limited to soybean, corn, rice and cotton. Since light signaling pathways share common signaling mechanisms in plants, this approach will be applicable for one or more forestry, forage, turf, fruits, vegetables, ornamentals or biofuel crops.

### Example XII

### Transformation of Dicots to Produce Increased Yield and/or Abiotic Stress Tolerance

Crop species that have reduced or knocked-out expression of polypeptides of the invention may produce plants with greater yield, greater height, increased secondary rooting, greater cold tolerance, greater tolerance to water deprivation, reduced stomatal conductance, altered C/N sensing, increased low nitrogen tolerance, increased tolerance to hyperosmotic stress, reduced percentage of hard seed, greater average stem diameter, increased stand count, improved late season growth or vigor, increased number of pod-bearing main-stem nodes, or greater late season canopy coverage, as compared to control plants, in both stressed and non-stressed conditions. Thus, polynucleotide sequences listed in the Sequence Listing recombined into, for example, one of the nucleic acid constructs of the invention, or another suitable expression vector, may be transformed into a plant for the purpose of modifying plant traits for the purpose of improving yield and/or quality. The expression vector may contain a constitutive, tissue-specific or inducible promoter operably linked to the polynucleotide. The cloning vector may be introduced into a variety of plants by means well known in the art such as, for example, direct DNA transfer or Agrobacterium tumefaciens-mediated transformation. It is now routine to produce transgenic plants using most dicot plants (see Weissbach and Weissbach, 1989; Gelvin et al. 1990; Herrera-Estrella et al., 1983; Bevan, 1984; and Klee, 1985). Methods for analysis of traits are routine in the art and examples are disclosed above.

Numerous protocols for the transformation of tomato and soy plants have been previously described, and are well known in the art. Gruber et al., 1993, and Glick and Thompson, 1993 describe several nucleic acid constructs and culture methods that may be used for cell or tissue transformation and subsequent regeneration. For soybean transformation, methods are described by Miki et al., 1993; and U.S. Pat. No. 5,563,055 to Townsend and Thomas. For efficient transformation of canola, examples of methods have been reported by Cardoza and Stewart, 1992.

There are a substantial number of alternatives to Agrobacterium-mediated transformation protocols, other methods for the purpose of transferring exogenous genes into soybeans or tomatoes. One such method is microprojectile-mediated transformation, in which DNA on the surface of microprojectile particles is driven into plant tissues with a biolistic device (see, for example, Sanford et al., 1987; Christou et al., 1992; Sanford, 1993; Klein et al., 1987; U.S. Pat. No. 5,015,580 to Christou et al.; and U.S. Pat. No. 5,322,783 to Tomes et al.).

Alternatively, sonication methods (see, for example, Zhang et al., 1991); direct uptake of DNA into protoplasts using CaCl₂ precipitation, polyvinyl alcohol or poly-L-ornithine (see, for example, Hain et al., 1985; Draper et al., 1982); liposome or spheroplast fusion (see, for example, Deshayes et al., 1985; Christou et al., 1987); and electroporation of protoplasts and whole cells and tissues (see, for example, Donn et al., 1990; D'Halluin et al., 1992; and Spencer et al., 1994) have been used to introduce foreign DNA and nucleic acid constructs into plants.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and the traits they confer that have been introduced into a tomato or soybean line may be moved into distinct line of plants using traditional backcrossing techniques well known in the art. Transformation of tomato plants may be conducted using the protocols of Koornneef et al., 1986, and in U.S. Pat. No. 6,613,962 to Vos et al., the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing a nucleic acid construct comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD₆₀₀ of 0.8.

Following cocultivation, the cotyledon explants are transferred to Petri dishes with selective medium comprising MS medium with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulfate-containing medium is a positive indication of a successful transformation.

Transformation of soybean plants may be conducted using the methods found in, for example, U.S. Pat. No. 5,563,055 to Townsend et al., described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the nucleic acid construct comprising a polynucleotide of the invention are grown to log phase, pooled, and concentrated by centrifugation. Inoculations are conducted in batches such that each plate of seed is treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium that has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055 to Townsend et al.).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

### Example XIII

### Transformation of Monocots to Produce Increased Yield or Abiotic Stress Tolerance

Cereal plants such as, but not limited to, corn, wheat, rice, sorghum, or barley, may be transformed with the present polynucleotide sequences, including monocot or dicot-derived sequences such as those presented in the present Tables, cloned into a nucleic acid construct such as pGA643 and containing a kanamycin-resistance marker, and expressed constitutively under, for example, the CaMV 35S or COR15 promoters, or with tissue-specific or inducible promoters. The nucleic acid constructs may be one found in the Sequence Listing, or any other suitable expression vector may be similarly used. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The nucleic acid construct may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616 to Hiei and Komari, in which monocotyledon callus is transformed by contacting dedifferentiating tissue with the Agrobacterium containing the nucleic acid construct.

The sample tissues are immersed in a suspension of 3×10⁹ cells of Agrobacterium containing the nucleic acid construct for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from 5Prime-3Prime Inc. (Boulder, Colo.).

It is also routine to use other methods to produce transgenic plants of most cereal crops (Vasil, 1994) such as corn, wheat, rice, sorghum (Casas et al., 1993), and barley (Wan and Lemeaux, 1994). DNA transfer methods such as the microprojectile method can be used for corn (Fromm et al., 1990; Gordon-Kamm et al., 1990; Ishida, 1990), wheat (Vasil et al., 1992; Vasil et al., 1993; Weeks et al., 1993), and rice (Christou, 1991; Hiei et al., 1994; Aldemita and Hodges, 1996; and Hiei et al., 1997). For most cereal plants, embryogenic cells derived from immature scutellum tissues are the preferred cellular targets for transformation (Hiei et al., 1997; Vasil, 1994). For transforming corn embryogenic cells derived from immature scutellar tissue using microprojectile bombardment, the A188XB73 genotype is the preferred genotype (Fromm et al., 1990; Gordon-Kamm et al., 1990). After microprojectile bombardment the tissues are selected on phosphinothricin to identify the transgenic embryogenic cells (Gordon-Kamm et al., 1990). Transgenic plants are regenerated by standard corn regeneration techniques (Fromm et al., 1990; Gordon-Kamm et al., 1990).

### Example XIV

### Expression and Analysis of Increased Yield or Abiotic Stress Tolerance in Non-Arabidopsis Species

It is expected that structurally similar orthologs of the G557 (HY5), G1482 (STH2) and G1518 (COP1) clades of polypeptide sequences, including those found in the Sequence Listing, can confer increased yield or increased tolerance to a number of abiotic stresses, including water deprivation, cold, and low nitrogen conditions, relative to control plants, when the expression levels of these sequences are altered. It is also expected that these sequences can confer improved water use efficiency (WUE), increased root growth, and tolerance to greater planting density. As sequences of the invention have been shown to improve stress tolerance and other properties, it is also expected that these sequences will increase yield of crop or other commercially important plant species.

Northern blot analysis, RT-PCR or microarray analysis of the regenerated, transformed plants may be used to show expression of a polypeptide or the invention and related genes that are capable of inducing abiotic stress tolerance, and/or larger size.

After a dicot plant, monocot plant or plant cell has been transformed (and the latter regenerated into a plant) and shown to have greater size, or tolerate greater planting density, or have improved tolerance to abiotic stress, or improved water use efficiency, or to produce greater yield relative to a control plant, the transformed plant may be crossed with itself or a plant from the same line, a non-transformed or wild-type plant, or another transformed plant from a different transgenic line of plants.

The functions of specific polypeptides of the invention, including closely-related orthologs, have been analyzed and may be further characterized and incorporated into crop plants. Knocking down or knocking out of the expression of these sequences, or overexpression of these sequences, may be regulated using constitutive, inducible, or tissue specific regulatory elements. Genes that have been examined and have been shown to modify plant traits (including increasing yield and/or abiotic stress tolerance) encode polypeptides found in the Sequence Listing. In addition to these sequences, it is expected that newly discovered polynucleotide and polypeptide sequences closely related to polynucleotide and polypeptide sequences found in the Sequence Listing can also confer alteration of traits in a similar manner to the sequences found in the Sequence Listing, when transformed into any of a considerable variety of plants of different species, and including dicots and monocots. The polynucleotide and polypeptide sequences derived from monocots (e.g., the rice sequences) may be used to transform both monocot and dicot plants, and those derived from dicots (e.g., the Arabidopsis and soy genes) may be used to transform either group, although it is expected that some of these sequences will function best if the gene is transformed into a plant from the same group as that from which the sequence is derived.

As an example of a first step to determine water deprivation-related tolerance, seeds of these transgenic plants may be subjected to assays to measure sucrose sensing, severe desiccation tolerance, WUE, or drought tolerance. The methods for sucrose sensing, severe desiccation, WUE, or drought assays are described above. Sequences of the invention, that is, members of the HY5, STH2 and COP1 clades (e.g., SEQ ID NOs: 1-26, 48 and 50), may also be used to generate transgenic plants that are more tolerant to low nitrogen conditions or cold than control plants. Plants which are more tolerant than controls to water deprivation assays, low nitrogen conditions or cold are greener, more vigorous, or will have better survival rates than controls, or will recover better from these treatments than control plants.

All of these abiotic stress tolerances conferred by suppressing or knocking out expression of HY5 or STH2 or their closely related sequences, or increasing COP1 or its closely related sequences, may contribute to increased yield of commercially available plants. Thus, it is expected that altering expression of members of the HY5, STH2 and COP1 clades will improve yield in plants relative to control plants, including in leguminous species, even in the absence of overt abiotic stresses.

It is expected that the same methods may be applied to identify other useful and valuable sequences of the present polypeptide clades, and the sequences may be derived from a diverse range of species.

### Example XV

### Field Plot Designs, Harvesting and Yield Measurements of Soybean

A field plot of soybeans with any of various configurations and/or planting densities may be used to measure crop yield. For example, 30-inch-row trial plots consisting of multiple rows, for example, four to six rows, may be used for determining yield measurements. The rows may be approximately 20 feet long or less, or 20 meters in length or longer. The plots may be seeded at a measured rate of seeds per acre, for example, at a rate of about 100,000, 200,000, or 250,000 seeds/acre, or about 100,000-250,000 seeds per acre (the latter range is about 250,000 to 620,000 seeds/hectare).

Harvesting may be performed with a small plot combine or by hand harvesting. Harvest yield data are generally collected from inside rows of each plot of soy plants to measure yield, for example, the innermost inside two rows. Soybean yield may be reported in bushels (60 pounds) per acre. Grain moisture and test weight are determined; an electronic moisture monitor may be used to determine the moisture content, and yield is then adjusted for a moisture content of 13 percent (130 g/kg) moisture. Yield is typically expressed in bushels per acre or tonnes per hectare. Seed may be subsequently processed to yield component parts such as oil or carbohydrate, and this may also be expressed as the yield of that component per unit area.

For determining yield of maize, varieties are commonly planted at a rate of 15,000 to 40,000 seeds per acre (about 37,000 to 100,000 seeds per hectare), often in 30 inch rows. A common sampling area for each maize variety tested is with rows of 30 in. per row by 50 or 100 or more feet. At physiological maturity, maize grain yield may also be measured from each of number of defined area grids, for example, in each of 100 grids of, for example, 4.5 m² or larger. Yield measurements may be determined using a combine equipped with an electronic weigh bucket, or a combine harvester fitted with a grain-flow sensor. Generally, center rows of each test area (for example, center rows of a test plot or center rows of a grid) are used for yield measurements. Yield is typically expressed in bushels per acre or tonnes per hectare. Seed may be subsequently processed to yield component parts such as oil or carbohydrate, and this may also be expressed as the yield of that component per unit area.

### Example XVI

### Plant Expression Constructs for Downregulation of HY5 and HY5 Homologs

The technique of RNA interference (RNAi) may be applied to down-regulate target genes in plants. Typically, a plant expression construct containing, in 5′ to 3′ order, either a constitutive (e.g. CaMV 35S), environment-inducible (e.g. RD29A), or tissue-enhanced promoter (e.g. RBCS3) fused to an “inverted repeat” of a target DNA sequence and fused to a terminator sequence, is introduced into the plant via a standard transformation approach. Transcription of the sequence introduced via the expression construct within the plant cell leads to expression of an RNA species that folds back upon itself and which is then processed by the cellular machinery to yield small molecules that result in a reduction in transcript levels and/or translation of the endogenous gene products being targeted. P21103 is an example base vector that is used for the creation of RNAi constructs; the polylinker and PDK intron sequences in this vector are provided as SEQ ID NO: 118. The PDK intron in this vector is derived from pKANNIBAL (Wesley et al., 2001). RNAi constructs can be generated as follows: the target sequence is first amplified with primers containing restriction sites. A sense fragment is inserted in front of the Pdk intron using SalI/EcoRI to generate an intermediate vector, after which the same fragment is then subcloned into the intermediate vector behind the PDK intron in the antisense orientation using XbaI/EcoRI. Target sequences are typically selected to be 100 bp long or longer. For constructs designed against a clade rather than a single gene, the target sequences are usually chosen such that they have at least 85% identity to all clade members. Where it is not possible to identify a single 100 bp sequence with 85% identity to all clade members, hybrid fragments composed of two shorter sequences may be used. An example of an expressed sequence designed to target downregulation of HY5 and/or its homologs is provided as SEQ ID NO: 119.

A particular application of the present invention is to enhance yield by targeted down regulation of HY5 homologs in soybean by RNAi. Example nucleotide sequences suitable for targeting soybean HY5 homologs by an RNAi approach are provided in SEQ ID NOs: 116, the Gm_Hy5 RNAi target sequence, and SEQ ID NO: 117, the Gm_Hyh RNAi target sequence.”

### REFERENCES CITED

Aldemita and Hodges (1996) Planta 199: 612-617
Alia et al. (1998) Plant J. 16: 155-161
Alonso et al. (2003) Science 301: 653-657
Altschul (1990) J. Mol. Biol. 215: 403-410
Altschul (1993) J. Mol. Evol. 36: 290-300
Anderson and Young (1985) “Quantitative Filter Hybridisation”, In: Hames and Higgins, ed., Nucleic Acid Hybridisation, A Practical Approach. Oxford, IRL Press, 73-111
Ang et al. (1998) Mol. Cell. 1: 213-222
Ang and Deng (1994) Plant Cell 6: 613-628
Ausubel et al. (1997) Short Protocols in Molecular Biology, John Wiley & Sons, New York, N.Y., unit 7.7
Bairoch et al. (1997) Nucleic Acids Res. 25: 217-221
Baulcombe (1999) Curr. Opin. Plant Biol. 2: 109-113
Bechtold and Pelletier (1998) Methods Mol. Biol. 82: 259-266
Benhamed et al. (2006) Plant Cell 18, 2893-2903
Berger and Kimmel (1987), “Guide to Molecular Cloning Techniques”, in Methods in Enzymology, vol. 152, Academic Press, Inc., San Diego, Calif.
Bevan (1984) Nucleic Acids Res. 12: 8711-8721
Borden et al. (1995) EMBO J. 14: 5947-5956.
Cardoza and Steward (1992) Plant Cell Reports 21: 599-604
Casas et al. (1993) Proc. Natl. Acad. Sci. USA 90: 11212-11216
Chase et al. (1993) Ann. Missouri Bot. Gard. 80: 528-580
Chattopadhyay et al. (1998) Plant Cell 10: 673-683
Coruzzi et al. (2001) Plant Physiol. 125: 61-64
Christou et al. (1987) Proc. Natl. Acad. Sci. USA 84: 3962-3966
Christou (1991) Bio/Technol. 9: 957-962
Christou et al. (1992) Plant. J. 2: 275-281
D'Halluin et al. (1992) Plant Cell 4: 1495-1505
Daly et al. (2001) Plant Physiol. 127: 1328-1333
Datta et al. (2007) Plant Cell 19: 3242-3255
De Blaere et. al. (1987) “Vectors for Cloning in Plant Cells”, Meth. Enzymol., vol. 153:277-292
Deng et al. (1992) Cell 71: 791-801
Deshayes et al. (1985) EMBO J., 4: 2731-2737
Donn et al. (1990) in Abstracts of VIIth International Congress on Plant Cell and Tissue Culture IAPTC, A2-38: 53
Doolittle, ed. (1996) Methods in Enzymology, vol. 266: “Computer Methods for Macromolecular Sequence Analysis” Academic Press, Inc., San Diego, Calif., USA
Draper et al. (1982) Plant Cell Physiol. 23: 451-458
Eddy (1996) Curr. Opin. Str. Biol. 6: 361-365
Eisen (1998) Genome Res. 8: 163-167
Feng and Doolittle (1987) J. Mol. Evol. 25: 351-360
Fowler and Thomashow (2002) Plant Cell 14: 1675-1690
Franklin et al. (2005) Int. J. Dev. Biol. 49, 653-664
Fromm et al. (1990) Bio/Technol. 8: 833-839
Gilmour et al. (1998) Plant J. 16: 433-442
Gelvin et al. (1990) Plant Molecular Biology Manual, Kluwer Academic Publishers
Glantz (2001) Relative risk and risk score, in Primer of Biostatistics. 5^{th} ed., McGraw Hill/Appleton and Lange, publisher.
Glick and Thompson (1993) Methods in Plant Molecular Biology and Biotechnology. eds., CRC Press, Inc., Boca Raton
Goodrich et al. (1993) Cell 75: 519-530
Gordon-Kamm et al. (1990) Plant Cell 2: 603-618
Gruber et al., in Glick and Thompson (1993) Methods in Plant Molecular Biology and Biotechnology. eds., CRC Press, Inc., Boca Raton
Haake et al. (2002) Plant Physiol. 130: 639-648
Hain et al. (1985) Mol. Gen. Genet. 199: 161-168
Hardtke et al. (2000) EMBO J. 19, 4997-5006
Haymes et al. (1985) Nucleic Acid Hybridization: A Practical Approach, IRL Press, Washington, D.C.
Hein (1990) Methods Enzymol. 183: 626-645
Henikoff and Henikoff (1989) Proc. Natl. Acad. Sci. USA 89:10915
Henikoff and Henikoff (1991) Nucleic Acids Res. 19: 6565-6572
Herrera-Estrella et al. (1983) Nature 303: 209
Hiei et al. (1994) Plant J. 6:271-282
Hiei et al. (1997) Plant Mol. Biol. 35:205-218
Higgins and Sharp (1988) Gene 73: 237-244
Higgins et al. (1996) Methods Enzymol. 266: 383-402
Holm et al. (2001) EMBO J. 20:118-127
Holm et al. (2002) Genes & Dev. 16: 1247-1259
Hosmer and Lemeshow (1999) Applied Survival Analysis: regression Modeling of Time to Event Data. John Wiley & Sons, Inc. Publisher.
Hsieh et al. (1998) Proc. Natl. Acad. Sci. USA 95: 13965-13970
Ishida (1990) Nature Biotechnol. 14:745-750
Jakoby et al. (2002) Trends in Plant Sci. 7:106-111
Jang et al. (1997) Plant Cell 9: 5-19
Jiao et al. (2007) Nat. Rev. Gen. 8: 217-230
Kashima et al. (1985) Nature 313: 402-404
Kimmel (1987) Methods Enzymol. 152: 507-511
Klein et al. (1987) U.S. Pat. No. 4,945,050
Klee (1985) Bio/Technology 3: 637-642
Koornneef et al. (1980) Z. Pflanzen-physiol. 100, 147-160
Koornneef et al (1986) In Tomato Biotechnology: Alan R. Liss, Inc., 169-178
Ku et al. (2000) Proc. Natl. Acad. Sci. USA 97: 9121-9126
Lee et al. (2007) Plant Cell 19: 731-749
Leon-Kloosterziel et al. (1996) Plant Physiol. 110: 233-240
Lin et al. (1991) Nature 353: 569-571
Liu and Zhu (1997) Proc. Natl. Acad. Sci. USA 94: 14960-14964
McCallum et al. (2000) Nature Biotech. 18, 455-457
McNellis et al. (1994) Plant Cell 6: 487-500
McNellis et al. (1994b) Plant Cell 6: 1391-1400
Meyers (1995) Molecular Biology and Biotechnology, Wiley VCH, New York, N.Y., p 856-853
Miki et al. (1993) in Methods in Plant Molecular Biology and Biotechnology, p. 67-88, Glick and Thompson, eds., CRC Press, Inc., Boca Raton
Mount (2001), in Bioinformatics: Sequence and Genome Analysis, Cold Spring Harbor Laboratory Press, Cold Spring Harbor, N.Y., p. 543
Nienhuis et al. (1994) Am. J. Bot. 81, 943-947.
Osterlund et al. (2000) Nature 405: 462-466
Oyama et al. (1997) Genes Dev. 11, 2983-2995
Quail (2000) Semin. Cell Dev. Biol. 11, 457-466
Quail (2002a) Curr. Opin. Cell Biol. 14, 180-188
Quail (2002b) Nat. Rev. Mol. Cell. Biol. 3, 85-93
Ratcliffe et al. (2001) Plant Physiol. 126: 122-132
Reeves and Nissen (1995) Prog. Cell Cycle Res. 1: 339-349
Riechmann et al. (2000a) Science 290, 2105-2110
Riechmann, J. L., and Ratcliffe, O. J. (2000b) Curr. Opin. Plant Biol. 3, 423-434
Rieger et al. (1976) Glossary of Genetics and Cytogenetics: Classical and Molecular, 4th ed., Springer Verlag, Berlin
Sadowski et al. (1988) Nature 335: 563-564
Saleki et al. (1993) Plant Physiol. 101: 839-845
Sambrook et al. (1989) Molecular Cloning: A Laboratory Manual, 2nd Ed., Cold Spring Harbor Laboratory, Cold Spring Harbor, N.Y. Schroeder et al. (2002) Current Biol. 12, 1462-1472
Sanford et al. (1987) Part. Sci. Technol. 5:27-37
Sanford (1993) Methods Enzymol. 217: 483-509
Schroeder et al. (2002) Current Biol. 12: 1462-1472
Shin et al. (2007) Plant J. 49, 981-994
Shpaer (1997) Methods Mol. Biol. 70: 173-187
Smeekens (1998) Curr. Opin. Plant Biol. 1: 230-234
Smith et al. (1992) Protein Engineering 5: 35-51
Soltis et al. (1997) Ann. Missouri Bot. Gard. 84: 1-49
Somers et al. (1991) Plant Cell 3, 1263-1274
Sonnhammer et al. (1997) Proteins 28: 405-420
Spencer et al. (1994) Plant Mol. Biol. 24: 51-61
Stitt (1999) Curr. Opin. Plant. Biol. 2: 178-186
Tepperman et al. (2001) Proc Natl Acad Sci U S A., 98, 9437-9442
Tepperman et al. (2004) Plant J., 38, 725-739
Thompson et al. (1994) Nucleic Acids Res. 22: 4673-4680
Torok and Etkin et al. (2001) Differentiation 67: 63-71.
Tudge (2000) in The Variety of Life, Oxford University Press, New York, N.Y. pp. 547-606
Vasil et al. (1992) Bio/Technol. 10:667-674
Vasil et al. (1993) Bio/Technol. 11:1553-1558
Vasil (1994) Plant Mol. Biol. 25: 925-937
von Arnim and Deng (1994) Trends Cell Biol. 15, 618-625
Wahl and Berger (1987) Methods Enzymol. 152: 399-407
Wan and Lemeaux (1994) Plant Physiol. 104: 37-48
Weeks et al. (1993) Plant Physiol. 102:1077-1084
Weissbach and Weissbach (1989) Methods for Plant Molecular Biology, Academic Press
Wesley et al. (2001). Plant J 27: 581-590
Wu (ed.) Meth. Enzymol. (1993) vol. 217, Academic Press
Wu et al. (1996) Plant Cell 8: 617-627
Xin and Browse (1998) Proc. Natl. Acad. Sci. USA 95: 7799-7804
Yi and Deng (2005) Trends Cell Biol. 15, 618-625.
Zhang et al. (1991) Bio/Technology 9: 996-997
Zhu et al. (1998) Plant Cell 10: 1181-1191

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A nucleic acid construct comprising a recombinant nucleic acid sequence, wherein introduction of the nucleic acid construct into a plant results in a reduction or abolition of expression of a HY5 or STH2 clade member polypeptide as compared to a control plant;
wherein the HY5 clade member polypeptide:
is encoded by a polynucleotide that hybridizes to SEQ ID NO: 2 under stringent conditions; or
comprises a V-P-E/D-φ-G domain having an amino acid identity to amino acids 35-47 of SEQ ID NO: 2, and a bZIP domain having an amino acid identity to amino acids 78-157 of SEQ ID NO: 2; or
or has an amino acid identity to SEQ ID NO: 2;
and wherein the STH2 clade member polypeptide:
is encoded by a polynucleotide that hybridizes to SEQ ID NO: 24 under stringent conditions; or
comprises two B-box domains and the first B-box domain having an amino acid identity to amino acids 2-33 of SEQ ID NO: 24 and the second B-box domain having an amino acid identity to amino acids 60-102 of SEQ ID NO: 24; or
has an amino acid identity to SEQ ID NO: 24; and
the amino acid identity is selected from the group consisting of at least: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%; and
said plant exhibits increased yield, increased germination, increased seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, as compared to the control plant.

2. The nucleic acid construct of claim 1, wherein the reduction or abolition of HY5 or STH2 clade member gene expression is achieved by co-suppression, with antisense constructs, with sense constructs, by RNAi, small interfering RNA, targeted gene silencing, molecular breeding, virus induced gene silencing (VIGS), overexpression of suppressors of one or more HY5 or STH2 clade member genes, by the overexpression of microRNAs that target one or more HY5 or STH2 clade member genes, or by genomic disruptions, including transposons, tilling, homologous recombination, or T-DNA insertion.

3. The nucleic acid construct of claim 1, wherein the nucleic acid construct encodes a polypeptide comprising any of SEQ ID NO: 2, 4, 6, 8, 10, 12, 24, 26, 48, or 50.

4. The nucleic acid construct of claim 1, wherein the nucleic acid construct is comprised within a recombinant host plant cell.

5. The nucleic acid construct of claim 1, wherein the nucleic acid construct is comprised within a transgenic seed, and a progeny plant grown from the transgenic seed exhibits greater yield, increased germination, seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, as compared to a control plant.

6. A nucleic acid construct comprising a recombinant nucleic acid sequence, wherein introduction of the nucleic acid construct into a plant results in greater expression or activity of a COP1 clade member polypeptide in the plant than in a control plant;
wherein the COP1 clade member polypeptide:
is encoded by a polynucleotide that hybridizes to SEQ ID NO: 14 under stringent conditions; or
comprises a RING domain having an amino acid identity to amino acids 51-93 of SEQ ID NO: 14, and a WD40 domain having an amino acid identity to amino acids 374-670 of SEQ ID NO: 14; or
has an amino acid identity to SEQ ID NO: 2; and
the amino acid identity is selected from the group consisting of at least: 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%; and
wherein said plant exhibits increased yield, increased germination, increased seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, as compared to the control plant.

7. The nucleic acid construct of claim 6, wherein the nucleic acid construct encodes a polypeptide comprising any of SEQ ID NO: 14, 16, 18, 20, or 22.

8. The nucleic acid construct of claim 6, wherein the nucleic acid construct is comprised within a recombinant host plant cell.

9. The nucleic acid construct of claim 6, wherein the nucleic acid construct is comprised within a transgenic seed, and a progeny plant grown from the transgenic seed exhibits greater yield, increased germination, increased seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, as compared to a control plant.

10. A method for altering a trait in a plant as compared to a control plant, wherein the altered trait is selected from the group consisting of greater yield, increased germination, increased seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, the methods steps including:
transforming a target plant with a nucleic acid construct that comprises:
(a) a recombinant nucleic acid sequence, wherein introduction of the nucleic acid construct into a plant results in a reduction or abolition of expression of a HY5 or STH2 clade member polypeptide as compared to a control plant;
wherein the HY5 clade member polypeptide:
is encoded by a polynucleotide that hybridizes to SEQ ID NO: 2 under stringent conditions; or
comprises a V-P-E/D-φ-G domain having an amino acid identity to amino acids 35-47 of SEQ ID NO: 2, and a bZIP domain having an amino acid identity to amino acids 78-157 of SEQ ID NO: 2; or
has an amino acid identity to SEQ ID NO: 2;
and wherein the STH2 clade member polypeptide:
is encoded by a polynucleotide that hybridizes to SEQ ID NO: 24 under stringent conditions; or
comprises two B-box domains and the first B-box domain has an amino acid identity to amino acids 2-33 of SEQ ID NO: 24 and the second B-box domain has an amino acid identity to amino acids 60-102 of SEQ ID NO: 24; or
has an amino acid identity to SEQ ID NO: 24; or
(b) a recombinant nucleic acid sequence, wherein introduction of the nucleic acid construct into a plant results in greater expression or activity of a COP1 clade member polypeptide in the plant than in a control plant;
wherein the COP1 clade member polypeptide:
is encoded by a polynucleotide that hybridizes to SEQ ID NO: 14 under stringent conditions; or
comprises a RING domain having an amino acid identity to amino acids 51-93 of SEQ ID NO: 14, and a WD40 domain having an amino acid identity to amino acids 374-670 of SEQ ID NO: 14; or
has an amino acid identity to SEQ ID NO: 2; and
the amino acid identity is selected from the group consisting of at least: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%; and
said plant has reduced or abolished expression of a HY5 or STH2 clade member gene, and said reduced or abolished expression of the HY5 or STH2 clade member gene alters the trait in the plant as compared to a control plant, or greater expression of a COP1 clade member sequence, and said greater expression of the COP1 clade member alters the trait in the plant as compared to a control plant.

11. The method of claim 10, wherein the method steps further comprise selfing or crossing the transgenic knockdown or knockout plant with itself or another plant, respectively, to produce a transgenic seed.

12. A plant exhibiting an altered trait as compared to the control plant, wherein the altered trait is selected from the group consisting of greater yield, greater height of the mature plant, increased secondary rooting, greater cold tolerance, greater tolerance to water deprivation, reduced stomatal conductance, altered C/N sensing, increased low nitrogen tolerance, reduced percentage of hard seed, greater average stem diameter, increased stand count, improved late season growth and vigor, increased number of pod-bearing main-stem nodes, greater late season canopy coverage, and increased tolerance to hyperosmotic stress, or combinations thereof;
wherein the plant is derived from a plant or plant cell that has previously been specifically selected based on its having greater expression or activity of a COP1 clade member polypeptide, or reduced or abolished expression or activity of a HY5 clade member polypeptide or an STH2 clade member polypeptide, as compared to the control plant;
wherein the COP1 clade member polypeptide:
is encoded by a polynucleotide that hybridizes to SEQ ID NO: 14 under stringent conditions; or
comprises a RING domain having an amino acid identity to amino acids 51-93 of SEQ ID NO: 14, and a WD40 domain having an amino acid identity to amino acids 374-670 of SEQ ID NO: 14; or
has an amino acid identity to SEQ ID NO: 2;
wherein the HY5 clade member polypeptide:
is encoded by a polynucleotide that hybridizes to SEQ ID NO: 2 under stringent conditions; or
comprises a V-P-E/D-φ-G domain having an amino acid identity to amino acids 35-47 of SEQ ID NO: 2, and a bZIP domain having an amino acid identity to amino acids 78-157 of SEQ ID NO: 2; or
has an amino acid identity to SEQ ID NO: 2;
and wherein the STH2 clade member polypeptide:
is encoded by a polynucleotide that hybridizes to SEQ ID NO: 24 under stringent conditions; or
comprises two B-box domains and the first B-box domain having an amino acid identity to amino acids 2-33 of SEQ ID NO: 24 and the second B-box domain having an amino acid identity to amino acids 60-102 of SEQ ID NO: 24; or
has an amino acid identity to SEQ ID NO: 24, and
the amino acid identity is selected from the group consisting of at least: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%.

13. The plant of claim 12, wherein the reduced or abolished expression or activity of a HY5 clade member polypeptide or an STH2 clade member polypeptide is achieved by co-suppression, by chemical mutagenesis, by fast neutron deletion, with antisense constructs, with sense constructs, by RNAi, small interfering RNA, targeted gene silencing, molecular breeding, tilling, virus induced gene silencing (VIGS), overexpression of suppressors of HY5, or STH2 clade member gene, by the overexpression of microRNAs that target HY5, or STH2 clade member gene, or by genomic disruptions, including transposons, tilling, homologous recombination, DNA-repair related processes, or T-DNA insertion.

14. The plant of claim 12, wherein the plant has a deletion within a portion of its genome encoding the entirety of, or a portion of, a HY5 or STH2 clade member polypeptide.

15. A genetically modified or transgenic knockout plant, the genome of which comprises a disruption within an endogenous HY5 or STH2 clade member gene or within the regulatory regions of said gene, wherein said disruption prevents normal function of an endogenous HY5 or STH2 clade member polypeptide and results in said knockout plant exhibiting increased yield, increased germination, increased seedling vigor, greater height of the mature plant, increased secondary rooting, increased plant stand count, thicker stem, lodging resistance, increased number of nodes, greater cold tolerance, greater tolerance to water deprivation, reduced stomatal conductance, altered C/N sensing, increased low nitrogen tolerance, increased tolerance to hyperosmotic stress, delayed senescence, alteration in the levels of photosynthetically active pigments, improved seed quality, reduced percentage of hard seed, greater average stem diameter, increased stand count, improved late season growth or vigor, increased number of pod-bearing main-stem nodes, greater late season canopy coverage, or combinations thereof, as compared to a control plant.
